(19) United States
(12) Patent Application Publication
(10) Pub. No.: US 2014/0271560 A1
(43) Pub. Date: Sep. 18, 2014

(54) Methods for Using Cryptococcus Flavescens Strains for Biological Control of Fusarium Head Blight

(71) Applicants: Ohio State Innovation Foundation, Columbus, OH (US); The United States of America, as Represented by the Secreary of Agirculture, Washington, DC (US)

(72) Inventors: Brian B. McSpadden Gardener, Wooster, OH (US); Pierce Anderson Paul, Wooster, OH (US); Michael J. Boehm, Worthington, OH (US); Xiaoqing Rong, Wooster, OH (US); David Schisler, Morton, IL (US)

(73) Assignees: Ohio State Innovation Foundation, Columbus, OH (US); The United States of America, as Represented by the Secretary of Agriculture, Washington, DC (US)

(21) Appl. No.: 14/211,053

(22) Filed: Mar. 14, 2014

Related U.S. Application Data
(60) Provisional application No. 61/787,458, filed on Mar. 15, 2013.

(51) Int. Cl. C12Q 1/68

(52) U.S. Cl. CPC C12Q 1/689
USPC 424/93.4; 435/6.11

(57) ABSTRACT

Disclosed are methods of identifying subspecies of Cryptococcus flavescens and methods of treating or suppressing Fusarium head blight with the different Cryptococcus flavescens species. In particular, two genotypes, Genotypes A and B, were identified using the disclosed real time PCR technique. The following Cryptococcus flavescens strains were identified as being either Genotype A or B and as being able to suppress Fusarium head blight: NRRL Y-7373, YB-601, YB-602, Y-7377, Y-7372, Y-7375, Y-7374, Y-7376, YB-328, Y-7379, and YB-744.

### CROSS-REFERENCE TO RELATED APPLICATIONS

This application claims priority to U.S. Provisional Application No. 61/787,458, filed on Mar. 15, 2013, the entire disclosure of which is expressly incorporated herein by reference for all purposes.

### SEQUENCE LISTING

The instant application contains a Sequence Listing which has been submitted via EFS-web and is hereby incorporated by reference in its entirety. The ASCII copy, created on Mar. 12, 2014, is named 1-55890_SL.txt, and is 53,393 bytes in size.

### BACKGROUND OF THE INVENTION

Head scab, also known as Fusarium head blight (FHB), is a devastating disease of wheat and barley that is primarily caused by the fungus Gibberella zeae (anamorph=Fusarium graminearum). This disease can reach epidemic levels and causes extensive damage to wheat and barley in humid and semi-humid wheat growing areas of the world. The disease has caused large scale devastation in the United States, Canada and China. Other countries of the world that produce large amounts of wheat in humid and semi-humid regions and would be susceptible to major outbreaks of FHB include India, Russia, France, Germany and the United Kingdom. FHB was responsible for almost 500 million bushels of wheat lost in the United States from 1991 until 2013. Economic loss has been estimated at 1.3 to 2.6 billion during this time period. The importance of FHB was recognized by the 105th U.S. Congress when it adopted the “Wheat and Barley Protection Act” that authorized expenditure of 26 million dollars for the study of FHB.

A number of different strategies have been employed to manage and control plant pathogens such as FHB. Chemical pesticides are often relied on to help control these pathogens. However, the widespread use of chemicals in agriculture has been a subject of growing public concern and scrutiny due to the potentially harmful effects on the environment and human health. Other problems linked to pesticide use, including the emergence of pesticide-resistant pathogens, have led to a gradual elimination of some available pesticides. In addition, there presently exist strict regulations on chemical pesticide use, and political pressure aiming to remove the most hazardous chemicals from the market. As a result, some chemical companies have become increasingly reluctant to develop and test new chemicals due to the concerns relating to registration process and cost. Alternatively, the practice of conventional tillage has been used against FHB. None of these approaches have led to substantial and consistent control of FHB. The residues and high cost of chemical fungicides and the desires for reduced or conservation tillage to prevent soil erosion and runoff discourage the use of these strategies.

There is a clear need for the development of non-chemical alternative methods to control plant pathogenic diseases, such as FHB. Biological control of plant pathogens has been increasingly considered a viable alternative to manage plant diseases. Particularly, the use of biologically active agents in the control of plant pests and pathogenic diseases has become especially important for certified organic growers who do not use synthetic chemicals for pest management.

Accordingly, disclosed herein are methods of using Cryptococcus flavescens strains to suppress FHB on cereal plants.

### SUMMARY OF THE INVENTION

Disclosed are methods of identifying subspecies of Cryptococcus flavescens and methods of treating or suppressing Fusarium head blight with the different Cryptococcus flavescens strains. In particular, two genotypes, Genotypes A and B, were identified using the disclosed real time PCR technique. The following Cryptococcus flavescens strains were identified as being either Genotype A or B and as being able to suppress Fusarium head blight: Y-7373, YB-601, YB-602, Y-7377, Y-7372, Y-7375, Y-7374, Y-7376, YB-328, Y-7379, and YB-744.

In a particular embodiment described herein is a method for suppressing Fusarium head blight in a cereal plant comprising: a) identifying a genotype for a Cryptococcus flavescens strain by qPCR and/or by sequence identity; and b) applying to a seed head of said plant an effective amount of one or more microbial antagonists, wherein the one or more microbial antagonist is a Genotype A Cryptococcus flavescens strain or Genotype B Cryptococcus flavescens strain, as identified in step a), wherein the one or more microbial antagonists is not Cryptococcus flavescens 3C, which has been deposited under NRRL accession no. Y-50378, or Cryptococcus flavescens 4C, which has been deposited under NRRL accession no. Y-50379.

In another embodiment described herein, the genotype for a Cryptococcus flavescens strain is determined by quantitative PCR (qPCR), comprising the steps: a) performing qPCR on extracted genomic DNA utilizing one or more primer pairs sharing at least 90% sequence identity with the primer pairs of Table 1 (SEQ ID NOs: 45-72), wherein sequence identity is determined for each individual forward or reverse primer; and b) identifying a Cryptococcus flavescens strain as Genotype A or Genotype B, wherein: (1) the Cryptococcus flavescens strain is identified as Genotype A when qPCR results in: a threshold cycle value of 17-21 when using the btub.1, btub.2, or EF1.2 primer sets; a threshold cycle value of 18-20 when using the h22.1, h30.2, or h31.1 primer sets; a threshold cycle value of 17-20 when using the h31.2 primer set; or a threshold cycle value of 12-15 when using the 12.1 primer set; and (2) the Cryptococcus flavescens strain is identified as Genotype B when qPCR results in a threshold cycle value of 31-33 when using the btub.1 primer set; a threshold cycle value of 32-35 when using the btub.2 primer set; a threshold cycle value of 24-26 when using the EF1.2 primer set; a threshold cycle value of 22-24 when using the h22.1 or h31.1 primer sets; a threshold cycle value of 22-23 when using the h30.2 primer set; a threshold cycle value of 34-35 when using the h31.2 primer set; or a threshold cycle value of 15-18 when using the I2.1 primer set.

In another embodiment described herein, the genotype for a Cryptococcus flavescens strain is determined by sequence identity, comprising the steps: a) sequencing extracted genomic DNA of a target Cryptococcus flavescens; b) comparing the sequence determined in step a) to known homologous sequences of other Cryptococcus flavescens strains; and c) identifying a Cryptococcus flavescens strain as Genotype A or Genotype B, wherein: (1) the Cryptococcus flavescens strain is identified as Genotype A when the sequence of the extracted genomic DNA of the target Cryptococcus flavescens shares: 100% sequence identity at the β-tubulin gene to SEQ ID NO:1; between 95% and 100% sequence identity at the chitin synthase 1 gene to SEQ ID NOs: 2, 3, 4, 5, or 6; between 99% and 100% sequence identity at the EF1 gene to SEQ ID NOs: 7, 8, 9, 10, or 11; between 99% and 100% sequence identity at the hsp70 gene to SEQ ID NOs: 12, 13, 14, 15, or 16; or between 99% and 100% sequence identity at the cs22 target locus to SEQ ID. NOs: 85, 86, 87, 88, or 89; and (2) the Cryptococcus flavescens strain is identified as Genotype B when the sequence of the extracted genomic DNA of the target Cryptococcus flavescens shares: between 99% and 100% sequence identity at the β-tubulin gene to SEQ ID NOs: 17, 18, 19, 20, 21, 22 or 23; between 97% and 100% sequence identity at the chitin synthase 1 gene to SEQ ID NOs: 24, 25, 26, 27, 28, 29 or 30; between 99% and 100% sequence identity at the EF1 gene to SEQ ID NOs: 31, 32, 33, 34, 35, 36, or 37; between 99% and 100% sequence identity at the hsp70 gene to SEQ ID NOs: 38, 39, 40, 41, 42, 43 or 44; or 100% sequence identity at the cs22 target locus to SEQ ID NO: 90.

In another embodiment described herein, the extracted genomic DNA is collected from one or more cereal grass plants prior to application of one or more microbial antagonists to the one or more cereal grass plants.

In another embodiment described herein, the extracted genomic DNA is collected from one or more cereal grass plants following application of one or more microbial antagonists to the one or more cereal grass plants.

In another embodiment described herein, the extracted genomic DNA is collected from an in vitro Cryptococcus flavescens culture.

In another embodiment described herein, at least one of the one or more microbial antagonists is a Genotype A Cryptococcus flavescens strain.

In another embodiment described herein, at least one of the one or more microbial antagonists is a Genotype B Cryptococcus flavescens strain.

In another embodiment described herein, at least one of the one or more microbial antagonists is selected from the group of C. flavescens strains consisting of: Y-7373; YB-601; YB-602; Y-7377; Y-7372; Y-7375; Y-7374; Y-7376; YB-328; Y-7379; and YB-744.

In another embodiment described herein, at least one of the one or more microbial antagonists is tolerant to prothioconazole.

In another embodiment described herein, at least one of the one or more microbial antagonist is C. flavescens Y-7373.

In another embodiment described herein, at least one of the one or more microbial antagonist is C. flavescens YB-601.

In another embodiment described herein, at least one of the one or more microbial antagonist is C. flavescens YB-602.

In another embodiment described herein, at least one of the one or more microbial antagonist is C. flavescens Y-7377.

In another embodiment described herein, at least one of the one or more microbial antagonist is C. flavescens Y-7372.

In another embodiment described herein, at least one of the one or more microbial antagonist is C. flavescens Y-7375.

In another embodiment described herein, at least one of the one or more microbial antagonist is C. flavescens Y-7374.

In another embodiment described herein, at least one of the one or more microbial antagonist is C. flavescens Y-7376.

In another embodiment described herein, at least one of the one or more microbial antagonist is C. flavescens YB-328.

In another embodiment described herein, at least one of the one or more microbial antagonist is C. flavescens Y-7379.

In another embodiment described herein, at least one of the one or more microbial antagonist is C. flavescens YB-744.

In another embodiment described herein, in addition to the one or more microbial antagonists being applied to a seed head of a cereal plant, Cryptococcus flavescens 3C, which has been deposited under NRRL accession no. Y-50378, and/or Cryptococcus flavescens 4C, which has been deposited under NRRL accession no. Y-50379, is applied to the cereal plant.

In another embodiment described herein, the one or more microbial antagonists are applied to the seed head prior to a hard dough stage of development.

In another embodiment described herein, the one or more microbial antagonists are applied to the seed head during flowering.

In another embodiment described herein, the one or more microbial antagonists are applied to the seed head prior to flowering.

In another embodiment described herein, the cereal plant is wheat or barley.

In another embodiment described herein, the cereal plant is wheat.

In another embodiment described herein, the effective amount of at least one microbial antagonist is an amount sufficient to reduce the level of Fusarium head blight relative to that in a corresponding untreated control.

In another embodiment described herein, application of an effective amount of one or more microbial antagonists to the seed head of a cereal plant comprises spraying the one or more microbial antagonists onto the cereal plant.

In another embodiment described herein, the method of spraying is selected from the group consisting of: spraying through a sprinkler irrigation system; aerial spray application; ground-based spray application.

In another embodiment described herein, the effective amount of the one or more microbial antagonists is between about 10⁴-10⁹ CFU/ml applied at a rate of about 10⁵-10⁶ CFU/cm².

In another embodiment described herein, the effective amount of the one or more microbial antagonists is about 1.5×10⁹ CFU/ml applied at a rate of about 2×10⁶ to 6×10⁶ CFU/cm².

In another embodiment described herein, the effective amount of the one or more microbial antagonists is about 2.3×10⁸ CFU/ml applied at a rate of about 2×10⁵ CFU/cm².

In another embodiment described herein, the effective amount of the one or more microbial antagonists is about 3×10⁸ CFU/ml at a rate of about 10⁶ CFU/cm².

In another embodiment described herein, application of an effective amount of the one or more microbial antagonists to the seed head of a cereal plant occurs at temperatures between about 5 to 35° C.

In another embodiment described herein, application of an effective amount of the one or more microbial antagonists to the seed head of a cereal plant occurs at temperatures between about 15 to 30° C.

In another embodiment described herein, the one or more microbial antagonists are substantially biologically pure.

In another embodiment described herein, two or more microbial antagonists are applied to the seed head of the cereal plant.

In another embodiment described herein, the two or more microbial antagonists are applied simultaneously.

In another embodiment described herein, the two or more microbial antagonists are applied separately.

In another embodiment described herein, at least two microbial antagonists are Genotype A Cryptococcus flavescens strains.

In another embodiment described herein, at least two microbial antagonists are Genotype B Cryptococcus flavescens strains.

In another embodiment described herein, a first microbial antagonist is a Genotype A Cryptococcus flavescens strain and a second microbial antagonist is a Genotype B Cryptococcus flavescens strain.

In another embodiment described herein, at least one microbial antagonist is selected from a first group consisting of: Genotype A Cryptococcus flavescens strains; and Genotype B Cryptococcus flavescens strains, and at least one microbial antagonist is selected from a second group consisting of: Cryptococcus flavescens 3C, which has been deposited under NRRL accession no. Y-50378; and Cryptococcus flavescens 4C, which has been deposited under NRRL accession no. Y-50379.

In another embodiment described herein, the method for suppressing Fusarium head blight in a cereal plant further comprises applying one or more fungicides to the cereal plant.

In another embodiment described herein, the fungicide is prothioconazole.

In another embodiment described herein, the one or more fungicides are applied to the cereal plant at a time selected from the group consisting of: prior to application of the one or more microbial antagonists; simultaneously with the application of the one or more microbial antagonists; and subsequent to the application of the one or more microbial antagonists.

In a particular embodiment described herein is a kit comprising one or more microbial antagonists disclosed herein.

In a particular embodiment described herein is a kit comprising one or more primer pairs sharing at least 90% sequence identity with the primer pairs of Table 1 (SEQ ID NOs: 45-72), wherein sequence identity is determined for each individual forward or reverse primer.

In a particular embodiment described herein is a method for identifying the genotype of a Cryptococcus flavescens strain comprising: a) performing qPCR on extracted genomic DNA utilizing one or more primer pairs sharing at least 90% sequence identity with the primer pairs of Table 1 (SEQ ID NOs: 45-72), wherein sequence identity is determined for each individual forward or reverse primer; and b) identifying a Cryptococcus flavescens strain as Genotype A or Genotype B, wherein: (1) the Cryptococcus flavescens strain is identified as Genotype A when qPCR results in: a threshold cycle value of 17-21 when using the btub.1, btub.2, or EF1.2 primer sets; a threshold cycle value of 18-20 when using the h22.1, h30.2, or h31.1 primer sets; a threshold cycle value of 17-20 when using the h31.2 primer set; or a threshold cycle value of 12-15 when using the I2.1 primer set; and (2) the Cryptococcus flavescens strain is identified as Genotype B when qPCR results in a threshold cycle value of 31-33 when using the btub.1 primer set; a threshold cycle value of 32-35 when using the btub.2 primer set; a threshold cycle value of 24-26 when using the EF1.2 primer set; a threshold cycle value of 22-24 when using the h22.1 or h31.1 primer sets; a threshold cycle value of 22-23 when using the h30.2 primer set; a threshold cycle value of 34-35 when using the h31.2 primer set; or a threshold cycle value of 15-18 when using the I2.1 primer set.

In a particular embodiment described herein is a method for identifying the genotype of a Cryptococcus flavescens strain comprising: a) sequencing extracted genomic DNA of a target Cryptococcus flavescens; b) comparing the sequence determined in step a) to known homologous sequences of other Cryptococcus flavescens strains; and c) identifying a Cryptococcus flavescens strain as Genotype A or Genotype B, wherein: (1) the Cryptococcus flavescens strain is identified as Genotype A when the sequence of the extracted genomic DNA of the target Cryptococcus flavescens shares: 100% sequence identity at the β-tubulin gene to SEQ ID NO:1; between 95% and 100% sequence identity at the chitin synthase 1 gene to SEQ ID NOs: 2, 3, 4, 5, or 6; between 99% and 100% sequence identity at the EF1 gene to SEQ ID NOs: 7, 8, 9, 10, or 11; between 99% and 100% sequence identity at the hsp70 gene to SEQ ID NOs: 12, 13, 14, 15, or 16; or between 99% and 100% sequence identity at the cs22 target locus to SEQ ID. NOs: 85, 86, 87, 88, or 89; and (2) the Cryptococcus flavescens strain is identified as Genotype B when the sequence of the extracted genomic DNA of the target Cryptococcus flavescens shares: between 99% and 100% sequence identity at the β-tubulin gene to SEQ ID NOs: 17, 18, 19, 20, 21, 22 or 23; between 97% and 100% sequence identity at the chitin synthase 1 gene to SEQ ID NOs: 24, 25, 26, 27, 28, 29 or 30; between 99% and 100% sequence identity at the EF1 gene to SEQ ID NOs: 31, 32, 33, 34, 35, 36, or 37; between 99% and 100% sequence identity at the hsp70 gene to SEQ ID NOs: 38, 39, 40, 41, 42, 43 or 44; or 100% sequence identity at the cs22 target locus to SEQ ID NO: 90.

### BRIEF DESCRIPTION OF THE DRAWINGS

FIGS. 1A-1F: FIGS. 1A-1F show the genetic similarity among C. flavescens strains. The DNA sequences of Internal Transcribed Spacer (A) and a concatenation of β-tubulin, Chitin Synthase 1, Elongation Factor 1 and Heat Shock Protein 70 kDa (B) are shown. The homologous regions in C. neoformans JEC21 (GenBank Accession Numbers AE107341 to AE107356) were used as outgroup. The phylogenetic relationships among taxa were inferred by the Neighbor Joining method from distances computed using the Tamura-Nei model. Bootstrap values of >60% (1,000 replicates) are shown next to the branches. Scale bar shows number of base substitutions per site. C. flavescens strains split into two groups, shown individually by the DNA sequences of β-tubulin (C), Chitin Synthase 1 (D), Elongation Factor 1 (E) and Heat Shock Protein 70 kDa (F). The phylogenetic relationships among taxa were inferred by the Maximum Likelihood method with General Time Reversible model. Bootstrap values of >60% (1,000 replicates) are shown next to the branches. Scale bar shows number of base substitutions per site. Different bracket line patterns were used to differentiate 3C-type (homogenous dash) and non-3C type strains (alternative dash).

FIG. 2: FIG. 2 shows the sensitivity and specificity of the C. flavescens-specific qPCR assay targeting the putative hsp70 gene. Tested samples included 10-fold serial dilutions of 3C genomic DNA, 2.5 ng genomic DNA per reaction of other C. flavescens strains, template-free PCR water, and two field samples (*). The templates of the field samples are the 1:20 diluted DNA extracts from a 3C-inoculated wheat head (“w/ 3C”) 44 days post inoculation and a noninoculated head (“w/o 3C”) on the day of inoculation, respectively. The target amplicon is 150 bp in size, indicated by the arrow. Threshold Cycle was marked as “N/A” (Not Applicable) when fluorescence signal was below the threshold at the end of amplification. Melting temperature was shown as “-” when no peak of 87.0 or 87.5° C. (melting temperature of the standard series) was detected.

FIGS. 3A-3J: FIGS. 3A-3J are qPCR amplification curves. Contigs harboring sequences similar to internal transcribed spacer (ITS) regions, heat shock protein (hsp70), beta tubulin (btub), elongation factor 1 (EF1), and chitin synthase 5 were identified, and chosen as targets for the development of strain specific primers. FIG. 3A: btub.1; FIG. 3B: btub.2; FIG. 3C: EF1.1; FIG. 3D: EF1.2; FIG. 3E: h22.1; FIG. 3F: h30.2; FIG. 3G: h31.1; FIG. 3H: h31.2; FIG. 3I-5I; and FIG. 3J: I2.1.

FIG. 4: FIG. 4 shows the biocontrol efficacy of two distinct genotypes of Cryptococcus flavescens. Genotype A, which is the more effective genotype, includes the previously patented strain OH 182.9 (3C), as well as USDA isolates Y-7373, YB-601 and YB-602. Genotype B includes USDA isolates Y-7372, YB-328, and YB-744. Average disease severity values combined across five independent experiments are shown. A total of 227, 158, and 69 heads were assayed for genotypes A, genotypes B, and the negative control (nc), respectively. Error bars represent standard error of the means.

FIG. 5: FIG. 5 is a field map and major sampling scheme for the detection of C. flavescens strain 3C in Field One. Wheat (cultivar Hopewell) was planted throughout the field of 42.7 m by 61.0 m (the biggest rectangle), excluding the area of fallow walkways (indicated by double solid lines), which were 0.6-m wide between each 6.1-m stripes of wheat-grown area. Three areas of 6.1 m by 6.1 m were sprayed with 3C during anthesis of the wheat, indicated by the shaded areas labeled as “i_{—}1”, “i_{—}2” and “i_{—}3” in the map. In the summer of 2011, sampling was done within a rectangle of 30.5 m by 42.7 m (consisting of 5×7 square grids of solid lines) by clipping off one wheat head from each of the locations indicated approximately by the Roman numerals (inside of wheat-grown areas) and short thick lines (along the borders of wheat-grown areas neighboring walkways) in the map. Each unique Roman numeral indicates one group of sampling locations of the similar distances to the nearest points on inoculated areas. The mean distances of each group were: I—0 m, II—1.60 m, III—4.64 m, IV—7.38 m, V—12.37 m, VI—14.72 m and VII—18.12 m. Samples were collected from the locations of Group I to V on 0, 1, 10, 26 and 44 day(s) post inoculation. Group V, VI and VII were sampled only at 44 days post inoculation. The border locations (indicated by short thick lines) were only sampled at 10 days post inoculation.

FIG. 6: FIG. 6 is a graph showing the population dynamics of 3C on inoculated wheat heads. 3C inoculum suspension was sprayed on wheat at a rate of 2×10⁶ to 6×10⁶ cells per square centimeter. The wheat head samples (n=5 to 9) collected from the three inoculated areas (see FIG. 5 for details on sampling locations) at five time points post inoculation were compared. The median populations of the groups that do not share the same letter (a, b) were significantly different based on Tukey's test (P≦0.05). “DL” on the Y-axis represents the theoretical detection limit for this experiment. The number of sample that was negative for 3C detection at a given time point was indicated in a square box at the bottom of the plot.

FIGS. 7A-7B: Abundance of 3C-like C. flavescens in a wheat field over time and distance relative to the point of inoculation. In FIG. 7A, the mean percentage of samples above a threshold population level along a linear scale of distance (in meters) from inoculation points are shown. In FIG. 7B, the percentage of samples scoring positive for all colonization levels (colored bars) at all measured distances (see FIG. S1 for distance grouping shown in Roman numerals). Note, samples were taken from distances I to IV at 0 to 26 day(s) post inoculation (DPI) while additional samplings and distances V to VII are provided solely at 44 DPI. The sample size from each of the location groups at each time point were: for distance group I—n=5 to 9 (depending on date of sampling), II—n=18 to 20, III—n=8 to 10, IV—n=4 to 6, V—n=16, VI—n=8, and VII—n=4. The theoretical detection limit of this experiment was 2.7×10³ target gene copies per gram wheat head.

FIG. 8: FIG. 8 shows the overwintering persistence of 3C in the inoculated areas of the field. Postharvest residues of the three inoculated areas were sampled in late January and late March of 2012. Composite samples (C) of soil, living weeds and brownish rotting plant materials and samples of non-rotting wheat stalks (W) were collected within a circle of about 1-m radius around the center of each inoculated area. “DL” on the Y-axis represents the theoretical detection limit for this experiment. The numbers of samples that were negative for 3C detection for each sample type and time point are indicated in square boxes at the bottom of the plot. Statistical analysis was run separately for the samples from January and those from March on the ranked forms of all the data points present in the figure. The median populations of the groups (n=21) that share the same number before the letter but not the same letter were significantly different based on Tukey's test (P≦0.05).

FIG. 9: FIG. 9 shows the 3C populations on wheat heads closely before harvest and postharvest threshed grains. One sample of each type was randomly collected from each of the 6 inoculated and 6 non-inoculated plots at different time points. “DL” on the Y-axis represents the theoretical detection limit for this experiment. The numbers of samples that were negative for 3C detection for each time-treatment combination are indicated in the square boxes at the bottom of plot. Statistical analysis shown in this figure was run separately for different sample types and different time points on the ranked forms of all the data points present in the figure. The medians of the population groups (n=6) that share the same number before the letter but not the same letter were significantly different based on Tukey's test (P≦0.05).

FIG. 10A-10B: FIGS. 10A and 10B show the culture-based quantification of 3C population in postharvest threshed grains by colony forming units (CFU) (A) and correlation with qPCR (B). CFU quantification was performed on the grain samples of 155 days postharvest. In (A), the CFU populations groups (n=6) that do not share the same letter were significantly different based on Tukey's test (P=0.004). In (B), regression was run on inoculated samples only since none of the non-inoculated samples showed detection of 3C in qPCR (FIG. 9). “DL” on the Xaxis represents the theoretical detection limit of qPCR for this experiment.

FIG. 11: Genotypic variation among C. flavescens strains detected using multi-locus sequence typing (MLST). The MLST analysis based on the concatenated DNA sequences obtained from the genes β-tubulin, Chitin Synthase 1, Elongation Factor 1 and Heat Shock Protein 70 kDa amplified by the primers shown in Table 2. The phylogenetic relationships among taxa were inferred by the Maximum Likelihood method with General Time Reversible model. Bootstrap values of >60% (1,000 replicates) are shown next to the branches. Scale bar shows number of base substitutions per site.

FIGS. 12A-12B: Phenotypic variation among C. flavescens strains detected using Biolog assays. Cluster analysis on the carbon source utilization and chemical sensitivity patterns at early (FIG. 12A) and late (FIG. 12B) time points. Two independent runs of the assays were performed for each strain. Due to the variation in assay conditions between the two runs, the data from 7 and 12 days of incubation in Run I were approximately equivalent to those from 10 and 15 days of incubation in Run II, respectively. Cluster Variables analysis using Correlation Coefficient Distance and Ward Linkage was conducted on the OD₅₉₅ measurements. Each distinct group was defined by a similarity percentage greater than the minimum between two replicates of the same strain (*). Different bracket line patterns were used to differentiate genotype-A (homogenous dash) and genotype-B strains (alternative dash).

FIG. 13: Biocontrol efficacy of two distinct genotypes of C. flavescens. The assayed strains were 3C, Y-7373, YB-601 and YB-602 (genotype A), as well as Y-7372, YB-328, and YB-744 (genotype B). Data were collected at two time points post inoculation and pooled from five independent experiments for this analysis. A total of 69, 227, 158, and 69 heads were assayed for the negative control (NC), genotype A, and genotype B treated samples, respectively. Error bars represent standard error of the means.

FIGS. 14A-14E: C. flavescens strains consistently split into two groups, shown individually by the DNA sequences of FIG. 14A, β-tubulin, FIG. 14B, chitin synthase 1, FIG. 14C, elongation factor 1, FIG. 14D, heat shock protein 70 kDa and FIG. 14E, chitin synthase 5 plus downstream anonymous region. All the genotype-A strains were in the same clade (upper) while the genotype-B strains in the other clade (lower). The phylogenetic relationships among taxa were inferred by the Maximum Likelihood method with General Time Reversible model. Bootstrap values of >60% (1,000 replicates) are shown next to the branches. Scale bar shows number of base substitutions per site.

FIG. 15: Population dynamics of 3C-like C. flavescens on inoculated wheat heads in the field. 3C inoculum suspension was sprayed on wheat at a rate of 2×10⁶ to 6×10⁶ cells per square centimeter. The wheat head samples (n=5 to 9 depending on sample date) were collected from the three inoculated areas (see FIG. S1 for details on sampling locations) at five time points post inoculation. Tukey's test was performed on the ranked data presented in the figure, and the median populations of the groups that do not share the same letter (a, b) were significantly different (P≦0.05). “DL” on the Y-axis represents the theoretical detection limit for this experiment. Only one sample was negative for 3C detection at any measured time point and it is indicated in a square box at the bottom of the plot.

FIG. 16: Overwintering persistence of 3C-like C. flavescens in inoculated plots. Postharvest residues of the three inoculated areas were sampled in late January and late March of 2012. Composite samples (C) of soil, living weeds and brownish rotting plant materials and samples of residue wheat stalks (W) were collected within a circle of about 1-m radius around the center of each inoculated area. “DL” on the Y-axis represents the theoretical detection limit for this experiment. The numbers of samples that were negative for detection for each sample type and time point are indicated in square boxes at the bottom of the plot. Statistical analysis was run separately for the samples from January and those from March on the ranked forms of all the data points present in the figure. The median populations of the groups (n=21) that share the same number before the letter but not the same letter were significantly different based on Tukey's test of the rank transformed data (P≦0.05).

FIG. 17: Detection of 3C-like C. flavescens on wheat heads and grain pre- and post-harvest. One sample of each type was randomly collected for each of the 6 inoculated and 6 non-inoculated plots at different time points. “DL” on the Y-axis represents the theoretical detection limit for this experiment. The numbers of samples that were negative for detection for each time-treatment combination are indicated in the square boxes at the bottom of plot. Statistical analysis shown in this figure was run separately for the different sample types and different time. The medians of the population groups (n=6) that share the same number before the letter but not the same letter were significantly different based on Tukey's test of the rank transformed data (P≦0.05).

FIG. 18: Sequence alignment of the qPCR amplicons from 3C and other six C. flavescens strains. The consensus sequence is on the top of the alignment. Primer binds h31.2_F (1 to 22 bp) and h31.2_R (129 to 150 bp) as well as the region predicted by Augustus as exon (39 to 150 bp) are indicated. FIG. 18 discloses SEQ ID NOS 91-92, 92, 92, 92-94, 94-96, 96, 96, 96-97, 97, 97-99, 99, 99, 99-100, 100 and 100, respectively, in order of appearance.

### DETAILED DESCRIPTION

Throughout this disclosure, various publications, patents and published patent specifications are referenced. The disclosures of these publications, patents and published patent specifications are hereby incorporated by reference into the present disclosure to more fully describe the state of the art to which this invention pertains.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

It is to be understood that the present compositions and methods are not limited to specific synthetic methods unless otherwise specified, or to particular reagents unless otherwise specified, as these methods and reagents may vary. It is also to be understood that the terminology used herein is for the purpose of describing particular aspects only and is not intended to be limiting. Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

### 1. Definitions

As used in the specification and the appended claims, the singular forms “a,” “an” and “the” include plural referents unless the context clearly dictates otherwise. Thus, for example, reference to “a C. flavescens strain,” “an amount,” or “the plant” includes mixtures of two or more such C. flavescens strains, amounts, or plants, and the like.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if a range of 10 to 15 is disclosed, then 11, 12, 13, and 14 are also disclosed.

The term “cereal” as used herein is intended to refer to any cereal plant species that is susceptible to FHB. Cereals reported to be susceptible to FHB include, but are not limited to, wheat, barley, rice, oats, spelt, and triticale, though wheat and barley are the two crops in which this disease presents a significant economic problem. Any of these cereals can be treated for FHB using the methods described herein.

The terms “microbial antagonist” refers to microorganisms that work to prevent, suppress, treat, or control the development of a pathogen or a pathogenic disease in a plant, such as a cereal plant. A microbial antagonist can work to prevent, suppress, treat, or control a pre-harvest or post-harvest disease in plants, including their fruits and other harvestable parts. For example, a microbial antagonist can be used to treat FHB. A microbial antagonist activity can be achieved by a variety of mechanisms. In some instances, the microbial antagonist can be antagonistic to, for example, a plant pathogen, such as FHB. An antagonist can itself be a bacterium, a fungus, or other type of microorganism. For example a microbial antagonist exhibits a degree of inhibition of FHB exceeding, at a statistically significant level, that of an untreated control.

The term “suppress” or “suppressing” refers to a reduction or inhibition. For example, “suppressing Fusarium head blight” refers to reducing the level of disease caused by Fusarium head blight. “Suppressing” Fusarium head blight is interchangeable with “treating” Fusarium head blight. To treat refers to the intent to cure, ameliorate, stabilize, or prevent a disease, pathological condition, or disorder. Thus, the term covers any treatment including preventing, inhibiting, suppressing or relieving the disease.

As used herein, the terms “effective amount” and “amount effective” refer to an amount of a composition that is sufficient to achieve a desired result or to have an effect on an undesired condition. For example, the effective amount can reduce the level of Fusarium head blight relative to that in the corresponding untreated control. An effective amount is a sufficient amount to achieve a desired result without resulting in undesired effects. In one aspect of the disclosed methods, the effective amount can vary depending on factors such as, but not limited to, the type of cereal plant being treated, the quantity, the route of administration, the duration of treatment, and any compositions or microbial antagonists used in combination.

As used herein, the term “percent identity” as applied to polynucleotide sequences refers to the percentage of residue matches between at least two sequences aligned using a standardized algorithm such as the ClustalW and Muscle algorithms employed by the Molecular Evolutionary Genetics Analysis (MEGA) software (Center for Evolutionary Medicine and Informatics, Tempe, Ariz.). Other alignment tools may be used to determine the percent identity, such as the BLAST suite of programs (National Center for Biotechnology Information) (e.g., blast, blastp, blastx, nucleotide blast and protein blast) using, for example, default parameters.

Percent identity may be measured over the length of an entire defined sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

As used herein, the terms “homologue” and “homologous” when used to describe a sequence refers to a sequence that is a variant of another and wherein the two sequences are evolutionarily related. In the present disclosure, when a particular gene is referred to, the term is meant to encompass homologues and orthologues, variants, derivatives, and mutants of such a gene or protein. The present invention is not limited to embodiments employing the exact sequence of any of the disclosed polynucleotides, but encompasses any variant that is related by structure, sequence, function or is derived in any way from the named polynucleotide. For example, the present invention encompasses polynucleotides having, for example, at least 85% primary nucleic acid sequence identity among all strains of C. flavescens for genes β-tublulin, chitin synthase 1, elongation factor 1, heat shock protein 70 kDa, and cs22 (chitin synthase 5 plus downstream anonymous region).

“Variant” refers to a polynucleotide that differs from a reference polynucleotide, but retains essential properties. A typical variant of a polynucleotide differs in nucleic acid sequence from another, reference polynucleotide. Generally, differences are limited so that the sequences of the reference polynucleotide and the variant are closely similar overall and, in many regions, identical. A variant and reference polynucleotide may differ in nucleic acid sequence by one or more modifications (e.g., substitutions, additions, and/or deletions). A variant may be naturally occurring, or it may be a variant that is not known to occur naturally. A polynucleotide variant may have, for example, at least 99%, 95%, 90%, or at least 85% percent identity over the entire length of the original polynucleotide. Variants may be derivatives of the polynucleotide of which they are a variant, they may be chemically or biochemically modified and have one or more amino nucleotide substitutions, additions, and/or deletions. Variants may share certain functionally significant motifs with the polynucleotide of which they are a variant.

As used herein, the term “threshold cycle (Ct)” refers to the intersection between a qPCR amplification curve and a threshold line. The threshold line is commonly ten times the standard deviation of the background fluorescence. The first cycle which is above the threshold line is defined as the Ct. Within a certain range of template concentrations, the Ct value is proportional to the template copy number present at the beginning of the reaction and reflects the first opportunity for quantification of the template. Mismatches in the primer-probe sequences disclosed herein result in altered efficiency of DNA amplification. Generally, an increased number of mismatches leads to lowered qPCR efficiency. It is these altered qPCR efficiencies (and the underlying primer-probe mismatches) that allow for discrimination between Genotype A and Genotype B C. flavescens, as disclosed herein.

### 2. Methods of Identifying C. flavescens Genotypes

Methods of identifying specific genotypes of C. flavescens are provided. For example, Genotype A and Genotype B C. flavescens strains can be identified using the disclosed quantitative PCR or sequence identity methods. Cryptococcus is a genus of fungus that grows in culture as yeasts. Cryptococcus flavescens, a species of Cryptococcus, is an encapsulated yeast. C. flavescens is a biological control agent of FHB and has been shown to have improved desiccation tolerance. Different genotypes of C. flavescens can treat diseases, such as FHB, with different efficiencies. Therefore, it can be advantageous to develop methods of distinguishing between the C. flavescens genotypes.

Real time PCR, also referred to as quantitative PCR (qPCR), can be used to identify different species or genotypes of C. flavescens. The qPCR assay is faster, cheaper, and more reliable than the previous detection methods based solely on culturing and antibiotic resistance of the target strain. The primers and assay conditions can be readily adapted to isolate environmental strains of C. flavescens that are genetically similar to 3C and express similar biocontrol phenotypes. Furthermore, additional primer sets can be defined using the genome sequence provided by GenBank accession numbers CAUG01000001-CAUG01000712. For example, a C. flavescens Genotype A or Genotype B strain can be identified by performing qPCR on DNA obtained from a microorganism. The qPCR results in a threshold cycle value for each strain. Depending on the primer set used during qPCR, the threshold cycle value can be indicative of the specific C. flavescens genotype. Described below are examples of qPCR primers and threshold cycle values that are indicative of Genotype A and Genotype B C. flavescens strains.

Another method of identifying C. flavescens genotypes includes determining the sequence identity of specific genes. For example, determining the sequence identity of one or more of the (3-tubulin, chitin synthase 1, elongation factor 1, heat shock protein 70 kDa, and cs22 (chitin synthase 5 plus downstream anonymous region) gene sequences and comparing the determined sequence with the homologous gene sequences in known Genotype A and Genotype B C. flavescens strains can be used to identify the genotype of the strain being tested. A multilocus sequence analysis of β-tubulin, chitin synthase 1, elongation factor 1, heat shock protein 70, and cs22 (chitin synthase 5 plus downstream anonymous region) can also be used to classify C. flavescens strains into two distinct groups, or genotypes, as well. Some regions of the C. flavescens DNA, such as the Internal Transcribed Spacer (ITS) region, cannot be used to differentiate the C. flavescens subspecies because all C. flavescens strains have the same ITS sequence. Examples of sequence identity being used to determine Genotype A and Genotype B. C. flavescens strains are described below.

As described herein, threshold cycle values determined during qPCR or sequence identity can be used to differentiate the genotypes of C. flavescens. These strains can be used in the disclosed methods. The methods of identifying can include the initial step of collecting the DNA to be tested. For example, genomic DNA can be extracted from a microorganism, or cereal plant seed heads can be collected and the DNA isolated from these heads. Once the DNA has been obtained, qPCR can be performed using the primers and probes identified herein or the DNA can be sequenced, using known sequencing techniques, in order to determine the sequence identity when compared to known C. flavescens strains.

In some aspects, the methods of identifying genotypes of C. flavescens include performing qPCR with more than one primer set. The methods can also include comparing the sequence identity at more than one gene. In some instances, the methods of identifying can incorporate both the qPCR technique and the sequence identity technique for identifying a Genotype A or Genotype B C. flavescens strain. For example, qPCR using a particular primer set can be performed on DNA isolated from a microorganism and comparing the sequence identity of a particular gene of the isolated DNA to known Genotype A and Genotype B strains can also be performed. Therefore, the combination of the qPCR and the sequence identity results can provide the identity of the C. flavescens genotype.

Strains of C. flavescens in addition to those identified herein can be identified and recovered by those of skill in the art using the primers and the sequence identity described herein.

The disclosed methods can include the use of the C. flavescens strains described herein as well as compositions containing the C. flavescens strains described herein.

i. Genotype A

C. flavescens Genotype A strains can be used for the disclosed methods. Examples of Genotype A strains include OH 182.9 (3C), Y-7373, Y7377, YB601 and YB-602.

Genotype A strains can be detected by qPCR. In one aspect, amplification is performed with one or more of the primer sets selected from btub.1, btub.2, h31.2, EF1.2,h22.1, h30.2,h31.1 and 12.1 (see Table 1 and FIG. 3). The genotype A strains can have a threshold cycle value of 17-21 when using the btub.1, btub.2, or EF1.2 primer sets. The genotype A strains can have a threshold cycle value of 18-20 when using the h22.1, h30.2 or h31.1 primer sets. The genotype A strains can have a threshold cycle value of 17-20 when using the h31.2 primer set. The genotype A strains can have a threshold cycle value of 12-15 when using the I2.1 primer set.

The Genotype A strains to be used in the methods described herein can also be identified by comparing a determined polynucleotide sequence to the known homologous sequences of other Genotype A strains. For example, the percent identity of one or more of the gene sequences of (3-tubulin, chitin synthase 1, elongation factor 1, heat shock protein 70, and cs22 (chitin synthase 5 plus downstream anonymous region) compared to the disclosed sequences for these genes can be used to identify a Genotype A strain. Those strains that share the sequence identities disclosed below are considered Genotype A strains.

a. β-Tubulin Gene Sequence.

The Genotype A strains share 100% sequence identity among the strains at the β-tubulin gene. Therefore, those C. flavescens strains that have 100% identity to SEQ ID NO: 1 can be classified as a Genotype A strain.

b. Chitin Synthase 1 Gene Sequence.

The Genotype A strains can share between 95% and 100% sequence identity at the chitin synthase 1 gene. In one aspect, Genotype A strains share at least 95.2% sequence identity at the chitin synthase 1 gene. Therefore, C. flavescens strains that have between 95% to 100% sequence identity to SEQ ID NOs: 2, 3, 4, 5, or 6 can be classified as a Genotype A strain.

c. Elongation Factor 1 (EF1) Gene Sequence.

The Genotype A strains can share between 99% and 100% sequence identity at the EF1 gene. In one aspect, Genotype A strains share at least 99.7% sequence identity at the EF1 gene. C. flavescens strains that have between 99% and 100% sequence identity to SEQ ID NOs: 7, 8, 9, 10, or 11 can be classified as a Genotype A strain.

d. Heat Shock Protein 70 kDa (Hsp70) Gene Sequence

The Genotype A strains can share between 99% and 100% sequence identity at the hsp70 gene. In one aspect, Genotype A strains share at least 99% identity at the hsp70 gene. C. flavescens strains that have between 99% and 100% sequence identity to SEQ ID NOs: 12, 13, 14, 15, or 16 can be classified as a Genotype A strain.

e. Cs22 (Chitin Synthase 5 Plus Downstream Anonymous Region) Target Locus Sequence.

The Genotype A strains can share between 99% and 100% sequence identity at the cs22 target locus. In one aspect, Genotype A strains share at least 99% identity at the cs22 target locus. C. flavescens strains that have between 99% and 100% sequence identity to SEQ ID NOs: 85, 86, 87, 88, or 89 can be classified as a Genotype A strain.

ii. Genotype B

C. flavescens Genotype B, or non-3C, strains can be used for the disclosed methods. Examples of Genotype B strains include Y-7372, Y-7374, Y-7375, Y-7376, Y-7379, YB-328, and YB-744.

Genotype B strains can be detected by qPCR. In one aspect, amplification is performed with one or more of the primer sets selected from btub.1, btub.2, h31.2, EF1.2, h22.1, h30.2, h31.1 and 12.1 (see Table 1 and FIG. 3). The Genotype B strains can have a threshold cycle value of 31-33 when using the btub.1 primer set. The Genotype B strains can have a threshold cycle value of 32-35 when using the btub.2 primer set. The Genotype B strains can have a threshold cycle value of 24-26 when using the EF1.2 primer set. The Genotype B strains can have a threshold cycle value of 22-24 when using the h22.1 or h31.1 primer sets. The Genotype B strains can have a threshold cycle value of 22-23 when using the h30.2 primer set. The Genotype B strains can have a threshold cycle value of 34-35 when using the h31.2 primer set. The Genotype B strains can have a threshold cycle value of greater than 15-18 when using the I2.1 primer set.

The Genotype B strains to be used in the methods described herein can also be identified by comparing a determined polynucleotide sequence to the known homologous sequences of other Genotype B strains. For example, the percent identity of one or more of the gene sequences of (3-tubulin, chitin synthase 1, elongation factor 1, heat shock protein 70, and cs22 (chitin synthase 5 plus downstream anonymous region) compared to the disclosed sequences for these genes can be used to identify a Genotype B strain. Those strains that share the sequence identities disclosed below are considered Genotype B strains

a. β-Tubulin Gene Sequence

The Genotype B strains can share between 99% and 100% sequence identity at the (3-tubulin gene. In one aspect, Genotype B strains share at least 99.6% sequence identity at the (3-tubulin gene. C. flavescens strains that have between 99% and 100% sequence identity to SEQ ID NOs: 17, 18, 19, 20, 21, 22 or 23 can be classified as a Genotype B strain.

b. Chitin Synthase 1 Gene Sequence

The Genotype B strains can share between 98% and 100% sequence identity at the chitin synthase 1 gene. In one aspect, Genotype B strains share at least 98.8% sequence identity at the chitin synthase 1 gene. C. flavescens strains that have between 97% and 100% sequence identity to SEQ ID NOs: 24, 25, 26, 27, 28, 29 or 30 can be classified as a Genotype B strain.

c. Elongation Factor 1 Gene Sequence

The Genotype B strains can share between 99% and 100% sequence identity at the EF1 gene. In one aspect, Genotype B strains share 99.7% sequence identity at the EF1 gene. C. flavescens strains that have between 99% and 100% sequence identity to SEQ ID NOs: 31, 32, 33, 34, 35, 36, or 37 can be classified as a Genotype B strain.

d. Heat Shock Protein 70 kDa Gene Sequence

The Genotype B strains can share between 99% and 100% sequence identity at the hsp70 gene. In one aspect, Genotype B strains share 99.1% sequence identity at the hsp70 gene. C. flavescens strains that have between 99% and 100% sequence identity to SEQ ID NOs: 38, 39, 40, 41, 42, 43 or 44 can be classified as a Genotype B strain.

e. Cs22 (Chitin Synthase 5 Plus Downstream Anonymous Region) Gene Sequence.

The Genotype B strains share 100% sequence identity among the strains at the cs22 target locus. Therefore, those C. flavescens strains that have 100% identity to SEQ ID NO: 90 can be classified as a Genotype B strain.

iii. Identifying a Fungus as C. flavescens

Fungi may be identified as a strain of C. flavescens by comparing a determined polynucleotide sequence to the known homologous sequences of other C. flavescens strains. For example, the percent identity of one or more of the gene sequences of β-tubulin, chitin synthase 1, elongation factor 1, heat shock protein 70, and cs22 (chitin synthase 5 plus downstream anonymous region) compared to the disclosed sequences for these genes can be used to identify a fungus as a species of C. flavescens.

a. β-Tubulin Gene Sequence

The C. flavescens strains can share between 95% and 100% sequence identity at the β-tubulin gene. In one aspect, C. flavescens strains share at least 95.1% sequence identity at the β-tubulin gene.

b. Chitin Synthase 1 Gene Sequence

The C. flavescens strains can share between 89% and 100% sequence identity at the chitin synthase gene. In one aspect, C. flavescens strains share at least 95.5% sequence identity at the chitin synthase 1 gene.

c. Elongation Factor 1 Gene Sequence

The C. flavescens strains can share between 94% and 100% sequence identity at the elongation factor 1 gene. In one aspect, C. flavescens strains share at least 94.1% sequence identity at the elongation factor 1 gene.

d. Heat Shock Protein 70 kDa Gene Sequence

The C. flavescens strains can share between 93% and 100% sequence identity at the heat shock protein 70 gene. In one aspect, C. flavescens strains share at least 93.7% sequence identity at the heat shock protein 70 gene.

e. cs22 (Chitin Synthase 5 Plus Downstream Anonymous Region) Gene Sequence.

The C. flavescens strains can share between 79% and 100% sequence identity at the cs22 target locus. In one aspect, C. flavescens strains share at least 79.4% sequence identity at the cs22 target locus.

f. Identification of a Fungus as a Strain of C. flavescens

Across a concatenation of the genes β-tubulin, chitin synthase 1, elongation factor 1, heat shock protein 70, and cs22, C. flavescens strains share at least 89.8% sequence identity. Therefore, a fungus sharing at least 89.8% identity among these five genes may be identified as a species of C. flavescens.

### 3. Methods of Suppressing Fusarium Head Blight in Cereal Plants

Methods for suppressing FHB in a cereal plant by applying to a seed head of said plant an effective amount of at least one microbial antagonist are provided. The microbial antagonist can be a Genotype A C. flavescens strain or Genotype B Cryptococcus flavescens strain, wherein the strain of C. flavescens is not C. flavescens 3C which has been deposited under NRRL accession no. Y-50378 or C. flavescens 4C which has been deposited under NRRL accession no. Y-50379.

In one aspect of the methods, a Genotype A C. flavescens strain can be used to suppress FHB in a cereal plant by applying an effective amount of at least one Genotype A C. flavescens strain to a seed head of the cereal plant. Any of the Genotype A C. flavescens strains described herein can be used. For example, Genotype A C. flavescens strains can include Y-7373, Y7377, YB601 and YB-602.

In one aspect of the methods, a Genotype B C. flavescens strain can be used to suppress FHB in a cereal plant by applying an effective amount of at least one Genotype B C. flavescens strain to a seed head of the cereal plant. Any of the Genotype B C. flavescens strains described herein can be used. For example, Genotype B C. flavescens strains can include Y-7372, Y-7374, Y-7375, Y-7376, Y-7379, YB-328, and YB-744.

The disclosed methods involve applying an effective amount of C. flavescens to a cereal plant. An effective amount of C. flavescens can be an amount that reduces the level of FHB relative to that in a corresponding untreated control. An effective amount of C. flavescens can be from 104 to 109 CFU per ml. For example, C. flavescens levels between 107-108 CFU/ml can be used. In some instances, 5×109 CFU/ml can be applied at 20 gal/acre. In one aspect of the disclosed methods, the level of FHB in untreated controls is previously determined.

A. Fusarium Head Blight (FHB)

Head scab, also known as FHB, is a devastating disease of cereal plants, such as, wheat and barley, that is primarily caused by the fungus Gibberellazeae (anamorph=Fusarium graminearum). Fusarium graminearum primarily infects the heads (flower heads, seed heads, or spikes) of cereal plants from the time of flowering through the soft dough stage of head development. Germinated conidia or ascospores of Fusarium graminearum penetrate through anthers and associated tissues to initiate infection of the host and the development of symptoms of FHB.

The infection of seed by Fusarium graminearum reduces seed germination, seedling vigor and plant emergence [Bechtel et al., (1985) Cereal Chem. 62:191-197]. Infection of wheat kernels by Fusarium graminearum reduces grain yield and affects grain quality [Clear et al., (1990) Can. J. Plant Sci. 70:1057-1069]. In addition to causing grain yield loss, Fusarium graminearum can produce mycotoxins such as the estrogenic toxin zearalenone (F-2) (Hesseltine et al., 1978, Fungi, especially Gibberellazeae, and zearalenone occurrence in wheat. Mycologia, 70, 14-18) and the trichothecenedeoxynivalenol (DON, vomitoxin) (Snijders, 1990, Fusarium head blight and mycotoxin contamination of wheat, a review. Netherlands Journal of Plant Pathology, 96, 187-198) that can have a deleterious effect on grain quality [Cardwell et al., 2001, Mycotoxins: the cost of achieving food security and food quality, APS Net: Feature story August, 2001] and animal health [Marasas, 1991, Toxigenic Fusaria, in: Mycotoxins and Animal Foods, J. E. Smith and R. S. Henderson, eds., CRC Press, Inc., Boca Raton, Fla.; Beardall and Miller, 1994, Diseases in humans with mycotoxins as possible causes, in Mycotoxins in Grain: Compounds Other than Aflatoxin (MILLER, J. D. & TRENHOLM, H. L., Eds.). Eagan Press, St. Paul, Minn., pp. 387-39]. Therefore, there is a strong need for treating for FHB.

A common treatment of FHB comprises the application of the fungicide prothioconazole. Prothioconazole allows for an increase in grain yields and a reduction of DON in wheat kernels. However, the use of fungicides, such as prothioconazole, is becoming more and more unacceptable due to environmental concerns and possible side effects in humans or animals that consume products treated with fungicides. Although new FHB treatments may eventually replace prothioconazole, the possibility also exists that new treatments may be combined with prothioconazole treatment. Therefore, the disclosed microbial antagonists for FHB can be prothioconazole-tolerant.

B. Microbial Antagonists

The disclosed methods include the use of microbial antagonists that are strains of C. flavescens. Compositions containing the disclosed microbial antagonists are also disclosed. Although C. flavescens OH 182.9 (aka 3C) can be used to reduce FHB in cereals, it is sensitive even to low concentrations of the fungicide prothioconazole. This sensitivity has limited the application of C. flavescens against FHB in fields which are or will also be treated with prothioconazole. Variants, mutants, or other C. flavescens strains, which exhibit significantly greater tolerance to prothioconazole than the parent 3C, can be used in the disclosed methods. The prothioconazole tolerant strains can exhibit greater efficacy against FHB than the parent C. flavescens 3C strain.

Microbial antagonists useful in the methods described herein can be, but are not limited to, C. flavescens (Y-7373), C. flavescens (YB-601), C. flavescens (YB-602), C. flavescens (Y-7377), C. flavescens (Y-7372), C. flavescens (Y-7375), C. flavescens (Y-7374), C. flavescens (Y-7376), C. flavescens (YB-328), C. flavescens (Y-7379), or C. flavescens (YB-744). Other examples of C. flavescens strains can be found in U.S. Pat. No. 8,241,889. The strains described in these patents can be used in the disclosed methods to treat FHB.

The C. flavescens strains used to suppress FHB in cereal plants can be Genotype A or Genotype B C. flavescens strains. Therefore the microbial antagonists used in the disclosed methods can include the C. flavescens strains identified using the qPCR and sequence identity methods of identifying described above.

In some aspects, the microbial antagonists can be a substantially pure microorganism which is a strain of C. flavescens selected from the group consisting of Genotype A C. flavescens strain or Genotype B C. flavescens strain, wherein the strain of C. flavescens is not C. flavescens 3C which has been deposited under NRRL accession no. Y-50378 or C. flavescens 4C which has been deposited under NRRL accession no. Y-50379.

C. Cereal Plants

Cereal plants include any seed or plant that produces an edible seed, fruit or grain, including, but not limited to, oat, rye, wheat, rice, maize, sorghum, and millet. Wheat, barley, oats, spelt and triticale are known to be susceptible to FHB and can therefore be treated in the disclosed methods. Because cereal plants are consumed every day by both humans and livestock, it is critical to keep these plants from being contaminated with harmful agents, such as FHB, to the extent they cannot be used. Methods that prevent, inhibit or suppress the growth or harmful effects of agents such as FHB are considered to be methods that promote the healthy growth or life cycle of cereal plants.

D. Administration of the Microbial Antagonist

The microbial antagonists can be administered depending on a variety of factors. Administration can vary depending on factors, such as, but not limited to, stage of development of the cereal plant, weather, amount of microbial antagonist already present on the cereal plant, and the method of administration.

i. Stage of Development of Cereal Plant

The application of the microbial antagonist to the seed head can occur at any time after the boot stage and before the hard dough stage of cereal development. The cereal head can be susceptible to infection by Fusarium graminearum from the onset of flowering (anthesis) through the soft dough stage of kernel development. Thus, one time to apply the biological control agents would be from the time immediately preceding flowering until as late as the soft dough stage of kernel development. Application of antagonists to heads before flowering would allow antagonists to have colonized wheat head parts prior to the wheat head becoming susceptible to infection. Additionally, antagonists would be well positioned to colonize and protect anthers as they emerge from florets. The antagonists would still be effective if applied after flowering has begun, but before the hard dough stage of development. However, long delays may decrease the effectiveness of the treatment depending on methods of cell formulation and application. Therefore, application of the microbial antagonist can occur prior to hard dough stage of development, during flowering, or prior to flowering.

ii. Weather

The temperature at which the C. flavescens can be applied can vary. The microbial antagonists can be effective at temperatures ranging from about 5° C. to about 35° C. In one aspect of the disclosed methods, the microbial antagonists are applied at temperatures ranging from 15-30° C.

The wind or rain can also play a role in the administration of the microbial antagonists. For example, heavy wind conditions or rain may result in less microbial antagonist remaining on the cereal plant. If the wind or rain prevent some, or all, of the microbial antagonist from remaining on the cereal plant, the therapeutic effects can be minimal or non-existent.

iii. Presence of Microbial Antagonist on the Cereal Plant

Administration of microbial antagonists can also depend on whether the microbial antagonist is already present on the plant and in what quantity. Therefore, in one aspect of the methods, it may be helpful to first test the cereal plant to determine if any microbial antagonist, such as Cryptococcus flavescens is present. If C. flavescens is present, determining the quantity can be beneficial. If C. flavescens is present, but not in an amount that is considered to be an effective amount, then applying more C. flavescens to the crop may be necessary. If there is an effective amount already present on the crop then treatment with more C. flavescens can be delayed.

iv. Methods of Administration

The application of C. flavescens strains to cereal plants can vary. In one aspect, the microbial antagonists can be sprayed onto the plants. The spraying can occur through a sprinkler irrigation system or an aerial or ground-based applicator. The administration of microbial antagonists can vary depending on factors, such as, but not limited to, the size of the area receiving the antagonists and the weather. Those of ordinary skill in the art would understand how to apply microbial antagonists to cereal plants.

E. Combination Treatment

FHB can be suppressed using a combination treatment. The disclosed methods can include applying a combination of C. flavescens strains. The combination can include two or more Genotype A strains, two or more Genotype B strains or a mixture of one or more Genotype A and Genotype B strains.

In some aspects of the disclosed methods, the microbial antagonist can include a Genotype A or Genotype B C. flavescens strain in combination with C. flavescens 3C which has been deposited under NRRL accession no. Y-50378 or C. flavescens 4C which has been deposited under NRRL accession no. Y-50379. Thus, in one aspect, at least one of the C. flavescens strains is C. flavescens 3C or C. flavescens 4C and at least a one strain is not C. flavescens 3C or C. flavescens 4C.

A combination treatment can also include treating with a fungicide, such as prothioconazole, as well as a C. flavescens strain. Any of the disclosed C. flavescens strains can be used in the combination treatments. The order of the treatment can vary. In one instance, cereal plants are first treated with prothioconazole and then treated with one or more C. flavescens strains. In one instance the prothioconazole and C. flavescens strains are applied simultaneously. In yet another instance, the prothioconazole is applied after treatment with C. flavescens.

In certain aspects, cereal plants are treated with a first C. flavescens strain and then treated with one or more additional C. flavescens strains. In one aspect, the first C. flavescens strain is applied simultaneously with the additional C. flavescens strains. In another aspect, the additional one or more C. flavescens strains are applied in one or more applications subsequent to the application of the first C. flavescens strain.

### 4. Methods of Determining the Presence of C. flavescens on a Cereal Plant

Methods of determining the presence of C. flavescens, particularly a Genotype A or Genotype B C. flavescens strain, on a cereal plant are provided. Any of the disclosed qPCR methods or primers can be used to identify the presence of Genotype A or Genotype B C. flavescens strains. For example, qPCR can be performed on a sample taken from a cereal plant in the field. Using the same threshold cycle value analysis developed for identifying specific C. flavescens genotypes, the presence of Genotype A or Genotype B strains can be determined.

In one aspect, at least one Genotype A strain and at least one Genotype B strain are each present. If only a Genotype B strain is determined to be present on the cereal plant, then a Genotype A strain can be administered to the cereal plant. Of course, the presence of a C. flavescens strain alone may not be the only factor involved in determining what, if any, treatments are needed on the cereal plant. The quantity of the C. flavescens can also play a role. Thus, if there is not an effective amount of the C. flavescens present, then administration of a Genotype A or Genotype B strain may be necessary.

The presence of a Genotype A or Genotype B C. flavescens strain can also be determined based on the methods described herein regarding using the sequence identity of one or more particular genes in the sample compared to the gene sequence of a known Genotype A or B C. flavescens strains.

The method of determining the presence of a Genotype A or Genotype B C. flavescens strain can be performed before or after administration of one of the disclosed microbial antagonists. In one aspect of the methods, the presence of the C. flavescens strains can be determined both prior to and after administration of a microbial antagonist.

Therefore, the methods can be used to verify the presence of a microbial antagonist on the cereal plant. In one aspect of the methods, after determining the presence of a Genotype A C. flavescens strain, either more Genotype A strain can be administered or a Genotype B strain can be administered. In some instances, a combination of C. flavescens genotypes can be administered.

The methods of determining or verifying the presence of C. flavescens can include collecting a cereal plant seed head sample. Collecting the sample can be performed by an individual or a machine or device used in the field. Once the sample has been collected, the DNA can be harvested from the cereal plant and qPCR or sequencing described herein can be performed so that the threshold cycle value or the sequence identity to known C. flavescens genotypes can be used to determine the presence of a Genotype A or Genotype B C. flavescens strain.

Because certain strains and genotypes of C. flavescens have more beneficial effects on treating FHB than other strains, there is a need for determining the presence of C. flavescens on cereal plants, especially determining the genotype of the C. flavescens.

In one aspect of the methods, the presence of a fungicide, such as prothioconazole, is determined. If prothioconazole is determined to be present then using a prothioconazole tolerant strain of C. flavescens to treat the cereal plant can be necessary.

### EXAMPLES

The following examples are put forth so as to provide those of ordinary skill in the art with a complete disclosure and description of how the compounds, compositions, and methods claimed herein are used and evaluated and are intended to be purely exemplary of the invention and are not intended to limit the scope of what the inventors regard as their invention. However, those of skill in the art should, in light of the present disclosure, appreciate that many changes can be made in the specific aspects which are disclosed and still obtain a like or similar result without departing from the spirit and scope of the invention. Unless indicated otherwise, parts are parts by weight, temperature is in ° C. or is at ambient temperature, and pressure is at or near atmospheric.

### Example 1

### Two Distinct Subpopulations of C. flavescens: Genotype A and Genotype B

Materials and Methods

Quantitative PCR (qPCR). Each qPCR reaction had a total volume of 25 μl, consisting of 2.5 μl template DNA, 0.4 pmol/μl of each primer (forward primer h31.2_F: 5′-CGTCAGCGTGTTGCCACTTCGT-3′ (SEQ ID NO: 67); reverse primer h31.2R: 5′-GCTGCTGTCTTGCGGTCGCTTA-3′ (SEQ ID NO: 68)), 12.5 μl iQ™ SYBR® Green Supermix (Bio-Rad Laboratories, Inc., California, USA) and template-free PCR water as the rest of the volume. Template-free PCR water was also used as the template of negative controls. The thermal cycling program was run on the iCycler and the fluorescence data was collected by the iQ™5 detection system (Bio-Rad Laboratories, Inc.). Amplification was two-step with 3 min at 95° C. followed by 40 cycles of 15 s at 95° C. and 1 min at 72° C. Then the melting curve was run from 55° C. to 95° C. with an increment of 0.5° C. and a dwelling time of 10 s at each temperature. The raw data of qPCR was analyzed using the iQ™5 Optical System Software (Bio-Rad Laboratories, Inc.).

Results

Specific qPCR primers can be used to subtype C. flavescens strains into two genotypes, A and B. The primers designed to test the C. flavescens strains are shown in Table 1. Of the 12 sets tested against 6 different C. flavescens strains, EF1.1 and 5I could not distinguish any variation in vitro. However, primer sets btub.1, btub.2 and h31.2 did distinguish 2 different genotypes by differences of 11 to 14 in threshold cycle value. Primer sets EF1.2, h22.1, h30.2, h31.1, and 12.1 also distinguished 2 different genotypes by differences of 1 to 3 in threshold cycle value.

TABLE 1
Primer sets for C. flavescens.

| | | | | | Relative to Exon (E) | |
|---|---|---|---|---|---|---|
| | | | Tm (° C.) | | and/or Intron (I) | |
| | Primer | | Geneious | Eurofins | Primer | Amplicon |
| Taget Gene | Name | Sequence (5′→3′) | (Primer3) | MWG O.. | location | span (F→R) |
| btub @ | btub.1_F | TGCTTCCACGCTGTGATGCCAC | 60.24 | 66.4 | I | I→E |
| NODE_610 | | (SEQ ID NO: 45) | | | | 102 bp |
| | btub.1_R | TGTACGAACCGTCGGCCTGGAT | 59.99 | 66.4 | E | |
| | | (SEQ ID NO: 46) | | | | |
| | btub.2_F | TGCGTGCGCGGTTCCATCATAC | 60.43 | 66.4 | I | I→E |
| | | (SEQ ID NO: 47) | | | | 134 bp |
| | btub.2_R | AAACCCTGGAGGCAGTCCGTGT | 60.05 | 66.4 | E | |
| | | (SEQ ID NO: 48) | | | | |
| Chitin | CS.1_F | TCAACACGCTTCCGCCCTCAAC | 59.93 | 66.4 | E | E |
| synthase 5 | | (SEQ ID NO: 49) | | | | 145 bp |
| @ | CS.1_R | GCCTGAAGCCCGGATTTGCGAT | 60.11 | 66.4 | E | |
| NODE_22 | | (SEQ ID NO: 50) | | | | |
| | CS.2_F | GCGTGGCGGGAGAAATTGACCA | 59.99 | 66.4 | E | E |
| | | (SEQ ID NO: 51) | | | | 138 bp |
| | CS.2_R | GTTGTGACTGGCAAGCTCCGCA | 60.18 | 66.4 | E | |
| | | (SEQ ID NO: 52) | | | | |
| EF1 2 | EF1.1_F | TGACGCCGCCATTGTCAAGCTC | 60.30 | 66.4 | E | E |
| NODE_50 | | (SEQ ID NO: 53) | | | | 112 |
| | EF1.1_R | GGCAACGGTCTGTCGCATGTCT | 59.74 | 66.4 | E | |
| | | (SEQ ID NO: 54) | | | | |
| | EF1.2_F | TCGTGTACGTCCTTGCAGACGC | 59.50 | 66.4 | I | I→E→I |
| | | (SEQ ID NO: 55) | | | | 115 bp |
| | EF1.2_R | AAGGCGGGACTAACCTGCTCGT | 59.92 | 66.4 | E→I | |
| | | (SEQ ID NO: 56) | | | | |
| hsp70 @ | h22.1_F | GACTTCAACGACGCCCAGCGAT | 59.81 | 66.4 | I(1b)→E | I(1b)→E |
| NODE_227 | | (SEQ ID NO: 57) | | | | 146 bp |
| | h22.1_R | ACGATGATCTGCGACTCGGCCT | 60.11 | 66.4 | E | |
| | | (SEQ ID NO: 58) | | | | |
| | h22.2_F | AGAGTCCATCCGCGCAGTGCTA | 60.05 | 66.4 | I | I→E→I |
| | | (SEQ ID NO: 59) | | | | 146 bp |
| | h22.2_R | TCTTGCCACTCACAGGCCGGAA | 60.37 | 66.4 | E→I | |
| | | (SEQ ID NO: 60) | | | | |
| hsp70 @ | h30.1_F | AGGGCGAGCGACTGATTGGTGA | 60.24 | 66.4 | E | E→I→E |
| NODE_301 | | (SEQ ID NO: 61) | | | | 120 bp |
| | h30.1_R | TCGAAGACGGTGTTGGTCGGGT | 60.11 | 66.4 | E | |
| | | (SEQ ID NO: 62) | | | | |
| | h30.2_F | ACCAAGGACGCTGGTGCGATTG | 59.99 | 66.4 | E | E |
| | | (SEQ ID NO: 63) | | | | 112 bp |
| | h30.2_R | CGTTTCGCTCCTCGCTCGTCTT | 59.56 | 66.4 | E | |
| | | (SEQ ID NO: 64) | | | | |
| hsp70 @ | h31.1_F | CGGCTTACCGCTTGGAGGCAAA | 59.99 | 66.4 | I→E | I→E→I |
| NODE_318 | | (SEQ ID NO: 65) | | | | 114 bp |
| | h31.1_R | ACGAAGTGGCAACACGCTGACG | 60.49 | 66.4 | I | |
| | | (SEQ ID NO: 66) | | | | |
| | h31.2_F | CGTCAGCGTGTTGCCACTTCGT | 60.49 | 66.4 | I | I→E |
| | | (SEQ ID NO: 67) | | | | 150 bp |
| | h31.2_R | GCTGCTGTCTTGCGGTCGCTTA | 60.05 | 66.4 | E | |
| | | (SEQ ID NO: 68) | | | | |
| 5.8S + ITS2 | 5I.1_F | TCTTTGAACGCACCTTGCGCCT | 59.86 | 66.5 | 5.8S | 5.8S→ITS2 |
| @ | | (SEQ ID NO: 69) | | | | 120 bp |
| NODE_197 | 5I.1_R | TGCTGGCAGACACCCAAGTCCA | 60.31 | 66.4 | ITS2 | |
| | | (SEQ ID NO: 70) | | | | |
| ITS @ | I2.1_F | AGGCTTGGACTTGGGTGTCTGC | 59.02 | 66.4 | ITS2 | ITS2 |
| NODE_197 | | (SEQ ID NO: 71) | | | | 136 bp |
| | I2.1_R | AAGGTGCGCGATGGCAGGTTT | 59.91 | 64.5 | ITS2 | |
| | | (SEQ ID NO: 72) | | | | |

The amplification curves of qPCR using the primers from Table 1 are shown in Figures. 3A-3J. These curves show the differences in threshold cycle value displayed by different genotypes.

The primers and target loci used for the multilocus sequence typing analysis (MLST) are shown in tables 2 and 3, respectively. All C. flavescens isolates were found to have ITS sequences (covering the ITS1+2 regions of the rRNA gene) identical to strain 3C and distinct from C. neoformans (FIG. 1A). MLST of the β-tubulin, chitin synthase, heat shock protein 70 and elongation factor 1 genes showed two distinct genotypes of C. flavescens (FIG. 1B). The percent identity among strains using MLST is shown in Table 3.

TABLE 2
Primers and target loci for the MLST analysis on C. flavescens strains.

| | | | Number of bp^{a} | | Exon/Intron^{b} | | |
|---|---|---|---|---|---|---|---|
| Primer | | Target | ampli- | used in | Num- | Total | |
| Name | Sequence (5′→3′) | locus | fied | MLST | ber | bp | Reference |
| ITS4 | TCCTCCGCTTATTGATATGC | ITS1- | 553 | 531 | 1/2 | 156/375 | White et |
| | (SEQ ID NO: 73) | 5.8S- | | | | | al. 1990 |
| ITS5 | GGAAGTAAAAGTCGTAACAAGG | ITS2 | | | | | |
| | (SEQ ID NO: 74) | | | | | | |
| btb.m1-2F | GGGCGGTCTTGATTGACTTGGAACC | β- | 585 | 534 | 4/3 | 379/155 | This study |
| | (SEQ ID NO: 75) | tubulin | | | | | |
| btb.m2R | TCGTTGTCGATACAGAAGGTCGCGT | | | | | | |
| | (SEQ ID NO: 76) | | | | | | |
| CS24.m2F | ACAGGCTTGGAGGGAGAAGATCACG | Chitin | 356 | 332 | 2/1 | 229/103 | |
| | (SEQ ID NO: 77) | Syn- | | | | | |
| CS24.m2R | CGAACGAGGTAGAATCAGGCTTCGC | thase | | | | | |
| | (SEQ ID NO: 78) | 1 | | | | | |
| EF1.m2F | CGTGGTACAAGGGATGGACCAAGGA | Elon- | 658 | 591 | 3/2 | 413/178 | |
| | (SEQ ID NO: 79) | gation | | | | | |
| EF1.m2R | CAATGTGGGCAGTGTGGCAGTCAAG | Factor | | | | | |
| | (SEQ ID NO: 80) | 1 | | | | | |
| h31.m1F | ATGTACGCCGCTACCCGATCTCTTC | Heat | 719 | 698 | 5/4 | 508/190 | |
| | (SEQ ID NO: 81) | Shock | | | | | |
| h31.m1R | CGACAAAGGCACCAATTTGCGATGG | Protein | | | | | |
| | (SEQ ID NO: 82) | 70 kDa | | | | | |

^{a}The numbers were approximate since the minority of the strains have insertion or deletion (less than 1% of the total numbers).
^{b}The 5.8S and ITS regions were counted here as exon and introns, respectively.

Two distinct genotypes of Cryptococcus flavescens can be identified using the primer sets described herein. Genotype A is defined by the type strain OH 182.9 (3C), as well as isolates Y-7273, YB601 and YB-602, all of which are readily detected by the qPCR assay (FIG. 2). Genotype B is defined by isolates Y-7272, YB-328, and YB-744, all of which can only be detected after approximately 34 cycles using the qPCR assay described herein (FIG. 2). Additional primer sets tested during the development of the qPCR assay also revealed the same genotypic distinctions among strains. In total, these data clearly indicate two phylogenetically distinct lineages of C. flavescens among the isolates in the USDA collection.

The sensitivity and specificity of the qPCR assay was determined. The primer pair h31.2 used in the qPCR assay were designed to amplify part of a putative Heat Shock Protein 70 kDa (Hsp70) gene identified from the genomic sequence of 3C identified by accession numbers CAUG01000001-CAUG01000712. The target region was 150 bp and includes an exon-intron conjunction. The products of selected qPCR reactions were run on an electrophoresis gel to show the banding patterns (FIG. 2). The 150-bp target region of 3C gave rise to a clear band. The target bands of the standard series displayed a gradient of decreasing darkness from template concentration 1 to 1×10⁻⁴ ng/μl, while there was no visible band from 1×10⁻⁵ to 1×10⁻⁷ ng/μl. Since the theoretical detection limit was 0.8×10⁻⁵ ng/μl 3C genomic DNA in template (equivalent to one copy of target gene per reaction), it is reasonable that no clear band was observed when the template concentration of 3C genomic DNA was 1×10⁻⁵ ng/μl or less.

TABLE 3
DNA sequence identities among C. flavescens strains.

| | % identity^{a} among . . . | | |
|---|---|---|---|
| Target Locus | 3C type^{b} | non-3C type^{b} | all the strains |
| ITS1-5.8S-ITS2 | 100 | 100 | 100 |
| β-tubulin | 100 | 99.6 | 95.1 |
| Chitin Synthase 1 | 95.2 | 97.3 | 87.7 |
| Elongation Factor 1 | 99.7 | 99.5 | 93.6 |
| Heat Shock Protein 70 kDa | 99.0 | 99.1 | 93.7 |
| Concatenation of the four | 99.8 | 99.1 | 93.1 |
| protein-encoding genes above | | | |

^{a}The percentage of columns in the alignment for which all the sequences were identical.
^{b}The strains classified as 3C type are 3C, Y-7373, Y-7377, YB-601 and YB-602; those classified as non-3C type are Y-7372, Y-7374, Y-7375, Y-7376, Y-7379, YB-328 and YB-744.

There was also a lighter band of slightly >200 bp than the expected target region of 150 bp (FIG. 2). This lighter band might be another region in 3C genome that has sequence similarities to the primers but are not perfect matches. The co-occurrence and co-absence of this less specific band with the target 150-bp band in the reactions using 3C genomic DNA or PCR water as templates (FIG. 2) ruled out the possibility of it being a contaminating band from other organisms than 3C. The actual starting quantity of the target region might be slightly lower than what was demonstrated as the qPCR output because of this less specific band. However, this systematic error should be minimal since the band was even fainter than the 150-bp band in the reaction where the threshold cycle (Ct) value was 35.0 (FIG. 2). According to the regression equation, this Ct value is equivalent to 2.23×10⁻⁵ ng/μl 3C genomic DNA in template or 7.43×10³ target gene copies per gram wheat head tissue, which is at the similar magnitude to the theoretical detection limit. Therefore this less specific band should not be of significant interference to the quantification of this qPCR assay.

To test the specificity of this assay within the Cryptococcus flavescens species, qPCR was also run on the 1 ng/μl genomic DNA templates of six other C. flavescens strains than 3C (FIG. 2). Strains Y-7372, YB-328 and YB-744 showed much higher Ct values than 3C genomic DNA at the same template concentration. The Ct difference of around 15, which equals to approximately 3×10⁴-fold difference in starting quantity, between 3C and the first three strains can well differentiate 3C from them. However, strains Y-7373, YB-601 and YB-602 cannot be differentiated from 3C by this assay since they showed comparable Ct values to 3C.

### Example 2

### Different Levels of Efficacy of the Two Genotypes of C. flavescens to Control Fusarium Head Blight

The ability of genotype A and genotype B of C. flavenscens to suppress FHB on wheat was assessed in five separate greenhouse bioassays. Reduction in disease severity caused by pre-inoculation with C. flavescens strains were observed in four of five independent trials. In two of those trials, the observed pattern was statistically significant (P<0.10 by the K-W test) after 16 days of incubation. In total, the Genotype A strains displayed greater biocontrol activity than the B genotype strains in three of five trials.

Significant reductions in disease severity due to inoculation by C. flavescens were observed at 10 and 16 days post-inoculation (P=0.11 and 0.03, respectively, by the K-W test; FIG. 4). Most significantly, the A genotype reduced disease severity relative to the negative control after 16 days of incubation (P<0.10). The B genotype also appeared to reduce disease severity, but the pattern was not statistically significant. As a group, the A genotypes reduced average disease severity at 10 days (16% vs 23% for the negative control) and 16 days (46% vs 64% for the negative control) post inoculation. The B genotypes reduced average disease severity (to 17% and 51% at 10 and 16 days, respectively), but not so dramatically as the A genotypes. These data demonstrate that both genotypes of C. flavescens have the capacity to control FHB, and of these, the A genotype is more effective at controlling FHB than the B genotype.

### Example 3

### Environmental Monitoring of Cryptococcus flavescens Strains with Biological Control Activity Against Fusarium Diseases

Materials and Methods

Strains. Cryptococcus flavescens strain OH 182.9 (3C) was obtained from the Northern Regional Research Laboratory (NRRL) under accession number NRRL Y-30216. Six other C. flavescens strains were also obtained from NRRL under accession numbers Y-7372, Y-7373, YB-328, YB-601, YB-602 and YB-744, respectively. All the strains were maintained as glycerol stocks at −80° C. for long term storage.

Field establishment and 3C application. The experiments of this study were performed in two fields planted with Soft red winter wheat (Triticumaestivum L.) at the Snyder Farm at the northern end of Wooster, Ohio. The first field, subsequently noted as Field One, was planted with Cultivar Hopewell on Oct. 11, 2010. The layout of Field One is shown in FIG. 5. The second field, subsequently noted as Field Two, was planted with Cultivar Freedom on Oct. 6, 2011. Wheat in Field Two was planted in a rectangle area of 85 m×16 m containing 14×7 plots of 4.6 m×1.7 m per plot with spaces of 1.5 m along the width and 0.6 m along the length. The data shown in FIGS. 2 and 6-9 were collected from Field One and Two, respectively.

Log-phase cells of 3C produced in SDCL medium served as a 5% seed inoculum for the production in B Braun D-100 fermentor charged with 80 liters of SDCL medium. The fermentor was operated at 25° C., 20 liter/min aeration and 200 revolution/min with agitation provided by twin Rushton impellers. Eighteen hours after inoculation, fermentor temperature was reduced to 15° C. to cold shock the cells for 24 h prior to harvest. The cells were harvested through concentration into a paste using a Sharples 12-V tubular bowl centrifuge. The cell paste was then suspended in weak PO₄ buffer at a concentration of approximately 2×10⁹ CFU/ml and frozen at −20° C. as stock. The field inoculum was prepared right before application by mixing 1.5 liter of stock (pre-thawed at 10° C. overnight) with 1.5 liter sterile double distilled water and 10.8 ml 10% Tween 80. During anthesis of the wheat (Feekes Stage 10.5; on May 29, 2011), three areas of 6.1 m by 6.1 m (Plots i_{—}1, i_{—}2, and i_{—}3 of FIG. S1) were sprayed with the 3C inoculum of approximately 1.5×10⁹ cells/ml at the rate of 2×10⁶ to 6×10⁶ cells per square centimeter.

The method of producing 3C inoculum for Field Two was similar to that in Schisler et al. (Plant Disease, 2002). Briefly, 500-ml flasks containing 200 ml SDCL medium inoculated with 3C (starting OD of 0.10 at A620) were incubated at 25° C. at 250 revolution/min for 24 h. These cultures were then used to seed 3-L Fernbach flasks containing 1.25 L of SDCL medium to an OD of 0.10. Cultures were incubated for 48 h at 25° C. and 250 revolution/min. Colonized broth of 2.3×10⁸ CFU/ml was then transferred into sterile containers, transported to the field on ice and applied at the rate of 4.4×10⁵ CFU per square centimeter in the field within 24 h. The application was performed during anthesis of the wheat (Feekes Stage 10.5; on May 29, 2012). Sampling for this study was performed in 12 plots, 6 of which were sprayed with 3C only and another 6 of which were not sprayed with any microorganism. Some other plots in the field were also sprayed with 3C but they were all sprayed with other microorganism as well. The plot locations of different treatments were random designed across the field.

Sample collection. Wheat head samples were collected by clipping at the stalk 1 to 2 cm below the head and sealing each head in an individual sample bag. During sample collection, each head was only touched with the inside of the bag used to collect it and the clipping scissors were sterilized with 75% aqueous ethanol to prevent cross-contamination. Heads were collected from Field One immediately after the spray application of 3C (0 day post inoculation) as well as 24 h (1 day), 10, 26 and 44 days post inoculation in the summer of 2011 (see FIG. 5 for details on the locations of sampling). Heads were also collected from Field Two on 11 day and 1 day prior to harvest in the summer of 2012, where two heads were collected respectively from two random locations within each of the 12 plots at each time point.

Postharvest residue samples were collected from the three inoculated areas of Field One in late January and late March of 2012. Seven different locations were sampled within a circle of about 1-m radius around the center of each inoculated area. At each time point, two types of samples were collected from each location: a combination of soil, living weeds and brownish rotting plant materials was collected as one composite sample, and non-rotting wheat stalks were collected as the other sample. The grains were harvested from each plot as a whole for the 12 plots of Field Two on Jun. 26, 2012. The total grains of each plot were stored in one paper bag as a layer of thinner than 6 cm at dark at room temperature. One subsample of approximately 10 g was collected from the total grains of each plot immediately prior to grinding up for DNA extraction at 14, 64 and 155 days postharvest, respectively.

Sample handling, DNA extraction, and template preparation. Genomic DNA of 3C and the six other C. flavescens strains was extracted from 2-day old cultures grown on ⅕ Tryptic Soy Agar using PowerSoil™ DNA Isolation Kit (MO BIO Laboratories, Inc., California, USA) following the manufacturer's instruction. These original DNA extracts were diluted by more than 20-fold to certain concentrations based on the measurements on NanoDrop ND-1000 (Thermo Scientific, Delaware, USA) to prepare the templates for qPCR.

The wheat head and stalk samples were returned to the lab within 1 hr of sampling and stored at −80° C. The grain samples were stored at room temperature after being brought back from the field. These wheat samples were ground up in liquid nitrogen immediately prior to DNA extraction. The composite residue samples were stored at 10° C. after being brought back to the lab and then DNA extraction was done within 24 h directly from the samples without grinding them up. Total DNA was extracted from approximately 0.3 gram of each sample using PowerSoil™ DNA Isolation Kit following the manufacturer's instruction, yielding 100 μl DNA extract per sample. Since the DNA concentrations of the wheat head and grain samples were so high (due to the abundant wheat DNA from fresh tissue) as to interfere with the interpretation of qPCR data, 1:20 dilutions of the original DNA extracts were used as templates for all those samples. Non-diluted DNA extracts were used as the templates of postharvest residue samples from Field One.

Quantitative PCR (qPCR). qPCR was performed as described in Example 1.

Culture-based quantification. Grain samples from Field Two at 155 days postharvest were quantified for 3C population using a dilution plating method. 1±0.01 g grains from each of the 12 bags (respectively for 12 plots, 6 inoculated and 6 non-inoculated) were transferred using a sterile spoon into a sterile polypropylene tube with a capacity of 15 ml. 2 ml sterile aqueous solution containing 10 mM K₂HPO₄, 10 mM KH₂PO₄ and 0.04% Tween 80 (Sigma-Aldrich, Missouri, USA) was added to each tube. The tubes were vortexed for 20 sec, shaken at room temperature at 200 revolution/min for 10 min and then vortexed again for 20 sec. 200 μl of resulting suspensions and their 10-fold and 100-fold dilutions was spread on each plate of ⅕-strength Tryptic Soy Agar amended with 1 ppm Prosaro® fungicide (Bayer CropScience LP, North Carolina, USA), 50 ppm Streptomycin, 50 ppm Kanamycin and 50 ppm Rifampicin (Sigma-Aldrich). 3C populations were enumerated as colony forming units (CFU) of yeast-like morphology on the plates after 5 days of incubation at 25° C.

Data processing and analysis. The standard series of 1, 1×10⁻¹, 1×10⁻² . . . 1×10⁻⁷ ng/μl 3C genomic DNA in template were run in triplicate to obtain the regression equation Y=5.792−0.2984X. This equation was used to calculate from the field samples' threshold cycle values (X) their log starting quantity values (Y) as equivalences to 3C genomic DNA concentration in template. The unit of starting quantity was converted from “ng/μl 3C genomic DNA in template” to “target gene copy number per gram sample. Unit conversion was based on the following two assumptions: (i) there was one copy of target gene per genome, supported by BLAST search of this gene and the h31.2 primers against the genome of 3C (accession numbers CAUG01000001-CAUG01000712) and by the sequencing coverage of the respective contig relative to the coverage of the whole genome; (ii) the genome size of 3C was about 0.02 pg per haploid genome (estimated based on the genome sizes of Cryptococcus gattii and C. neoformans) (ref. Kullman, B., Tamm, H. & Kullman, K. 2005. Fungal Genome Size Database. http://www.zbi.ee/fungal-genomesize). Accordingly, 1 ng 3C genomic DNA was considered equal to 5×10⁴ copies of target gene and, reciprocally, one copy of target gene equal to 2×10⁻⁵ ng 3C genomic DNA. In this study, each reaction started with 2.5 μl template, which belonged to a total of 100 μl DNA extract resulted from 0.3 gram sample. Therefore, 1 ng/μ13C genomic DNA was considered equal to 3.3×10⁸ (108.5) copies of target gene per gram sample for the wheat head and grain samples (1:20 dilutions of original DNA extracts as templates) and 1.7×10⁷ (107.2) copies per gram sample for postharvest residue samples from Field One (non-diluted DNA extracts as templates).

The theoretical limit of detection for any given PCR reaction was considered to be one copy of target gene per reaction. Given the two aforementioned assumptions for unit conversion and that each reaction started with 2.5 μl template in this study, the theoretical detection limit of this study was 2×10⁻⁵ ng 3C genomic DNA per 2.5 μl, equal to 8×10⁻⁶ ng/μ13C genomic DNA in template. For the field samples, of which a total of 100 μl DNA extract resulted from 0.3 gram sample, the theoretical detection limits were 2.7×10³ (103.4) copies of target gene per gram wheat head tissue for the wheat head and grain samples and 1.3×10² (102.1) copies per gram sample for the postharvest residue samples from Field One.

In the analysis on qPCR data, only the samples where a single peak of 87.0 or 87.5° C. (melting temperature of the standard series) was the only peak recognized by the iQ™5 detection system were considered as positive for detection of 3C. The samples showing no peak of 87.0 or 87.5° C. were considered as no detection of 3C and their Threshold Cycle (Ct) values were considered as 41 for statistical analysis. The samples showing a peak of 87.0 or 87.5° C. as well as other peaks were trimmed out of further analysis since they showed detection of 3C but the quantification will not be precise enough due to the interference of non-specific peaks. A few statistical outliers were also trimmed out. For each experiment, the trimmed-out data points never exceeded 7% of the total data points, unless specified. Analysis of variance using General Linear Model where α=0.05 was performed on the ranked forms of Ct values using the software Minitab 16 (Minitab Inc., Pennsylvania, USA).

Results

Dynamics of 3C Population in Artificially Inoculated Areas.

The population density of 3C on wheat heads, represented by the target gene copy number per gram wheat head tissue, was fluctuating over time in the areas artificially inoculated with 3C (FIG. 6). The samples at one day post inoculation (DPI) showed significantly lower 3C population density than those at and after 10 DPI. There was an obvious trend that 3C population density decreased within the day immediately after artificial inoculation and then increased during between 1 and 10 DPI. This indicates the DNA of 3C applied to the wheat heads was partially degraded after artificial inoculation, but the surviving 3C cells also started to multiply on wheat heads, resulting in the growth of 3C population later on. There was a slight trend of progressive increase in 3C population density from 10 to 26 and to 44 DPI but the increase was smaller than that from 1 to 10 DPI. This indicates that later in the season the 3C population was still growing but the growth rate was reduced. This is probably due to the saturation of non-senescing wheat head tissue as colonization niche for 3C by microorganisms and/or the senesce of wheat lowering the colonization capacity for 3C later in the season.

3C population density on majority of the samples was between 105.5 and 106.0 copies of target gene per gram wheat head tissue immediately post inoculation (0 DPI), decreased to between 104.5 and 105.5 one day later and then gradually increased to between 106.0 and 106.5 by the end of the season. Given that the inoculation rate was 106.3 to 106.8 cells per square centimeter and that the horizontal section of a wheat head was around one square centimeter, the 3C population density was maintained at a level that was lower than but still comparable to the inoculation rate in the field during the growing season.

Spread of 3C to the Non-Inoculated Area of Field.

There was a trend that the 3C population density decreased as the distance from inoculated area increases and that the 3C population density in non-inoculated area increased over time from 1 to 44 DPI (FIGS. 7A and 7B). This indicates that 3C was spread stochastically instead of chaotically from artificially inoculated area to non-inoculated area over time. By the end of the growing season (44 DPI), the majority of samples from non-inoculated area were detected as 3.3×10³ to 3.3×10⁵ target gene copies per gram wheat head.

There were a few samples in non-inoculated area on 0 DPI that were detected as 3.3×10² to 3.3×10³ target gene copies per gram wheat head, while all the non-inoculated samples on 1 DPI showed no detection of 3C. However, this should not be considered as strong evidence that 3C population density was higher in non-inoculated area on 0 DPI than on 1 DPI, since those few 0-DPI samples were merely around the theoretical detection limit (2.7×10³ target gene copies per gram wheat head).

The Effect of Sample Collection Activity on 3C Spreading.

To test if sample collectors walking throughout the field contributed to the spreading of 3C, additional wheat head samples were collected from borders of wheat-grown area in Field One on 10 DPI. Since sample collectors walked along the fallow walkways in between the wheat-grown area, it was possible that they brought 3C from artificially inoculated area to noninoculated area. It was hypothesized that if sample collection activity did contribute to 3C spreading, the wheat heads at the borders of wheat-grown area neighboring the walkways should show higher 3C population density than those deeper inside of wheat-grown area do. The samples relevant to this hypothesis were collected from the eastern side of the field (see FIG. 5 for details on sampling locations) since the sample collectors were asked to exit the field from that side after they collected samples from inoculated area. Tukey's test was run on the ranked data for this experiment. The 23 samples collected from the 12 border locations (two samples per location; one data point trimmed due to non-specific amplification) near the inside locations indicated by Roman numeral II (location Group II) were compared to the 7 samples of location Group II (P=1.00). The 10 samples collected from the 12 border locations (one samples per location; two data points trimmed due to non-specific amplification) near the locations of Group III were compared to the 5 samples of location Group III (P=0.78). Therefore there was no significant difference between the 3C population densities on the wheat heads from the borders and inside of wheat-grown area, which indicates that the sample collection activity did not significantly contribute to the spreading of 3C in this field.

The Effect of Prevailing Wind Direction on 3C Spreading.

To test if the direction of prevailing wind (from west to east) affected the patterns of 3C spreading, the wheat head samples from the western and eastern sides of Field One were compared. The samples from different location groups (see FIG. 5 for details on location groups) and/or at different time points were compared separately. It was hypothesized that the samples from the eastern side of the field should show higher 3C population density than those from the western side do if the prevailing wind did play an important role in 3C spreading. Actually the differential patterns of 3C population density were not consistent across different location groups and sampling time points and there was no obvious trend towards either side of the field. This indicates that the direction of prevailing wind did not have a significant effect on the patterns of 3C spreading.

Overwintering Persistence of 3C.

To test if 3C can persist in the field over the winter, postharvest residues in the three inoculated areas of Field One were sampled in the winter to early spring of 2012. The data showed that 3C could still be detected in inoculated area by 6 to 9 months postharvest (FIG. 8). Since there was no significant difference among the same type of samples in different inoculated areas (data not shown), the data points from the three inoculated areas were combined for the comparison between sample types. The non-rotting wheat stalk samples showed significantly higher levels of 3C population (mostly 104 to 105) per gram weight than the composite samples of soil, living weeds and rotting plant materials (mostly 102.5 to 103.5) did (FIG. 8). The 3C population density on overwintering residues was lower than that on wheat heads by the end of growing season, which was mostly above 106 target gene copies per gram sample at 44 DPI.

3C Population Level Around Harvest and During Storage

To examine the dynamics of 3C population on wheat around harvest, additional wheat head and grain samples were collected from another field (Field Two) at different time points around harvest. The 3C population levels on grains were examined again after a period of storage. The inoculated samples showed generally higher 3C detection than the non-inoculated samples for both sample types at different time points (FIG. 9). Most of the samples from inoculated plots, including the heads and the threshed grains, showed detection of higher than 103.5 and up to 106.7 target gene copies per gram. There was a trend that 3C population density on wheat heads increased from 11 days pre-harvest to 1 day pre-harvest in both inoculated and non-inoculated plots. For the samples collected from non-inoculated plots, 3C population density was significantly lower on the threshed grains of 14 days postharvest (mostly around 103.5 target gene copies per gram) than on the heads of one day pre-harvest (mostly between 104.0 and 104.5 target gene copies per gram) (P=0.037 for Tukey's test). The inoculated samples showed a trend of such difference but it wasn't significant (P=0.22 for Tukey's test). There was a trend of progressive decrease in 3C population level on threshed grains from both inoculated and noninoculated plots during storage from 14 to 64 and to 155 days postharvest (FIG. 9).

Culture-Based Quantification of 3C on Grains in Comparison to qPCR.

To investigate how well the DNA molecules detected by qPCR assay represent viable 3C cells, 3C population was quantified by colony forming units (CFU) on threshed grains at 155 days postharvest. Consistent with the qPCR results, CFU level of 3C was significantly higher on inoculated than on non-inoculated samples (FIG. 10A). Population levels lower than 1.3×10³ (103.1) units per gram grain were detected by the culture-based method (FIG. 10A) but not by qPCR (FIG. 9). There was a strong linear correlation between the Log 10 values of 3C CFU and those of target gene copy numbers given by qPCR for the inoculated samples (FIG. 10B). Population level estimated by qPCR tended to be lower than that by CFU but the magnitudes given by the two methods were similar.

Filamentous fungi were also observed on the CFU quantification plates. The dominant colonies were identified by ITS regions as Alternaria alternata and Clasdosporium clasdosporioides, which are common saprophytes. The population levels were lower than 103 CFU per gram grain for both of the dominant filamentous fungi on most of the plates and did not greatly interfere with the quantification of 3C.

Discussion

Effectiveness of the qPCR Assay.

AqPCR assay of SYBR® Green chemistry was developed to monitor 3C dispersal and persistence on field-grown wheat and residues. This assay enabled the detection of 3C with clear differential between the wheat head samples supposedly positive versus negative for 3C colonization (FIG. 5, rightmost two lanes). And it can be reasonably applied to multiple sample types, since it gave meaningful differential results within each of the sample types tested in this study.

The sensitivity of the assay is good when compared to other related qPCR assays. Previous studies using qPCR to detect fungal pathogens on wheat showed minimum detections at the magnitude of 10¹ gene copies per reaction and 10⁴ gene copies or 1 to 10³ ng genomic DNA per gram wheat tissue, which were comparable to or higher than those in the assay (12.5 gene copies per reaction and 10³ to 10⁴ gene copies or 0.01 to 0.1 ng genomic DNA per gram wheat tissue) (FIGS. 1, 3B and 4).

3C colonization of wheat heads has been previously quantified by colony forming units (CFU) (ref. Schisler 2009, 2010 Nat. FHB Forum Proc.). Applying 3C inoculum of approximately 3×10⁸ CFU/ml at the rate of about 106 CFU per square centimeter resulted in 101 to 102 CFU/g glume tissue at 16 h post inoculation and 10⁵ to 10⁶ CFU at 256 to 280 h post inoculation. 3C CFU level decreased by a magnitude of 10⁴ to 10⁵ compared to the application rate in the 16 h post inoculation in their studies. In contrast, there was only an approximately 10-fold decrease in 3C DNA density during the one day post inoculation in this study. This dramatic difference is possibly due to that most of the 3C DNA detected on 1 DPI in this study belonged to non-viable cells or was already released from lysed cells. However, 3C CFU level increased to the similar level to the application rate ten days later in their studies, which is also the case in this study. This indicates that most of the 3C DNA detected on 10 DPI belonged to viable cells.

The disclosed assay showed a certain level of inter-strain specificity within the species of Cryptococcus flavescens, where six strains other than 3C were differentiated into two genotypic groups based on amplification efficiency (FIG. 5). Even though three other strains showed similar amplification efficiency to 3C, the possibility should be minimal that native populations of other C. flavescens strains in the field interfered with the quantification of 3C population in this study. First, almost all the non-inoculated samples on 0 and 1 DPI showed no detection of targeted amplification while the inoculated samples showed clear detection (FIG. 7). If there had been a significant interference, those non-inoculated samples might have shown higher levels of detection of amplification. Second, the level of detection generally decreased as the distances from inoculated plots increase (FIG. 7). If there had been a significant interference, the levels of detection should have been generally the same or more random across the field.

3C Population Dynamics Across the Field

It is well known that microbial populations die off when they reach the death phase of growth. In some studies on the population dynamics of plant-associated bacteria using culture-based methods, decreased bacterial population on flowers or leaves of host plants later in the experimental time courses were observed. In a study using qPCR of Taqman chemistry for population quantification, the total population of three Fusarium species died off significantly on spring wheat stubble residue in the six months following summer harvest. In previous studies on 3C colonization of wheat using CFU-based quantification, slight dieoff of 3C population was observed between 184 and 256 to 280 h after wheat flowering and inoculation in the 2009 run but not in the 2010 run. In contrast, the disclosed study showed no observation of significant die-off of 3C population on the same types of samples over time, except on wheat heads from 0 to 1 DPI (FIG. 6, 8, 9). This could be due to either 3C cells able to survive over the time courses when no significant die-off was observed or 3C DNA mostly remaining intact while cells no longer viable.

The dispersal pattern of 3C across the wheat field in this study resembles the dispersal of several fungal diseases on wheat. In these situations, at the same time point, the microorganism population decreased steeply from artificially inoculated area (source) to non-inoculated area (sink) near the source, while decreased gently over farther distances across the sink, especially at early time points. In this study, there were obvious increases in 3C population at the same distances in the sink over time (FIG. 7), which is more similar to the dispersal pattern of a wheat pathogen on pure stand of susceptible cultivar than to on a mixture of susceptible and resistant cultivars. This indicates that wheat is compatible host to 3C, which is consistent with the fact that 3C is able to colonize wheat heads effectively ((Schisler, et al. In 2009 National Fusarium Head Blight Forum Proceedings, Orlando, Fla. P 80-84, 2009; Schisler D A, et al. In 2010 National Fusarium Head Blight Forum Proceedings, Milwaukee, Wis. p 98-102, 2010), And the disclosed assay is robust enough to successfully demonstrate this pattern.

Application of the qPCR Assay.

This assay can be used to conduct environmental fate and risk assessment of 3C as a biopesticide and to study the effects of common field abiotic and biotic factors of concern on 3C population dynamics in order to optimize the formulation of 3C-based biopesticides. It can also be used to investigate the association between 3C population dynamics and biocontrol efficacy of 3C against Fusarium head blight under certain microenvironments. This will facilitate the development of 3C as a biopesticide and provide guidance in the frequency and timing of 3C application. And the study on the correlation between the qPCR- and culture-based population quantification of 3C will enable better interpretation of the qPCR assay results. In addition, the target genes of this assay and other conserved genes of multiple C. flavescens strains can be sequenced to develop related qPCR assays that have different levels of specificity to serve different research purposes.

### Example 4

### Genotypic and Phenotypic Characterization of Cryptococcus flavescens, Biocontrol Agents for Fusarium Head Blight of Wheat

Materials and Methods

Strains. Cryptococcus flavescens strain OH182.9_{—}3C (3C) was obtained from the ARS Culture Collection at the NCAUR laboratory under accession number NRRL Y-50378. Twelve other C. flavescens strains were also obtained from the ARS Culture Collection under accession numbers Y-1401^{T} (=CBS 942^{T}), Y-7372, Y-7373, Y-7374, Y-7375, Y-7376, Y-7377, Y-7379, YB-328, YB-601, YB-602 and YB-744. The information on strain isolation is summarized in Table 4. All the strains were maintained as glycerol stocks at −80° C. for long term storage.

Identification of MLST loci and choice of primers. The target genes of MLST were identified from 3C draft genome (GenBank accession numbers CAUG01000001 to CAUG01000712) through blastn and blastx search against NCBI Nucleotide collection (nr/nt) database. The boundaries of exons and introns were defined through Augustus prediction and alignment to C. neoformans JEC21 (GenBank accession numbers AE107341 to AE107356). Primers used for MLST were designed using software Primer 3 (Table 5).

DNA isolation, PCR and sequencing. Genomic DNA of 3C and the 12 other C. flavescens strains were extracted from 2-day old cultures grown on ⅕ Tryptic Soy Agar using the PowerSoil™ DNA Isolation Kit (MO BIO Laboratories, Inc., California, USA) following the manufacturer's instruction. Each PCR reaction had a total volume of 25 μl, consisting of 2.5 μl genomic DNA of a C. flavescens strain, 0.2 μl solutions of each primer (100 μM), 2.5 μl mixture of four deoxynucleoside triphosphates (2 mM each, mixed at 1:1:1:1 ratio), 1.8 μl 25 mM MgCl₂, 0.3 μl GoTaq® DNA polymerase (5 U/μl), 5 μl 5× Colorless GoTaq® Flex Buffer (Promega Corporation, Wisconsin, USA) and DNA-free PCR water as the rest of the volume. Thermal cycling program of 95° C. for 7 min, then 33 cycles of 94° C. for 1 min and 72° C. for 2 min, followed by 72° C. for 8 min was run on Peltier Thermal Cycler PTC-200 (MJ Research, Inc., Massachusetts, USA). Both strands of amplification products were sequenced by the Molecular and Cellular Imaging Center at The Ohio State University (Wooster, Ohio, USA). Sequences were edited and concatenated using software Geneious 6.1.5 (created by Biomatters). The majority of processed sequences were in high quality, where greater than 95% of the bases had Phred quality scores >40. The few lower-quality bases were manually examined and base-called with enough confidence that they did not compromise the accuracy of phylogenetic analysis.

TABLE 4
C. flavescens strains used in Example 4

| NRRL | | | |
|---|---|---|---|
| Accession No. | Isolation substrate | Isolation location | Reference |
| Y-1401 (Type | Atmosphere | Japan | Saito (1922) |
| strain) | | | |
| Y-50378 | Wheat anther | Ohio or Illinois, USA | Khan et al. (2004) |
| (OH182.9_3C) | | | |
| Y-7372 | Surface of dent corn kernels of moisture 15% | Central Illinois, USA | Kurtzman (1973) |
| Y-7373 | Surface of dent corn kernels of moisture 15% | Central Illinois, USA | Kurtzman (1973) |
| Y-7374 | Surface of dent corn kernels of moisture 30% | Central Illinois, USA | Kurtzman (1973) |
| Y-7375 | Surface of dent corn kernels of moisture 12% | Central Illinois, USA | Kurtzman (1973) |
| Y-7376 | Surface of dent corn kernels of moisture 17% | Central Illinois, USA | Kurtzman (1973) |
| Y-7377 | Surface of dent corn kernels of moisture 20% | Central Illinois, USA | Kurtzman (1973) |
| Y-7379 | Surface of dent corn kernels of moisture 25% | Central Illinois, USA | Kurtzman (1973) |
| YB-328 | Soil | Hatch^{a} | NRRL catalog |
| YB-601 | Enzyme solution contaminant | R. Dimler, NRRL^{a} | NRRL catalog |
| YB-602 | Enzyme solution contaminant | R. Dimler, NRRL^{a} | NRRL catalog |
| YB-744 | Unknown | Nelson, Iowa State | NRRL catalog |
| | | University, Ames, | |
| | | Iowa^{a} | |

^{a}This information is from the “Source” field of NRRL catalog and may not reflect the actual geographic location of isolation, which could not be identified from available records.

TABLE 5
Primers and target loci for the genotypic analysis of C. flavescens strains.

| | | | Scaffold | Number of bp^{a} | | | Exon/Intron^{b} | | |
|---|---|---|---|---|---|---|---|---|---|
| Primer | | Target | accession | Whole | Amp- | Used in | Num- | Total | Refe- |
| name | Sequence (5′-3′) | locus | number | gene | lified | MLST | ber | bp^{a} | rence |
| ITS4 | TCCTCCGCTTATTGAT | 18S | CAUG01000137 | Longer | 553 | 531 | 1/2 | 247/284 | White |
| | ATGC (SEQ ID | (partial)- | | than | | | | | et al. |
| | NO: 73) | ITS1-5.8S- | | the | | | | | |
| ITS5 | GGAAGTAAAAGTCGT | ITS2- | | scaf- | | | | | 1990 |
| | AACAAGG (SEQ ID | 25S | | fold | | | | | |
| | NO: 74) | (partial) | | | | | | | |
| btb.m1-2F | GGGCGGTCTTGATTGA | β-tubulin | CAUG01000343 | 1641 | 585 | 534 | 4/3 | 379/155 | This |
| | CTTGGAACC (SEQ | | | | | | | | study |
| | ID NO: 75) | | | | | | | | |
| btb.m2R | TCGTTGTCGATACAGA | | | | | | | | |
| | AGGTCGCGT (SEQ | | | | | | | | |
| | ID NO: 76) | | | | | | | | |
| CS24.m2F | ACAGGCTTGGAGGGA | Chitin | CAUG01000683 | 3840 | 356 | 332 | 2/1 | 229/103 | |
| | GAAGATCACG (SEQ | synthase 1 | | | | | | | |
| | ID NO: 77) | | | | | | | | |
| CS24.m2R | CGAACGAGGTAGAAT | | | | | | | | |
| | CAGGCTTCGC (SEQ | | | | | | | | |
| | ID NO: 78) | | | | | | | | |
| EF1.m2F | CGTGGTACAAGGGATG | Elongation | CAUG01000040 | 2012 | 658 | 628 | 3/2 | 450/178 | |
| | GACCAAGGA (SEQ | factor 1 | | | | | | | |
| | ID NO: 79) | | | | | | | | |
| EF1.m2R | CAATGTGGGCAGTGTG | | | | | | | | |
| | GCAGTCAAG (SEQ | | | | | | | | |
| | ID NO: 80) | | | | | | | | |
| h31.m1F | ATGTACGCCGCTACCC | Heat shock | CAUG01000237 | 2261 | 719 | 698 | 5/4 | 508/190 | |
| | GATCTCTTC (SEQ | protein | | | | | | | |
| | ID NO: 81) | 70 kDa | | | | | | | |
| h31.m1R | CGACAAAGGCACCAA | | | | | | | | |
| | TTTGCGATGG (SEQ | | | | | | | | |
| | ID NO: 82) | | | | | | | | |
| CS22.m2F | AAGAAGGGAGTGAGG | cs22 (chitin | CAUG01000018 | 6412^{c} | 91, | 83, | 1/0^{c} | 83/0^{c} | |
| | AAGCAGCTCG (SEQ | synthase | | | 771^{d} | 724^{d} | | | |
| | ID NO: 83) | 5 plus | | | | | | | |
| CS22.m2R | ACTTGTTCGCCTCTTG | downstream | | | | | | | |
| | GTAGACGCT (SEQ | anonymous | | | | | | | |
| | ID NO: 84) | region) | | | | | | | |

Phylogenetic analysis. Processed sequences were aligned and sequence identities were calculated using ClustalW2. Phylogenetic trees were generated using Maximum Likelihood method with General Time Reversible model in MEGA 5. Bootstrap test of 1,000 replicates were conducted to determine the reliability of consensus trees. To evaluate the effect of using different tree building methods, trees were also generated for all the relevant alignments using Neighbor Joining method in MEGA 5 and Bayesian inference in MrBayes v3.2.0, resulting in equivalent tree topologies to those by Maximum Likelihood method.

Biochemical assay. Profiles of carbon source utilization and chemical sensitivity were generated using Biolog GEN III Microplates (BIOLOG, Inc., California, USA). Two-day-old C. flavescens colonies grown on ⅕ Tryptic Soy Agar were suspended in 120 μl of sterile double distilled water. Optical density of cell suspensions was measured at 595 nm using an ELx800 Universal Microplate Reader (BIO-TEK Instrument, Inc., Vermont, USA). Forty to 100 μl of cell suspension in water was added to 10 ml inoculation fluid IF-A (BIOLOG, Inc., California, USA) and mixed well by vortexing. One hundred μl of cells suspended in IF-A (3×10⁵ to 4×10⁵ cells) was added to each well of a Biolog Gen III Microplate. Assay plates were incubated at room temperature (23 to 25° C.) in the dark. Color change of assay wells, representing C. flavescens growth and cell respiration, was measured as OD₅₉₅ by the ELx800 Universal Microplate Reader following incubation. Two experiments were set up. Twelve strains (3C, Y-7372, Y-7373, Y-7374, Y-7375, Y-7376, Y-7377, Y-7379, YB-328, YB-601, YB-602 and YB-744) were used in Experiment One, and four strains (Y-1401T, 3C, Y-7372 and YB-601) were used in Experiment Two.

Statistical analyses were conducted on OD₅₉₅ data in Minitab 15 (Minitab Inc., Pennsylvania, USA). Cluster Variables analysis was performed on the data from all the assay wells as a whole using Correlation Coefficient Distance and a variety of Linkage methods including Average, Complete, McQuitty, Single and Ward. To avoid mistaking run-to-run variation for real phenotypic difference among strains, the minimum pair-wise similarity percentage between the two independent runs of the same strain was used as the lower similarity threshold among the strains forming one distinct group. Analysis of Variance (ANOVA) was performed on the data of individual assay wells to compare distinct groups using General Linear Model with an α value of 0.01, 0.03 or 0.1 for defining significant difference in F test. In Experiment One, the data from 7 days of incubation in Run I and 10 days in Run II (referred as short incubation) were combined and those from 12 days in Run I and 15 days in Run II (referred as long incubation) combined for ANOVA. In Experiment Two, the data from the same days of incubation in both runs were combined for ANOVA.

Biocontrol assays. Macroconidia of F. graminearum isolate Z-3639 were produced on clarified V8 juice agar (CV8 agar) under a regime of 12 h/day fluorescent light for seven days at 25° C. Colonized plates were flooded with weak PO₄ buffer to obtain conidial suspensions of the pathogen. Biomass of all C. flavescens strains except Y-7374, Y-7375, Y-7376, Y-7377, Y-7379 was initiated on plates of one-fifth strength Tryptic soy broth agar (TSBA/5). After 24 h incubation at 28° C., cells were used to inoculate 50 ml of semi-defined complete liquid medium (SDCL) in 250-ml Erlenmeyer flasks (OD=0.10, A620). Flasks were incubated in a shaker incubator (250 rpm, 25° C., 24 h) to produce cells in log growth to inoculate cultures (50 ml SDCL in 250-ml flask, OD=0.10, A620) that were then used as inoculum cultures after a 48 h incubation.

Each experiment to determine strain efficacy was conducted in a climate-controlled greenhouse with temperatures that ranged from 17 to 20° C. at night and 25 to 28° C. during the day. Natural sunlight was supplemented with high-pressure sodium lights for 14 h/day. Each experimental unit consisted of two plants of hard red spring wheat cultivar Norm grown in a 19-cm-diameter plastic pot. Each pot contained air-steam pasteurized (60° C. for 30 min) potting mix (Terra-lite Redi-earth mix, W. R. Grace, Cambridge, Mass.) and plants were grown in a growth chamber (25° C., 14-h photoperiod, 600 u′ mol/[m²/s]) for 7-8 weeks prior to transfer to greenhouse benches. Pots were fertilized after 1 week and weekly thereafter with 50 ml of a solution containing 1.25 g/liter Peters 20-20-20 (Grace-Sierra Horticultural Products, Milpitas, Calif.) and 0.079 g/liter iron chelate (Sprint 330, Becker Underwood, Inc., Ames, Iowa). Approximately one week after transferring plants to the greenhouse, wheat heads were inoculated at anthesis by spraying with 25% freshly harvested C. flavescens cells (˜5×10⁷ CFU/ml) in PO₄ buffer and a final concentration of 0.036% Tween 80 (Sigma Chemical Co., St. Louis, Mo.). For each experiment, 50 mL of inoculum of each strain was used to inoculate six plants representing a total of 12 to 15 heads. Heads were then challenged immediately by spraying 12 mL of a conidial suspension of F. graminearum (3×10⁴ conidia/ml) in PO₄ buffer with 0.036% Tween 80. Wheat heads inoculated first with the PO₄ buffer/tween 80 solution and then only with F. graminearum conidia in PO₄ buffer/tween 80 served as a pathogen only control. Uninoculated pots of wheat were used to ensure pathogen inoculum did not spread between treatments. Inoculated plants were misted lightly with distilled water and incubated in a plastic humidity chamber at 17 to 20° C. at night and 25 to 28° C. during the day for 3 days before being transferred to greenhouse benches. Treatments were arranged in a completely randomized design. Fusarium head blight severity was visually estimated using a 0 to 100% scale at 10 and 16 days after inoculation. All greenhouse experiments were conducted five times. Data were analyzed using the Kruskal-Wallis test using Minitab v.15.

Accession numbers. The sequences used in MLST analysis were deposited in the GenBank/EMBL/DDBJ databases under accession numbers KC679238 to KC679297 and KF171106 to KF171122.

Results

Evidence for Two Distinct Genotypes of C. flavescens.

Six loci-rDNA-ITS, the partial sequences of four protein-encoding genes and cs22 were sequenced and aligned to infer phylogenetic relationship among the C. flavescens strains (Table 5). These loci were probably unlinked since they were from six different scaffolds in the draft genome of 3C and their homologs were located on six different chromosomes of C. neoformans JEC21 (GenBank accession numbers AE107341 to AE107356), respectively. The rDNA-ITS sequences of all 13 strains were 100% identical. The other five loci revealed divergence within the species (thus termed variable loci), resulting in two clades (genotypes A and B) supported by a bootstrap value of 100 (FIG. 11). For the four protein-encoding genes, percentage of identical sites in alignment ranged from 95.8% to 100% within each genotype and 89.5% to 95.1% between the two genotypes, where the chitin synthase 1 and β-tubulin loci showed the highest and lowest levels of divergence, respectively (Table 6). Locus cs22 is the 3′ end of a homolog to the terminal exon of a chitin synthase 5 gene in C. neoformans JEC21 (83 bp) plus its downstream anonymous region (724 bp) identified from the draft genome of 3C. The anonymous region is possibly intergenic because no reasonable coding sequences were predicted within it by Augustus and that neither discontiguous megablast or blastx search against the NCBI Nucleotide Collection (nr/nt) returned any hit for it. The sequence difference between the two genotypes at cs22 was much lower than those for the four protein-encoding genes (Table 6), partially due to a 36-bp insertion (in genotype A) or deletion (in genotype B) in the anonymous portion. The sequence difference within each genotype at cs22 was medium to high instead of low as well compared to the four protein-encoding genes (Table 6).

The genotype split was consistent across all the five variable loci (FIG. 14). There were two to six strains of each genotype showing 100% pairwise identity to one another at each of the five loci. In addition, all the corresponding sequences were 100% identical within each of the following groups: (i) 3C, Y-1401^{T}, and Y-7373; (ii) YB-601 and YB-602; (iii) Y-7372, Y-7374, Y-7375 and (iv) YB-7379 and YB-744. The minimum pairwise identities for each locus were the same as or very similar to the percentage of identical sites in alignment. Y-7377 was divergent from the rest of the genotype-A strains by pairwise identities of 95.8%, 99.0% and 99.3% for three loci-chitin synthase 1, heat shock protein 70 kDa and cs22 (FIGS. 14B, 14D, and 14E; Table 6).

Taxometric Analysis of Biochemical Properties of C. flavescens Strains.

While sharing many similarities in response to the Biolog GEN III substrates, 12 C. flavescens strains (not including Y-1401^{T}) could be split into three measurably different groups, depending on incubation time (FIG. 12). Common to all isolates was the good assimilation of 31 out of the 71 assayed carbonic compounds (Table 7, Row 1) and poor assimilation of 22 others (Table 7, Row 2). Additionally, all the 12 strains were resistant to 18 out of 23 types of chemical stress (Table 7, Row 1) and sensitive to Tetrazolium Violet. All the 7 genotype-B strains formed a distinct group from the genotype-A 5 strains after both short and long periods of incubation (FIGS. 2A and 2B, respectively). At the end of the long incubation period, the basis of this grouping was found to come from difference in utilization of 11 carbon sources and sensitivity to 3 compounds (Table 7, Row 3)

TABLE 6
DNA sequence identities among C. flavescens strains.

| | % identical sites^{a} among . . . | | |
|---|---|---|---|
| | | | all the |
| Target locus | Genotype A^{b} | Genotype B^{b} | strains |
| 18S-ITS1-5.8S-ITS2-25S | 100 | 100 | 100 |
| β-tubulin | 100 | 99.8 | 95.1 |
| Chitin synthase 1 | 95.8 | 98.8 | 89.5 |
| Elongation factor 1 | 99.7 | 99.7 | 94.1 |
| Heat shock protein 70 kDa | 99.0 | 99.1 | 93.7 |
| cs22 (chitin synthase 5 plus | 99.3 | 100 | 79.4 |
| downstream anonymous region) | | | |
| Concatenation of the 5 regions | 99.0 | 99.6 | 89.8 |
| above (excluding rDNA-ITS) | | | |

^{a}The percentage of columns in the alignment for which all the sequences were identical.
^{b}The strains classified as genotype A are 3C, Y-1401^{T}, Y-7373, Y-7377, YB-601 and YB-602; those classified as genotype B are Y-7372, Y-7374, Y-7375, Y-7376, Y-7379, YB-328 and YB-744.

Genotype-A strains showed different subgrouping patterns depending on incubation time (FIG. 12A vs. FIG. 12B). YB-601 and YB-602 were quite distinct from all the other 10 strains after short incubation (FIG. 12A) while clustered together with the rest of genotype-A strains after long incubation (FIG. 12B). One of the contributors to this discrepancy is probably the unique utilization patterns of several carbon sources by YB-601 and YB-602, such as Tween 40, L-alanine, D-trehalose and L-malic acid. OD₅₉₅ of these four assay wells were significantly lower for YB-601 and YB-602 than for the rest of genotype-A strains (P<0.01 for F test) after short incubation while not significantly different among the two sub-groups of genotype A (P>0.1 for F test) after long incubation. This indicated that YB-601 and YB-602 assimilated these carbon sources more slowly, resulting in a lagged growth compared to the other genotype-A strains. This phenotypic difference is consistent with the divergence of YB-601 and YB-602 from the three genotype-A strains in the phylogeny of elongation factor 1 (FIG. 14C). After long incubation, Y-7373 and Y-7377 formed a phenotypically distinct group from 3C, YB-601 and YB-602 by a similarity slightly lower than the threshold (FIG. 12B). This is partially consistent with the divergence of Y-7377 from the other three strains at the loci of chitin synthase 1, heat shock protein 70 kDa and cs22 (FIGS. 14B, 14D, and 14E). The use of other Linkage methods (Average, McQuitty, Single and Complete) than Ward in Cluster analysis resulted in different topologies and similarity magnitudes. However, the aforementioned clustering patterns were always demonstrated regardless of Linkage method.

TABLE 7
Phenotypic characteristics of C. flavescens strains displayed in the Biolog GenII assays.

| Characteristic | Carbon source | Chemical stress |
|---|---|---|
| Good growth of all 12 | D-cellobiose, stachyose, D-trehalose, D-turanose, | 1% sodium lactate, 1% NaCl, |
| strains (OD₅₉₅ > 0.6), | α-D-lactose, β-methyl-D-glucoside, γ-amino- | potassium tellurite, pH 5, pH |
| sorted by mean OD₅₉₅ | butryric acid, D-mannitol, D-melibiose, D- | 6, aztreonam, nalidixic acid, |
| among strains in | raffinose, D-maltose, D-galactose, D-fructose, L- | lincomycin, rifamycin SV, |
| descending order^{a} | pyroglutamic acid, sucrose, L-glutamic acid, | vancomycin, minocycline, |
| | gentiobiose, L-rhamnose, L-malic acid, D-arabitol, | sodium butyrate, fusidic acid, |
| | L-arginine, D-gluconic acid, L-aspartic acid, L- | D-serine, 4% NaCl, sodium |
| | galactonic acid lactone, L-alanine, D-mannose, | bromate, troleandomycin |
| | acetic acid, D-galacturonic acid, α-D-glucose, | |
| | glycyl-L-proline, L-serine | |
| Poor to no growth of all | α-Hydroxy-butyric acid, p-hydroxy-phenylacetic | Tetrazolium violet |
| 12 strains (OD₅₉₅ < | acid, quinic acid, propionic acid, β-hydroxy-D,L- | |
| 0.6), sorted by mean | butyric acid, formic acid, inosine, n-acetyl | |
| OD₅₉₅ among strains in | neuraminic acid, gelatin, D-serine, α-keto-butyric | |
| ascending order^{a} | acid, D-glucose-6-PO₄, L-histidine, N-acetyl-D- | |
| | galactosamine, D-lactic acid methyl ester, N- | |
| | acetyl-β-D-mannosamine, pectin, N-acetyl-D- | |
| | glucosamine, D-fructose-6-PO₄, citric acid, | |
| | acetoacetic acid | |
| Significant difference | Propionic acid, L-histidine, acetoacetic acid, | Lithium chloride, Niaproof 4, |
| between genotypes A | glucuronamide, D-aspartic acid, D-malic acid, L- | tetrazolium blue |
| and B (P < 0.01 for F | lactic acid, 3-methyl glucose, p-hydroxy- | |
| test), sorted by P-value | phenylacetic acid, pectin, glycyl-L-proline | |
| in ascending order^{a} | | |
| Significantly poorer | D-raffinose, α-D-glucose, D-melibiose, D-fructose, | D-serine, 1% NaCl, pH 5, |
| growth of Y-1401^{T} than | D-galactose, α-D-lactose, D-cellobiose, β-methyl- | sodium bromate, potassium |
| three other strains (P < | D-glucoside, dextrin, D-mannose, D-galacturonic | tellurite, 1% sodium lactate, |
| 0.03 for F test), sorted | acid, bromo-succinic Acid, L-aspartic acid, D- | minocycline, fusidic acid, |
| by P-value in ascending | glucuronic acid, sucrose, mucic acid, L-alanine, | aztreonam, guanidine HCl, pH |
| order^{b} | Tween 40, L-rhamnose, glycerol, D-maltose, L- | 6, nalidixic acid, sodium |
| | serine, D-fucose, D-salicin, L-arginine | butyrate, troleandomycin, |
| | | vancomycin |
| Consistent with | N-acetyl-D-glucosamine (−), D-cellobiose (+), D- | Not applicable |
| previous studies^{c} | galactose (+) (expect Y-1401^{T}), D-galacturonic | |
| | acid (+), D-gluconic acid (+), α-D-glucose (+), D- | |
| | glucuronic acid (+), sucrose (+), D-raffinose (+), | |
| | D-melibiose (+), L-lactic acid (v), α-D-lactose (+), | |
| | D-maltose (+), β-methyl-D-glucoside (+), myo- | |
| | inocitol (+), D-salicin (+) | |

^{a}Information in these rows is based on the Experiment One data combined from 12 days of incubation in Run I and 15 days of incubation in Run II. Y-1401^{T} was not included.
^{b}Information in this row is based on the Experiment Two data combined from 19 days of incubation in both Run I and Run II.
The results from previous studies were indicated as “+” for positive growth, “−” for negative and “v” for variable in the parentheses following each chemical. In our study, OD₅₉₅ was >0.6 for “+”, <0.4 for “−” and a mixture of >0.6 and <0.4 for “v” by 19 days of incubation in Experiment Two Run II for Y-1401^{T} and by15 days of incubation in Experiment One Run II for the other 12 strains. OD₅₉₅ of Y-1401^{T} for D-galactose utilization assay was 0.472.

In subsequent analyses, Type strain Y-1401^{T} showed distinct biochemical properties from 3C, YB-601 (both genotype A) and Y-7372 (genotype B) regardless of incubation time (data not shown). This distinction was probably due to the poorer growth of Y-1401^{T} than the other three strains, with statistical significance in 27 out of 71 carbon source utilization assays and 16 out of 23 chemical sensitivity assays (Table 7, Row 4). The only exception was that Y-1401^{T} utilized citric acid slightly better than the other strains by the end of 19 days' incubation (P=0.086 for F test). For each individual run, 3C and Y-7372 formed a different cluster from YB-601 after 5 days' incubation, while 3C and YB-601 formed a different cluster from Y-7372 after 19 days' incubation. These patterns were consistent with the two runs (FIGS. 12A and 12B). However, the run-to-run variation in subsequent assays using the type strain was so high that such patterns were not observed when the data from both runs were viewed together.

Biocontrol Efficacy of the Two C. flavescens Genotypes in Suppressing Fusarium Head Blight (FHB).

Representative strains from the two genotypes were assessed for FHB suppression on wheat in five separate greenhouse bioassays. Reductions in FHB disease severity by pre-inoculation with C. flavescens isolates were observed in four of five independent trials. In two of those four trials, the observed reduction in disease was statistically significant (P<0.10, Kruskal-Wallis test) after 16 days of incubation. In total, the genotype-A isolates displayed greater biocontrol activity than the genotype-B isolates in three of five independent trials, but given the high degree of variation of disease scores that are typical of such trials, the individual run results were not statistically significant. However, considering all of the tested strains, significant reductions in disease severity due to inoculation with C. flavescens were observed at both 10 and 16 days post-inoculation (P=0.11 and 0.03, respectively, Kruskal-Wallis test; FIG. 13). Furthermore, as a subgroup, genotype A isolates reduced disease severity relative to the negative control after 16 days of incubation (P<0.10 Kruskal-Wallis test) in the greenhouse trials. But, while genotype B isolates also appeared to reduce disease severity on average, the trend shown in FIG. 13 was not statistically significant for that subgroup of C. flavescens. As a group, genotype A reduced average FHB disease severity at 10 days post inoculation to 16% (versus 23% observed for the negative control) and at 16 days to 46% (versus 64% for the negative control). Similarly, but to a lesser extent, genotype B reduced average disease severity to 17% and 51% at 10 and 16 days, respectively. These data demonstrate that as a whole, isolates of C. flavescens have the capacity to control Fusarium head blight, and of the two groups identified in this work, genotype A is more effective at controlling FHB than genotype B.

Discussion

This example provides the first detailed characterization of C. flavescens diversity, and demonstrates the occurrence of genotypically and phenotypically distinct subgroups within the large majority of publicly available isolates of the species. Previously, the only available sequences of C. flavescens were the ribosomal DNAs and internal transcribed spacers of type strain Y-1401^{T} (=CBS 942^{T}) and several other strains. In contrast to the intra-species diversity at the rDNA-ITS loci of human-pathogenic C. neoformans and biocontrol fungus Trichoderma harzianum, all of C. flavescens rDNA-ITS sequences obtained in this study were 100% identical to the published sequence of Y-1401^{T} (GenBank accession number AB035046). Since a strain with 99.0% identity to Y-1401^{T} in ITS region has been identified as C. flavescens, all the other 12 strains in this study would typically be classified as C. flavescens as well based on ITS genotype alone. However, this group was capable of being split into two distinct genotypes based on MLST (FIG. 11), with greater than 10% sequence divergence in three of the five amplified sequences used (Table 6). Phenotypically, all the genotype-A strains formed a distinct group from genotype-B strains regardless of run-to-run variation (FIG. 12). This demonstrates that genotype is indicative of biochemical properties for C. flavescens. And, most relevant to plant pathologists, both genotypes of C. flavescens showed biocontrol efficacy against FHB (FIG. 13), which indicates that the species as a whole has significant biocontrol potential. Additionally, genotype A (for which strain 3C is the type strain) performed slightly better than genotype B when combining data from five independent greenhouse experiments.

The genotype split shown by the five variable MLST loci was consistent with the results observed during the development of a quantitative PCR (qPCR) assay for C. flavescens (see Example 5). In Example 5, a 150-bp region within the same heat shock protein 70 kDa locus used in MLST of this study was targeted. The qPCR assay differentiated the tested C. flavescens strains into two groups, 3C like and non-3C like, based on difference in threshold cycles that resulted from sequence difference in primer binding regions. The 3C like strains actually belong to genotype A and non-3C like to genotype B. Within genotype A, the 100% identity of Y-1401^{T} to 3C and Y-7373 at the five variable MLST loci indicates that Y-1401^{T}, though isolated in Japan many decades earlier, is closely related to the two strains isolated more recently in the USA. Thus, the species appears to be genetically stable on a decadal time scale.

The utilization results of carbon sources were consistent most of the time between previous studies and this study (Table 7, Row 5). The only minor inconsistency was glycerol. The results were previously negative for Y-1401^{T} or variable. However, it was positive or near positive for utilization by all the 13 strains in the present study (OD₅₉₅=0.589 for Y-1401^{T} and OD₅₉₅>0.7 for the other 12 strains after longest incubation). Though Y-1401^{T} showed minimal genetic divergence from most of the genotype-A strains, its Biolog assay results were highly distinct due to the generally poorer growth. It is unknown whether this pattern resulted from geographic/habitat difference or sub-culturing/storage of strains over the years. Carbon source utilization has been previously related to recovery of biocontrol agents against Fusarium head blight (FHB), where utilization of tartaric acid by microorganisms was positively biased towards the ability to reduce FHB damages. Tartaric acid can be utilized by OH182.9 (progenitor of 3C) but not included in the Biolog assays used in this study.

Several strains were previously designated as two mating types based on a mating test where mating responses included formation of conjugation tubes and hypha growth from fused tubes in mixed culture of strain pairs. The strains of the opposite mating types are able to mate (in the lab) while those of the same mating type are not. In that study, Y-7372, Y-7374, Y-7375, Y-7376 and Y-7379 were designated as mating type α; while Y-7373 and Y-7377 as mating type a. All the five α-strains were shown as genotype B while both a-strains as genotype A by MLST in the present study. The genetic divergence between the two mating types indicated minimal sexual recombination among the studied strains in the environment. The fact that the strains of the same MLST genotype belonged to the same mating type in this study is consistent with a previous MLST study on C. neoformans var. grubii, where more than 40 MLST types and only two mating types were identified. Thus, within a Cryptococcus species, the strains of different mating types tend to be of different genotypes at other conserved loci.

Genetic markers have been used to assist discovery of novel biocontrol isolates and to study the geographic distribution of biocontrol agents. The MLST markers developed in this study may be used to identify new and novel C. flavescens biocontrol agents. MLST analysis can also be performed on additional C. flavescens strains when they are recovered from wheat fields at various geographic locations to comprehensively characterize the natural range and diversity of this species. Heterosis, where hybrid progeny shows superior biological quality over parents, has been utilized in plant breeding to produce hybrid cultivars with improved performance such as higher yield and disease resistance. Crossing C. flavescens isolates of compatible mating types can be conducted to investigate whether hybrid progeny shows increased biocontrol efficacy against FHB. The carbon source utilization and chemical resistance patterns shown in Biolog plates may also be used to develop C. flavescens-specific media aiding the selective recovery of C. flavescens as potential biocontrol agents.

### Example 5

### A Quantitative PCR Assay for Monitoring the Field Dispersal and Persistence of Cryptococcus flavescens, a Biocontrol Yeast Applied to Wheat

Materials and Methods

Strains. Cryptococcus flavescens strain OH182.9_{—}3C (3C) was obtained from the ARS Culture Collection at the NCAUR laboratory under accession number NRRL Y-50378. Six other C. flavescens strains were also obtained from NRRL under accession numbers Y-7372, Y-7373, YB-328, YB-601, YB-602 and YB-744. All the strains were maintained as glycerol stocks at −80° C. for long term storage.

Field establishment and 3C application. The experiments of this study were performed in two fields planted with soft red winter wheat (Triticum aestivum L.) in the Snyder Farm at the northern end of Wooster, Ohio. The first field, subsequently noted as Field One, was planted with Cultivar Hopewell on Oct. 11, 2010. The layout of Field One is shown in FIG. 5. The second field, subsequently noted as Field Two, was planted with Cultivar Freedom on Oct. 6, 2011. Wheat in Field Two was planted in a rectangle area of 85 m×16 m containing 14×7 plots of 4.6 m×1.7 m per plot with spaces of 1.5 m along the width and 0.6 m along the length. Corn kernels colonized by Fusarium graminearum were distributed evenly throughout Field Two approximately three weeks prior to anthesis. The data shown in FIGS. 7,15, and 16 were collected from Field One, and data shown in FIG. 17 were collected from Field Two.

Inoculum production. Log-phase cells of 3C produced in SDCL medium served as a 5% seed inoculum for the production in B Braun D-100 fermentor charged with 80 liters of SDCL medium. The fermentor was operated at 25° C., 20 liter/min aeration and 200 revolution/min with agitation provided by twin Rushton impellers. Eighteen hours after inoculation, fermentor temperature was reduced to 15° C. to cold shock the cells for 24 h prior to harvest. The cells were harvested through concentration into a paste using a Sharples 12-V tubular bowl centrifuge. The cell paste was then suspended in weak PO₄ buffer at approximately 2×10⁹ CFU/ml and frozen at −20° C. as stock. The field inoculum was prepared right before application by mixing 1.5 liter of stock (pre-thawed at 10° C. overnight) with 1.5 liter sterile double distilled water and 10.8 ml 10% Tween 80. During anthesis of the wheat (Feekes Stage 10.5, on May 29, 2011), three areas of 6.1 m by 6.1 m (Plots i_{—}1, i_{—}2, and i_{—}3 of FIG. 5) were sprayed with the 3C inoculum as a mist of approximately 1.5×10⁹ cells/ml at the rate of 2×10⁶ to 6×10⁶ cells per square centimeter.

For producing 3C inoculum for Field Two, 500-ml flasks containing 200 ml SDCL medium inoculated with 3C (starting OD of 0.10 at A₆₂₀) were incubated at 25° C. at 250 revolution/min for 24 h. These cultures were then used to seed 3-liter Fernbach flasks containing 1.25 liter of SDCL medium to an OD of 0.10. Cultures were incubated for 48 h at 25° C. and 250 revolution/min. Colonized broth of 2.3×10⁸ CFU/ml was then transferred into sterile containers, transported to the field on ice and applied at the rate of 4.4×10⁵ CFU per square centimeter in the field within 24 h. The application was performed during anthesis of the wheat (Feekes Stage 10.5, on May 29, 2012). Sampling for this study was performed in 12 plots, 6 of which were sprayed with 3C only and another 6 of which were not sprayed with any microorganism. The plot locations of different treatments were randomized across the field.

Sample collection. Wheat head samples were collected by clipping at the stalk 1 to 2 cm below the head and sealing each head in an individual sample bag. During sample collection, each head was only touched with the inside of the bag used to collect it and the clipping scissors were sterilized with 75% aqueous ethanol between clips to prevent cross-contamination. Heads were collected from Field One within 1 h after the spray application of 3C (0 day post inoculation) as well as 24 h (1 day), 10, 26 and 44 days post inoculation in the summer of 2011 (see FIG. 5 for details on the locations of sampling). Heads were also collected from Field Two at 11 days and 1 day prior to harvest on Jun. 26, 2012, where one head was collected from each of the 12 plots at each time point.

Postharvest residue samples were collected from the three inoculated areas of Field One in late January and late March of 2012. Seven different locations were sampled within a circle of about 1-m radius around the center of each inoculated area. At each time point, two types of samples were collected from each location: a mixture of soil, living weeds and brownish rotting plant materials was collected as one composite sample, and non-rotting wheat stalks were collected as the other sample.

The grains were harvested from each plot as a whole for the 12 plots of Field Two on Jun. 26, 2012. The total grains of each plot were stored in one paper bag as a layer thinner than 6 cm at dark at room temperature. One subsample of approximately 10 g was collected from the total grains of each plot immediately prior to grinding up for DNA extraction at 14, 64 and 155 days postharvest, respectively.

Sample handling, DNA extraction and template preparation. Genomic DNA of 3C and the six other C. flavescens strains was extracted from 2-day old cultures grown on ⅕ Tryptic Soy Agar using PowerSoil™ DNA Isolation Kit (MO BIO Laboratories, Inc., California, USA) following the manufacturer's instruction. These original DNA extracts were diluted by more than 20-fold to certain concentrations based on the measurements on NanoDrop ND-1000 (Thermo Scientific, Delaware, USA) to prepare the templates for qPCR.

The wheat head and stalk samples were returned to the lab within 1 hr of sampling and stored at −80° C. The wheat (including grain) samples were ground up in liquid nitrogen immediately prior to DNA extraction. The composite residue samples were stored at 10° C. after returned to the lab and then DNA extraction was done within 24 h directly from the samples without grinding in liquid nitrogen. Total DNA was extracted from approximately 0.3 gram of each sample using PowerSoil™ DNA Isolation Kit following the manufacturer's instruction, yielding 100 μl DNA extract per sample. Since the DNA concentrations of the wheat head and grain samples were so high (due to the abundant wheat DNA from fresh tissue) as to interfere with the interpretation of qPCR data, 1:20 dilutions and 1:10 dilutions of the original DNA extracts were used as templates for those samples from Field One and Two, respectively. Non-diluted DNA extracts were used as the templates of postharvest residue samples from Field One.

Quantitative PCR (qPCR). qPCR primers were designed using Primer3 software based on the draft genomic sequences of 3C (EMBL accession number CAUG01000001 to CAUG01000712). qPCR test for specificity was performed on 14 pairs of primers targeting 7 putative conserved regions, using the 1 ng/μl genomic DNA of 3C and the other 6 C. flavescens strains as template. The 7 regions were putatively ITS1-5.8S-ITS2 and partial regions of the following protein encoding genes: (3-tubulin, chitin synthase 5, elongation factor 1 and three 70 kDa heat shock proteins. Among the 14 pairs of primers, Pair h31.2 showed the greatest difference in Threshold Cycle values between 3C and any of Y-7372, YB-328 and YB-744, and was thus chosen for use in the qPCR assay described as follows. Each qPCR reaction had a total volume of 25 μl, consisting of 2.5 μl template DNA, 0.4 pmol/μl of each primer (forward primer h31.2_F: 5′-CGTCAGCGTGTTGCCACTTCGT-3′ (SEQ ID NO: 67); reverse primer h31.2R: 5′-GCTGCTGTCTTGCGGTCGCTTA-3′(SEQ ID NO: 68)), 12.5 μl iQ™ SYBR® Green Supermix (Bio-Rad Laboratories, Inc., California, USA) and DNA-free PCR water as the rest of the volume. DNA-free PCR water was also used as the template of negative controls. The thermal cycling program was run on the iCycler and the fluorescence data was collected by the iQ™5 detection system (Bio-Rad Laboratories, Inc.). Amplification was two-step with 3 min at 95° C. followed by 40 cycles of 15 s at 95° C. and 1 min at 72° C. Then the melting curve was run from 55° C. to 95° C. with an increment of 0.5° C. and a dwelling time of 10 s at each temperature. The fluorescence data of qPCR was analyzed using the iQ™5 Optical System Software (Bio-Rad Laboratories, Inc.).

Data processing and analysis. The standard series of 1, 1×10⁻¹, 1×10⁻² . . . 1×10⁻⁷ ng/μl 3C genomic DNA in template were run in triplicate to obtain the regression equation Y=5.792−0.2984X (R²=0.991). This equation was used to calculate from the field samples' Threshold Cycle values (X) their log starting quantity values (Y) as equivalences to 3C genomic DNA concentration in template. The unit of starting quantity was converted from “ng/μl 3C genomic DNA in template” to “target gene copy number per gram sample”. This unit conversion was based on the following two assumptions: (i) there was one copy of target gene per genome, supported by BLAST search of this gene and the h31.2 primers against the draft genome of 3C and by the sequencing coverage of the respective scaffold relative to the coverage of the whole genome (data not shown); (ii) the genome size of 3C was about 0.02 pg per haploid genome (estimated based on the genome sizes of Cryptococcus gattii and C. neoformans) (Fungal Genome Size Database). Accordingly, 1 ng 3C genomic DNA was considered equal to 5×10⁴ copies of target gene and, reciprocally, one copy of target gene equal to 2×10⁻⁵ ng 3C genomic DNA. In this study, each reaction started with 2.5 μl template, which belonged to a total of 100 μl DNA extract resulted from 0.3 gram sample. Therefore, 1 ng/μl 3C genomic DNA was considered equal to 3.3×10⁸ (10^{8.5}) or 1.7×10⁸ (10^{8.2}) copies of target gene per gram sample for the wheat head or grain samples (1:20 or 1:10 dilutions of original DNA extracts as templates, respectively) and 1.7×10⁷ (10^{7.2}) copies per gram sample for postharvest residue samples from Field One (non-diluted DNA extracts as templates).

The theoretical limit of detection for any given PCR reaction was considered to be one copy of target gene per reaction. Given the two aforementioned assumptions for unit conversion and that each reaction started with 2.5 μl template in this study, the theoretical detection limit of this study was 2×10⁻⁵ ng 3C genomic DNA per 2.5 μl, equal to 8×10⁻⁶ ng/μl 3C genomic DNA in template. For the field samples, of which a total of 100 μl DNA extract resulted from 0.3 gram sample, the theoretical detection limits were 2.7×10³ (10^{3.4}) or 1.3×10³ (10^{3.1}) copies of target gene per gram sample for the wheat head or grain samples (1:20 or 1:10 dilutions of original DNA extracts as templates, respectively) and 1.3×10² (10^{2.1}) copies per gram sample for the postharvest residue samples from Field One.

In the melting curve analysis for qPCR, a single peak of 87.0 or 87.5° C. was the only peak reported by the iQ™5 detection system for the concentrated templates in the standard series (1 through 10⁻⁴ ng/μl 3C genomic DNA). Therefore for field samples, only those where a single peak of 87.0 or 87.5° C. was the only reported peak were considered as positive for detection of 3C. The samples showing no peak of 87.0 or 87.5° C. were considered as no detection of 3C and their Ct values were considered as 41 for statistical analysis. The samples showing a peak of 87.0 or 87.5° C. as well as other peaks were trimmed out of further analysis since they showed detection of 3C but the quantification will not be precise enough due to the interference of non-specific peaks. A few statistical outliers were also trimmed out. For each experiment, the trimmed-out data points never exceeded 7% of the total data points, unless specified. Analysis of variance using General Linear Model where α=0.05 was performed on the ranked forms of Ct values using the software Minitab 16 (Minitab Inc., Pennsylvania, USA).

Results

Sensitivity and Specificity of the Assay.

The primer pair h31.2 used in the qPCR assay were designed to amplify part of a putative heat shock protein 70 kDa (hsp70) gene identified from the draft genomic sequence of 3C. Primer h31.2F binds to a putative intron region and primer h31.2R binds to a putative exon region (FIG. 18). The target region was 150 bp and includes one putative exon-intron conjunction. The amplification efficiency of the qPCR assay ranged from 98% to 109%. The threshold cycle (Ct) values and melting temperatures of selected qPCR reactions are listed in Table 8. The standard series displayed a gradient of increasing Ct values from template concentration 1 to 1×10⁻⁴ ng/μl, while there was no detection for 1×10⁻⁶ or 1×10⁻⁷ ng/μl. Among the three replicates of 1×10⁻⁵ ng/μl, one showed detection while the other two did not. Since the theoretical detection limit was 0.8×10⁻⁵ ng/μl 3C genomic DNA in the template (equivalent to one copy of target gene per reaction), it is reasonable that there was no consistent detection when the template concentration of 3C genomic DNA was 1×10⁻⁵ ng/μl or less. Since the error of pipetted amount of target DNA is very high when the target concentration is near one copy per reaction, any detection event by Ct greater than 35.1 (the Ct of that standard rep at 1×10⁻⁵ ng/μl) should be considered only qualitative instead of quantitative.

To test the specificity of this assay within the Cryptococcus flavescens species, qPCR was also run on the 1 ng/μl genomic DNA templates of six other C. flavescens strains than 3C (Table 8). The sequences of strains Y-7372, YB-328 and YB-744 differ from 3C by two base pairs at each primer binding site (FIG. 18). These three strains showed much higher Ct values than 3C genomic DNA at the same template concentration. The Ct difference of around 15 (equal to approximately 3×10⁴-fold difference in starting quantity) between 3C and the aforementioned three strains can well differentiate 3C from them. In contrast, strains Y-7373, YB-601 and YB-602 showed 100% sequence identity to 3C across the whole amplicon (FIG. 18). These three strains could not be differentiated from 3C by the assay primer pair h31.2 (Table 8) or any of the other 13 tested primer pairs since they showed comparable Ct values to 3C for all 14 primer pairs. Therefore, what the assay quantifies is the population of 3C-like C. flavescens, including 3C as well as Y-7373, YB-601 and YB-602, instead of 3C population only.

TABLE 8
Sensitivity and specificity of the qPCR assay targeting the putative hsp70 gene

| | | Concentration | | Melting |
|---|---|---|---|---|
| Template | Strain name or | (ng/μl in | | temperature |
| Nature | other identifier | template) | Threshold Cycle^{a} | (° C.)^{b} |
| Genomic DNA, | 3C | 1 | 19.4 | 87.3 |
| mean of 3 | | 10⁻¹ | 22.5 | 87.0 |
| replicates of | | | | |
| standard series | | 10⁻² | 26.2 | 87.0 |
| | | 10⁻³ | 29.7 | 87.3 |
| | | 10⁻⁴ | 33.0 | 87.3 |
| | | 10⁻⁵ | 35.1 (1 rep) or n.d. | 87.0 (1 rep) or |
| | | | (2 reps) | n.a. (2 reps) |
| | | 10⁻⁶ | n.d. | n.a. |
| | | 10⁻⁷ | n.d. | n.a. |
| Genomic DNA | Y-7372 | 1 | 34.0 | 87.0 |
| | Y-7373 | 1 | 17.3 | 87.0 |
| | YB-328 | 1 | 34.8 | 87.0 |
| | YB-601 | 1 | 19.0 | 87.5 |
| | YB-602 | 1 | 18.7 | 87.5 |
| | YB-744 | 1 | 35.0 | 87.0 |
| DNA extract of | Inoculated with 3C | — | 26.5 | 87.5 |
| wheat head from | Not inoculated with | — | n.d. | n.a. |
| field^{c} | 3C | | | |

^{a}Threshold Cycle was not detected (n.d.) by the software when fluorescence signal was below the threshold at the end of 40-cycle amplification.
^{b}Melting temperature was not applicable (n.a.) for the samples for which the detection software reported no peak of 87.0 or 87.5° C. (the only reported peak for standard series 1 through 10⁻⁴ ng/μl in template).
^{c}The templates of the field samples are the 1:20 diluted DNA extracts from a 3C-inoculated wheat head 44 days post inoculation and from any non-inoculated head at a distance of 25.8 m to the nearest inoculated area on the day of inoculation.

Dynamics of 3C-Like C. flavescens Population in Areas Inoculated with 3C.

The population density of 3C-like C. flavescens on wheat heads, represented by the target gene copy number per gram wheat head tissue, fluctuated over time in the areas inoculated with 3C (FIG. 15). The samples at one day post inoculation (DPI) showed significantly lower population than those at and after 10 DPI. This indicates that 3C cells multiply on wheat heads between 1 and 10 DPI, leading to the qPCR detection of population growth. There were trends of population changes from 0 to 1 DPI and from 10 to 44 DPI, but they were not statistically significant based on an α=0.05. \

3C-like C. flavescens population on the majority of the samples was between 10^{5.5} and 10^{6.0} copies of target gene per gram wheat head tissue immediately post inoculation (0 DPI) and was between 10^{4.5} and 10^{5.5} on 1 DPI. Subsequently, population increased to between 10^{6.0} and 10^{6.5} by the end of the season. Given that the inoculation rate was 10^{6.3} to 10^{6.8} 3C cells per square centimeter and that the horizontal section of a wheat head was around one square centimeter, qPCR-detected population was maintained at a level that was slightly lower than, but still comparable to, the inoculation rate throughout the growing season.

Dispersal of 3C to the Non-Inoculated Area of Field.

There was a trend that 3C-like population decreased as the distance from inoculated area increased, displayed by the percentages of samples above a threshold (3.3×10⁴ gene copy per gram wheat head) around the minimal population density necessary to show biocontrol efficacy (FIG. 7A). This trend is also demonstrated when the samples of different population levels are shown as fractions of total (FIG. 7B). Furthermore, the trend that populations in non-inoculated area increased over time from 1 to 44 DPI was observed (FIGS. 7A and 7B). This indicates that 3C was dispersed stochastically from the inoculated area to non-inoculated area over time. By the last sampling in the growing season (44 DPI), the majority of samples from non-inoculated area harbored 3.3×10³ to 3.3×10⁵ target gene copies per gram wheat head of the hsp70 marker.

There were a few samples (i.e. 5 of 36) positive for 3C-like C. flavescens in the non-inoculated area at 0 DPI. However the level detected was 3.3×10² to 3.3×10³ target gene copies of the target per gram wheat head, an amount around the detection limit of the assay. And, none of the non-inoculated samples at 1 DPI (i.e. 0 of 35) scored positive for 3C-like populations. These data indicate that colonization of wheat heads by native C. flavescens was generally infrequent and, when it did occur, never reached 10⁴ gene copies per wheat head. Thus, the majority of qPCR-detected populations in this experiment were interpreted as arising from 3C inoculation, and this is why the aforementioned population dynamics over distances and time reflect the dispersal pattern of 3C.

The Effect of Sample Collection Activity on 3C Dispersal.

To test whether sample collectors walking throughout the field contributed to the apparent dispersal of 3C, additional wheat head samples were collected from borders of wheat-grown area in Field One on 10 DPI. Since sample collectors walked along the fallow walkways in between the wheat-grown area, it was possible that they brought 3C from inoculated area to non-inoculated area. It was hypothesized that if sample collection activity did contribute to 3C dispersal, the wheat heads at the borders of wheat-grown area neighboring the walkways should show higher 3C population density than those deeper inside of wheat-grown area do. The samples relevant to this hypothesis were collected from the eastern side of the field (see FIG. 5 for details on sampling locations) since the sample collectors were required to exit the field from that side after they collected samples from inoculated area. There was no significant difference (n>7, P>0.78) between the population densities on the wheat heads from the borders and inside of wheat-grown area, indicating that the sample collection activity did not significantly contribute to the dispersal of 3C in this experiment.

The Effect of Prevailing Wind Direction on 3C Dispersal.

To test whether the direction of prevailing wind (from west to east) affected the apparent patterns of 3C dispersal, the wheat head samples from the western and eastern sides of Field One were compared. The samples from different location groups (see FIG. 5 for details on location groups) and/or at different time points were compared separately. It was hypothesized that the samples from the eastern side of the field should have shown higher 3C population density than those from the western side did if the prevailing wind did play an important role in 3C dispersal. The differential patterns of 3C population density were not consistent across different location groups and sampling time points, and there was no obvious trend towards either side of the field. This indicates that the direction of prevailing wind did not have a significant effect on the patterns of 3C dispersal in this experiment. This is consistent with the fact that 3C is a yeast without known production of wind-dispersed spores, therefore may not be easily dispersed by wind alone. Though 3C may be better dispersed by wind-blown rain, rainfall events may not correlate with prevailing wind direction.

Overwintering Persistence of 3C-Like C. flavescens.

To test whether 3C-like C. flavescens can persist in the field over the winter, the three inoculated areas of Field One were sampled in the winter to early spring of 2012. The data showed that population could still be detected in inoculated area by 6 to 9 months postharvest (FIG. 16). Since there was no significant difference among the same type of samples in different inoculated areas, the data points from the three inoculated areas were combined for the comparison between sample types. The non-rotting wheat stalk samples showed significantly higher levels of population (mostly 10⁴ to 10⁵) per gram weight than the composite samples of soil, living weeds and rotting plant materials (mostly 10^{2.5} to 10^{3.5}) did (FIG. 16). Population on overwintering residues was lower than that on wheat heads by the end of growing season, which was mostly above 10⁶ target gene copies per gram sample at 44 DPI.

Population Levels of 3C-Like C. flavescens Around Harvest and During Storage.

Additional wheat head and grain samples were collected from another field (Field Two) at different time points around harvest. Population levels on grains were repetitively measured by qPCR after periods of storage. The inoculated samples showed generally higher population than the non-inoculated samples for both sample types at different time points (FIG. 17). Most of the samples from inoculated plots, including the heads and the threshed grains, showed detection of higher than 10^{3.5} and up to 10^{6.7} target gene copies per gram. There was a trend that population on wheat heads increased from 11 days pre-harvest to 1 day pre-harvest in both inoculated and non-inoculated plots. For the samples collected from non-inoculated plots, population was significantly lower on the threshed grains of 14 days postharvest (mostly around 10^{3.5} target gene copies per gram) than on the heads of one day pre-harvest (mostly between 10^{4.0} and 10^{4.5} target gene copies per gram) (P=0.037 for Tukey's test). The inoculated samples showed a trend of such difference but it wasn't significant (P=0.22 for Tukey's test). There was a trend of progressive population decrease on threshed grains from both inoculated and non-inoculated plots during storage from 14 to 64 and to 155 days postharvest (FIG. 17).

Discussion

This example describes the development of a qPCR assay to monitor the dispersal and persistence of 3C-like Crypotococcus flavescens on field-grown wheat and, more broadly, in the environment. This assay enabled the detection with clear differential between the inoculated and non-inoculated wheat head samples (Table 8, last two rows). And it can be reasonably applied to multiple sample types, since it gave meaningful differential results within each of the sample types tested in this study. The sensitivity of the present assay was comparable to other related qPCR assays. Previous studies using qPCR to detect fungal pathogens on wheat showed minimum detections at the magnitude of 10¹ gene copies per reaction and 10⁴ gene copies or 1 to 10³ ng genomic DNA per gram wheat tissue, which were comparable to those in the present assay (12.5 gene copies per reaction and 10³ to 10⁴ gene copies or 0.01 to 0.1 ng genomic DNA per gram wheat tissue) (Table 8, FIG. 7B, and FIG. 16). The present qPCR assay may not be as sensitive as culture-based quantification of 3C, which could detect 10 to 100 cells per gram wheat tissue. However, this qPCR assay is still superior to culture-based quantification in that the former has inter-strain specificity while the latter cannot differentiate among subspecies of C. flavescens based on morphology. Also the qPCR assay enables long-term storage of samples prior to quantification without underestimating the populations due to die-off of cells during storage.

The qPCR assay showed intra-species specificity to four 3C-like C. flavescens strains (3C, Y-7373, YB-601 and YB-602) over the other three strains (Table 8). No further discrimination by Threshold Cycle (Ct) value nor melting temperature can be achieved among 3C-like strains, which is due to the high sequence similarity among them. Sequence difference between 3C and the three 3C-like strains was actually 0 to 1 bp across a total of more than 5500 bp of DNA (including ˜2000 bp putatively non-coding sequences) across 9 conserved regions, including all the 7 regions used for primer design in this study. Since mismatch by one base pair at primer binding site only gave rise to a Ct difference of ˜1.5 for one of the tested primers, it is almost impossible to differentiate among 3C-like C. flavescens through amplification from those conserved regions. It may be possible to design primers targeting more variable regions to achieve “3C-only” specificity. However, such a strategy may sacrifice sensitivity because the higher natural mutation rates in variable regions may result in loss of ability to detect 3C progenies that have mutations in primer binding sites. Furthermore, the possibility cannot be excluded that some 3C-like strains may have originated from the same population as 3C in the near past and were named as different strains simply due to separate isolation events. The biocontrol efficacy against Fusarium head blight did not differ significantly among 3C and other 3C-like strains. Therefore, further refinement of the assay to gain additional specificity to the isolate 3C will not provide much practical value.

Previous studies demonstrate that applying 3C inoculum of approximately 3×10⁸ CFU/ml at the rate of about 10⁶ CFU per square centimeter resulted in 10¹ to 10² CFU/g glume tissue at 16 h post inoculation and 10⁵ to 10⁶ CFU at 256 to 280 h post inoculation. In those studies, strain CFU of strain 3C decreased by a magnitude of 10⁴ to 10⁵ compared to the application rate in the 16 h post inoculation. In contrast, there was only an approximately 10-fold decrease in 3C DNA population during the one day following inoculation in the present study (FIG. 15). This dramatic difference is due to that most of the 3C DNA detected on 1 DPI in the present study belonged to non-viable cells or was already released from lysed cells. However, 3C CFU level increased to the similar level to the application rate 10 days later in those previous studies and was also the case in the present study. This indicates that most of the 3C DNA detected on 10 DPI in the present study belonged to viable cells.

Previously developed qPCR assays of other wheat-associated eukaryotes showed inter-species specificities. The present assay showed a certain level of inter-strain specificity within the species of Cryptococcus flavescens. Specifically, assays of six other isolates of C. flavescens indicated that the species encompassed two genotypic groups based on the amplification efficiency of standard amounts of DNA template (Table 8). Even though all 3C-like isolates showed similar amplification efficiency, detection of native populations of 3C's close relatives was minimal in this study. This is because no detection events were noted across nearly all the non-inoculated samples on 0 and 1 DPI while the inoculated samples all showed clear detection (FIG. 7). If there had been a significant interference, those non-inoculated samples would have shown more frequent and higher levels of detection of amplification. Second, the level of detection generally decreased as the distances from inoculated plots increase (FIG. 7). If there had been a significant interference, the levels of detection would have been generally the same or more random across the field. These data indicate that native populations of 3C-like C. flavescens are relatively rare on wheat heads between flowering and harvest.

In some studies on the population dynamics of plant-associated bacteria using culture-based methods, decreased bacterial population on flowers or leaves of host plants later in the experimental time courses were observed. In a study using qPCR for population quantification, the total population of three Fusarium species died off significantly on spring wheat stubble residue in six months after the harvest in summer. In the previous studies on 3C colonization of wheat using CFU-based quantification, slight die-off of 3C population was observed between 184 and 256 to 280 h after wheat flowering and inoculation in the 2009 run but not in the 2010 run. The present study showed trends of population decrease on wheat heads from 0 to 1 DPI (FIG. 15) and on threshed grains during storage (FIG. 16). The die-off of 3C-like C. flavescens (the majority of which we believe was 3C) in the present study is consistent with the observations in previous studies. This indicates that 3C will not pose a threat to ecological balance or food safety through prolonged multiplication.

The dispersal pattern of 3C across the wheat field in this study resembles those reported previously for several fungal pathogens on wheat. In such studies, at each given time point, the microorganism population decreased steeply from artificially inoculated area (source) to non-inoculated area (sink) near the source, while decreased gently over farther distances across the sink, especially at early time points. In the present study, there were obvious increases in 3C population at the same distances in the sink over time (FIG. 7), which is more similar to the dispersal pattern of a wheat pathogen on pure stand of susceptible cultivar than to on a mixture of susceptible and resistant cultivars. This indicates that wheat is a compatible host to 3C, which is consistent with the fact that 3C is able to colonize wheat heads effectively. And the present assay is robust enough to successfully demonstrate this pattern.

In previous studies, qPCR assays for quantification of wheat pathogens have been used to evaluate disease severity and to monitor and predict disease development. The present qPCR assay can be used to determine environmental fate of 3C-like C. flavescens as biopesticides. Specifically, dispersal and persistence of 3C-like C. flavescens can be investigated throughout the wheat production system at various geographic locations over multiple growing seasons. It can also be used to study the effects of common field abiotic and biotic factors of concern on 3C population dynamics in order to optimize the formulation of 3C-based biopesticides. Further, it can be used to investigate the association between 3C population dynamics and biocontrol efficacy of 3C against Fusarium head blight under various conditions.

### Example 6

### Non-Limiting Examples of Applications

One or more strains of Cryptococcus flavescens, one or more microbial antagonists, a composition, or groups of primer pairs described herein may be provided in the form of a kit. In some embodiments, the kit provides one or more strains of C. flavescens, microbial antagonists, or compositions for application to the seed head of a plant, most preferably a cereal plant.

In other embodiments, the kit provides a group of primer pairs designed specifically to amplify β-tubulin, chitin synthase 1, EF1, and/or hsp70. The kits may also provide one or more containers filled with one or more necessary PCR reagents, including but not limited to dNTPs, reaction buffer, Taq polymerase, and RNAse-free water. Optionally associated with such container(s) is a notice in the form prescribed by a governmental agency regulating the manufacture, use or sale of the group of primer pairs and associated reagents, which notice reflects approval by the agency of manufacture, use or sale for research use.

The kits may include appropriate instructions for preparing supplied C. flavescens strains, microbial antagonists, and compositions for application. Instructions may further describe appropriate application techniques and concentrations of prepared C. flavescens strains, microbial antagonists, and compositions. When group of primer pairs are included in the kit, the kit may include appropriate instructions for preparing, executing, and analyzing qPCR to determine the genotype of a C. flavescens strain using the primer pairs included in the kit. The instructions may be in any suitable format, including, but not limited to, printed matter, videotape, computer readable disk, or optical disc.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiment disclosed herein contemplated for carrying out this invention, but that the invention will include all embodiments falling within the scope of the claims.

It is intended that the specification and examples be considered as exemplary only, with a true scope and spirit of the invention being indicated by the following claims.

What is claimed is:

1. A method for suppressing Fusarium head blight in a cereal plant comprising:
a) identifying a genotype for a Cryptococcus flavescens strain by qPCR and/or by sequence identity; and
b) applying to a seed head of said plant an effective amount of one or more microbial antagonists, wherein the one or more microbial antagonist is a Genotype A Cryptococcus flavescens strain or Genotype B Cryptococcus flavescens strain, as identified in step a), wherein the one or more microbial antagonists is not Cryptococcus flavescens 3C, which has been deposited under NRRL accession no. Y-50378, or Cryptococcus flavescens 4C, which has been deposited under NRRL accession no. Y-50379.

2. The method of claim 1, wherein the genotype for a Cryptococcus flavescens strain is determined by quantitative PCR (qPCR), comprising the steps:
a) performing qPCR on extracted genomic DNA utilizing one or more primer pairs sharing at least 90% sequence identity with the primer pairs of Table 1 (SEQ ID NOs: 45-72), wherein sequence identity is determined for each individual forward or reverse primer; and
b) identifying a Cryptococcus flavescens strain as Genotype A or Genotype B, wherein:
(1) the Cryptococcus flavescens strain is identified as Genotype A when qPCR results in: a threshold cycle value of 17-21 when using the btub.1, btub.2, or EF1.2 primer sets; a threshold cycle value of 18-20 when using the h22.1, h30.2, or h31.1 primer sets; a threshold cycle value of 17-20 when using the h31.2 primer set; or a threshold cycle value of 12-15 when using the I2.1 primer set; and
(2) the Cryptococcus flavescens strain is identified as Genotype B when qPCR results in a threshold cycle value of 31-33 when using the btub.1 primer set; a threshold cycle value of 32-35 when using the btub.2 primer set; a threshold cycle value of 24-26 when using the EF1.2 primer set; a threshold cycle value of 22-24 when using the h22.1 or h31.1 primer sets; a threshold cycle value of 22-23 when using the h30.2 primer set; a threshold cycle value of 34-35 when using the h31.2 primer set; or a threshold cycle value of 15-18 when using the I2.1 primer set.

3. The method of claim 2, wherein the extracted genomic DNA is collected from one or more cereal grass plants prior to application of one or more microbial antagonists to the one or more cereal grass plants.

4. The method of claim 2, wherein the extracted genomic DNA is collected from one or more cereal grass plants following application of one or more microbial antagonists to the one or more cereal grass plants.

5. The method of claim 2, wherein the extracted genomic DNA is collected from an in vitro Cryptococcus flavescens culture.

6. The method of claim 1, wherein the genotype for a Cryptococcus flavescens strain is determined by sequence identity, comprising the steps:
a) sequencing extracted genomic DNA of a target Cryptococcus flavescens;
b) comparing the sequence determined in step a) to known homologous sequences of other Cryptococcus flavescens strains; and
c) identifying a Cryptococcus flavescens strain as Genotype A or Genotype B, wherein:
(1) the Cryptococcus flavescens strain is identified as Genotype A when the sequence of the extracted genomic DNA of the target Cryptococcus flavescens shares: 100% sequence identity at the β-tubulin gene to SEQ ID NO:1; between 95% and 100% sequence identity at the chitin synthase 1 gene to SEQ ID NOs: 2, 3, 4, 5, or 6; between 99% and 100% sequence identity at the EF1 gene to SEQ ID NOs: 7, 8, 9, 10, or 11; between 99% and 100% sequence identity at the hsp70 gene to SEQ ID NOs: 12, 13, 14, 15, or 16; or between 99% and 100% sequence identity at the cs22 target locus to SEQ ID. NOs: 85, 86, 87, 88, or 89; and
(2) the Cryptococcus flavescens strain is identified as Genotype B when the sequence of the extracted genomic DNA of the target Cryptococcus flavescens shares: between 99% and 100% sequence identity at the β-tubulin gene to SEQ ID NOs: 17, 18, 19, 20, 21, 22 or 23; between 97% and 100% sequence identity at the chitin synthase 1 gene to SEQ ID NOs: 24, 25, 26, 27, 28, 29 or 30; between 99% and 100% sequence identity at the EF1 gene to SEQ ID NOs: 31, 32, 33, 34, 35, 36, or 37; between 99% and 100% sequence identity at the hsp70 gene to SEQ ID NOs: 38, 39, 40, 41, 42, 43 or 44; or 100% sequence identity at the cs22 target locus to SEQ ID NO: 90.

7. The method of claim 6, wherein the extracted genomic DNA is collected from one or more cereal grass plants prior to application of one or more microbial antagonists to the one or more cereal grass plants.

8. The method of claim 6, wherein the extracted genomic DNA is collected from one or more cereal grass plants following application of one or more microbial antagonists to the one or more cereal grass plants.

9. The method of claim 6, wherein the extracted genomic DNA is collected from an in vitro Cryptococcus flavescens culture.

10. The method of claim 1, wherein at least one of the one or more microbial antagonists is a Genotype A Cryptococcus flavescens strain.

11. The method of claim 1, wherein at least one of the one or more microbial antagonists is a Genotype B Cryptococcus flavescens strain.

12. The method of claim 1, wherein at least one of the one or more microbial antagonists is selected from the group of C. flavescens strains consisting of: Y-7373; YB-601; YB-602; Y-7377; Y-7372; Y-7375; Y-7374; Y-7376; YB-328; Y-7379; and YB-744.

13. The method of claim 1, wherein at least one of the one or more microbial antagonists is tolerant to prothioconazole.

14. The method of claim 1, wherein in addition to the one or more microbial antagonists being applied to a seed head of a cereal plant, Cryptococcus flavescens 3C, which has been deposited under NRRL accession no. Y-50378, and Cryptococcus flavescens 4C, which has been deposited under NRRL accession no. Y-50379, is applied to the cereal plant.

15. The method of claim 1, wherein the one or more microbial antagonists are applied to the seed head prior to a hard dough stage of development.

16. The method of claim 1, wherein the one or more microbial antagonists are applied to the seed head during flowering.

17. The method of claim 1, wherein the one or more microbial antagonists are applied to the seed head prior to flowering.

18. The method of claim 1, wherein the cereal plant is selected from the group consisting of: wheat; and barley.

19. The method of claim 1, wherein the effective amount of at least one microbial antagonist is an amount sufficient to reduce the level of Fusarium head blight relative to that in a corresponding untreated control.

20. The method of claim 1, wherein application of an effective amount of one or more microbial antagonists to the seed head of a cereal plant comprises spraying the one or more microbial antagonists onto the cereal plant.

21. The method of claim 20, wherein the method of spraying is selected from the group consisting of: spraying through a sprinkler irrigation system; aerial spray application; ground-based spray application.

22. The method of claim 1, wherein the effective amount of the one or more microbial antagonists is between about 10⁴-10⁹ CFU/ml applied at a rate of about 10⁵-10⁶ CFU/cm².

23. The method of claim 22, wherein the effective amount of the one or more microbial antagonists is about 1.5×10⁹ CFU/ml applied at a rate of about 2×10⁶ to 6×10⁶ CFU/cm².

24. The method of claim 22, wherein the effective amount of the one or more microbial antagonists is about 2.3×10⁸ CFU/ml applied at a rate of about 2×10⁵ CFU/cm².

25. The method of claim 22, wherein the effective amount of the one or more microbial antagonists is about 3×10⁸ CFU/ml at a rate of about 10⁶ CFU/cm².

26. The method of claim 1, wherein application of an effective amount of the one or more microbial antagonists to the seed head of a cereal plant occurs at temperatures between about 5 to 35° C.

27. The method of claim 1, wherein application of an effective amount of the one or more microbial antagonists to the seed head of a cereal plant occurs at temperatures between about 15 to 30° C.

28. The method of claim 1, wherein the one or more microbial antagonists are substantially biologically pure.

29. The method of claim 1, wherein two or more microbial antagonists are applied to the seed head of the cereal plant.

30. The method of claim 29, wherein the two or more microbial antagonists are applied simultaneously.

31. The method of claim 29, wherein the two or more microbial antagonists are applied separately.

32. The method of claim 29, wherein at least two microbial antagonists are Genotype A Cryptococcus flavescens strains.

33. The method of claim 29, wherein at least two microbial antagonists are Genotype B Cryptococcus flavescens strains.

34. The method of claim 29, wherein a first microbial antagonist is a Genotype A Cryptococcus flavescens strain and a second microbial antagonist is a Genotype B Cryptococcus flavescens strain.

35. The method of claim 29, wherein at least one microbial antagonist is selected from a first group consisting of: Genotype A Cryptococcus flavescens strains; and Genotype B Cryptococcus flavescens strains, and at least one microbial antagonist is selected from a second group consisting of: Cryptococcus flavescens 3C, which has been deposited under NRRL accession no. Y-50378; and Cryptococcus flavescens 4C, which has been deposited under NRRL accession no. Y-50379.

36. The method of claim 1, further comprising applying one or more fungicides to the cereal plant.

37. The method of claim 36, wherein the fungicide is prothioconazole.

38. The method of claim 36, wherein the one or more fungicides are applied to the cereal plant at a time selected from the group consisting of: prior to application of the one or more microbial antagonists; simultaneously with the application of the one or more microbial antagonists; and subsequent to the application of the one or more microbial antagonists.

39. A kit comprising one or more microbial antagonists of claim 1.

40. A kit comprising one or more primer pairs sharing at least 90% sequence identity with the primer pairs of Table 1 (SEQ ID NOs: 45-72), wherein sequence identity is determined for each individual forward or reverse primer.

41. A method for identifying the genotype of a Cryptococcus flavescens strain comprising:
a) performing quantitative PCR (qPCR) on extracted genomic DNA utilizing one or more primer pairs sharing at least 90% sequence identity with the primer pairs of Table 1 (SEQ ID NOs: 45-72), wherein sequence identity is determined for each individual forward or reverse primer; and
b) identifying a Cryptococcus flavescens strain as Genotype A or Genotype B, wherein:
(1) the Cryptococcus flavescens strain is identified as Genotype A when qPCR results in: a threshold cycle value of 17-21 when using the btub.1, btub.2, or EF1.2 primer sets; a threshold cycle value of 18-20 when using the h22.1, h30.2, or h31.1 primer sets; a threshold cycle value of 17-20 when using the h31.2 primer set; or a threshold cycle value of 12-15 when using the I2.1 primer set; and
(2) the Cryptococcus flavescens strain is identified as Genotype B when qPCR results in a threshold cycle value of 31-33 when using the btub.1 primer set; a threshold cycle value of 32-35 when using the btub.2 primer set; a threshold cycle value of 24-26 when using the EF1.2 primer set; a threshold cycle value of 22-24 when using the h22.1 or h31.1 primer sets; a threshold cycle value of 22-23 when using the h30.2 primer set; a threshold cycle value of 34-35 when using the h31.2 primer set; or a threshold cycle value of 15-18 when using the I2.1 primer set.

42. The method of claim 41, wherein the extracted genomic DNA is collected from one or more cereal grass plants prior to application of one or more microbial antagonists to the one or more cereal grass plants.

43. The method of claim 41, wherein the extracted genomic DNA is collected from one or more cereal grass plants following application of one or more microbial antagonists to the one or more cereal grass plants.

44. The method of claim 41, wherein the extracted genomic DNA is collected from an in vitro Cryptococcus flavescens culture.

45. A method for identifying the genotype of a Cryptococcus flavescens strain comprising:
a) sequencing extracted genomic DNA of a target Cryptococcus flavescens;
b) comparing the sequence determined in step a) to known homologous sequences of other Cryptococcus flavescens strains; and
c) identifying a Cryptococcus flavescens strain as Genotype A or Genotype B, wherein:
(1) the Cryptococcus flavescens strain is identified as Genotype A when the sequence of the extracted genomic DNA of the target Cryptococcus flavescens shares: 100% sequence identity at the β-tubulin gene to SEQ ID NO:1; between 95% and 100% sequence identity at the chitin synthase 1 gene to SEQ ID NOs: 2, 3, 4, 5, or 6; between 99% and 100% sequence identity at the EF1 gene to SEQ ID NOs: 7, 8, 9, 10, or 11; between 99% and 100% sequence identity at the hsp70 gene to SEQ ID NOs: 12, 13, 14, 15, or 16; or between 99% and 100% sequence identity at the cs22 target locus to SEQ ID. NOs: 85, 86, 87, 88, or 89; and
(2) the Cryptococcus flavescens strain is identified as Genotype B when the sequence of the extracted genomic DNA of the target Cryptococcus flavescens shares: between 99% and 100% sequence identity at the β-tubulin gene to SEQ ID NOs: 17, 18, 19, 20, 21, 22 or 23; between 97% and 100% sequence identity at the chitin synthase 1 gene to SEQ ID NOs: 24, 25, 26, 27, 28, 29 or 30; between 99% and 100% sequence identity at the EF1 gene to SEQ ID NOs: 31, 32, 33, 34, 35, 36, or 37; between 99% and 100% sequence identity at the hsp70 gene to SEQ ID NOs: 38, 39, 40, 41, 42, 43 or 44; or 100% sequence identity at the cs22 target locus to SEQ ID NO: 90.

46. The method of claim 45, wherein the extracted genomic DNA is collected from one or more cereal grass plants prior to application of one or more microbial antagonists to the one or more cereal grass plants.

47. The method of claim 45, wherein the extracted genomic DNA is collected from one or more cereal grass plants following application of one or more microbial antagonists to the one or more cereal grass plants.

48. The method of claim 45, wherein the extracted genomic DNA is collected from an in vitro Cryptococcus flavescens culture.


SEQUENCE LISTING (100 sequences)

SEQ ID NO: 1 - DNA, 534 bp, Cryptococcus flavescens
ggaacgatggactcgatccggggtgggccgctcggctcgctcttccgacctgacaacttgtgagcttgggtccagatagcgtccagagtgtaccaggggctgacggtagcgtgtttggacagagcggagcgggaaacaactgggcgaagggtcactacaccgagggtgcgtgcgcggttccatcataccccacctctgtctgcttacagctcgcccactgaccccaggtgccgagctggtcgactcggtcctcgacgtcgtccgacgagaggcggagaacacggactgcctccagggtttccagatcacccactcgctcggtggaggaaccggagccggtatgggaacattgttgatctccaagatcagggaggagtacccggaccgaatgatgtgcaccttctcggtcgtcccctcgcccaaggtgcgtccaaggctgccgtggtgttgtacgatctaacccgcaggtgtccgacaccgttgtcgagccgtacaacgcgacgctctcggtccaccagctcgttgagaactctg

SEQ ID NO: 2 - DNA, 332 bp, Cryptococcus flavescens
agggagaagatcacgttgtgctggattgcgtgtctgatgggtgcggtggtgggctttgcgacgatgggactgcagagggcattgtgtcctgagggttcgacgagtatattccggcgactgggggatgatcagagtgagtcgaggttgtggctgtcgttcgattgtgggcggtgggcggagggcggaggcatgggtgaagatagcggaaagcagcagagttgacatgcttggactagcggaggtggacatggcgattagcacctggaaagtccgctgtcggaggacgatcttgagcgcgcagcggaagaggcagatgagcgcgcgaagcctga

SEQ ID NO: 3 - DNA, 332 bp, Cryptococcus flavescens
agggagaagatcacgttgtgctggattgcgtgtctgatgggtgcggtggtgggctttgcgacgatgggactgcagagggcattgtgtcctgagggttcgacgagtatattccggcgactgggggatgatcagagtgagtcgaggttgtggctgtcgttcgattgtgggcggtgggcggagggcggaggcatgggtgaagatagcggaaagcagcagagttgacatgcatggactagcggaggtggacatggcgattagcacctggaaagtccgctgtcggaggacgatcttgagcgcgcagcggaagaggcagatgagcgcgcgaagcctga

SEQ ID NO: 4 - DNA, 332 bp, Cryptococcus flavescens; identical to SEQ ID NO: 2

SEQ ID NO: 5 - DNA, 332 bp, Cryptococcus flavescens; identical to SEQ ID NO: 2

SEQ ID NO: 6 - DNA, 334 bp, Cryptococcus flavescens
agggagaagatcacgttgtgctggattgcgtgtctgatgggtgcggtggtgggcttcgcgacgatgggactgcagagggcgctgtgtccggagggctcgacgagcatcttccggcggttaggggatgatcagagtgagtcgaggttgtggctgtcgttcgattgtgggcggtgggcggagggcggaggcatgggtgaagatagcggaaagcagcagagttgacatgcttggactagcggaggtggacatggcggttaccacctggaaagtccgctgtcggagaggacgatcttgagcgcgcagcggaagaggcagatgagcgcgcgaagcctga

SEQ ID NO: 7 - DNA, 597 bp, Cryptococcus flavescens
taagaccctccttgacgccatcgacgccatcgacccccctcagcgacccaccgacaagccgctccgacttcccctccaggatgtctacaagatcggtggtatcggcacagtgcccgtcggccgagtcgagaccggtgtgatcaagggagggtgagtgatagttttttgccacccgccacccgccaaccgtcacccgtcacctgcgtgactcccttgacccttcgtgtacgtccttgcagacgctgatatcagaatggttgttaccttcgccccgaccaacgtcaccaccgaggtcaagtccgtcgagatgcaccacgagcaggttagtcccgccttccgccacattttgccactcaccccttccgccccttcgctttctttccttcgctgacatccagatccccgagggtctcccgggagacaacgtcggcttcaacgtcaagaacgtctccatcaaggacatccgacgaggaaacgtctgctccgactcgaagaacgaccccgctaaggaggccgcttccttcaacgcccaggtcattgtcctcaaccacccgggacagatcggtgccggctacaccccggtccttgactgccaca

SEQ ID NO: 8 - DNA, 597 bp, Cryptococcus flavescens; identical to SEQ ID NO: 7

SEQ ID NO: 9 - DNA, 597 bp, Cryptococcus flavescens
taagaccctccttgacgccatcgacgccatcgacccccctcagcgacccaccgacaagccgctccgacttcccctccaggatgtctacaagatcggtggtatcggcacagtgcccgtcggccgagtcgagaccggtgtgatcaagggagggtgagtgatagttttttgccacccgccacccgccaaccgtcacccgtcacctgcgtgactcccttgacccttcgtgtacgtccttgcagacgctgatatcagaatggttgttaccttcgccccgaccaacgtcaccaccgaggtcaagtccgtcgagatgcaccacgagcaggttagtcccgccttccgccacattttgccactcaccccttctgccccttcgctttctttccttcgctgacatccagatccccgagggtctcccgggagacaacgtcggcttcaacgtcaagaacgtctccatcaaggacatccgacgaggaaacgtctgctccgactcgaagaacgaccccgctaaggaggccgcttccttcaacgcccaggtcattgtcctcaaccacccgggacagatcggtgccggctacaccccggtccttgactgccaca

SEQ ID NO: 10 - DNA, 597 bp, Cryptococcus flavescens; identical to SEQ ID NO: 9

SEQ ID NO: 11 - DNA, 597 bp, Cryptococcus flavescens
taagaccctccttgacgccatcgacgccatcgacccccctcagcgacccaccgacaagccgctccgacttcccctccaggatgtctacaagatcggtggtatcggcacagtgcccgtcggccgagtcgagaccggtgtgatcaagggagggtgagtgatagttttttgccacccgccacccgccaagcgtcacccgtcacctgcgtgactcccttgacccttcgtgtacgtccttgcagacgctgatatcagaatggttgttaccttcgccccgaccaacgtcaccaccgaggtcaagtccgtcgagatgcaccacgagcaggttagtcccgccttccgccacattttgccactcaccccttccgccccttcgctttctttccttcgctgacatccagatccccgagggtctcccgggagacaacgtcggcttcaacgtcaagaacgtctccatcaaggacatccgacgaggaaacgtctgctccgactcgaagaacgaccccgctaaggaggccgcttccttcaacgcccaggtcattgtcctcaaccacccgggacagatcggtgccggctacaccccggtccttgactgccaca

SEQ ID NO: 12 - DNA, 698 bp, Cryptococcus flavescens
tacccgatctcttcgatcatccgtcgctcgcccaagcgcccttcgagctgccaacgctgctgtacgtctcgtttgtctcgctcgatcgccccaactcatggcgctaacattttgcagcgatgcacccccctaatcagcaagcgattcaacagtggcaaggtctctggacccgtcatcgggtacgcctgccccccgtctgctatacaagattcgacgagctcacagtcagtattgacttgggaacgactaactcctgtgtctcgtgagctatttctttgcggctgctgtcttgcggtcgcttaacatcagcatcttcgagaacggcggtgccaaggtccttgagaacgcggagggtgcccgaaccaccccatcggttgttgcctttaccaagggtgcgcatcgcgatttacgaagtggcaacacgctgacgtacagatggcgagcgattagtcggtcaaccggctcgacgacaggccgttgtcaacggtgaacacaccatctttgcctccaagcggtaagccggcaaatccgcctaagatgagccgcagaactgatcccctagactgatcggacgaaagttcaccgacgccgaggtccagaaggacatcaagaacgtcccgtacaagattgtcgcccactctaacggtgacgcttgggtcgaggctcgaggacagaagtactcgccatcgcaaattggt

SEQ ID NO: 13 - DNA, 698 bp, Cryptococcus flavescens; identical to SEQ ID NO: 12

SEQ ID NO: 14 - DNA, 698 bp, Cryptococcus flavescens; identical to SEQ ID NO: 12

SEQ ID NO: 15 - DNA, 698 bp, Cryptococcus flavescens; identical to SEQ ID NO: 12

SEQ ID NO: 16 - DNA, 698 bp, Cryptococcus flavescens
tacccgatctcttcgatcatccgtcgctcggccaagcgcccttcgagctgccaatgctgctgtacgtctcgtttgtctcgcccgatcgccccaactcatggcgctaacattttgcagcgatgcacccccctcatcagcaagcgattcaacagtggcaaggtctctggacccgtcatcgggtacgcctgccccccgtctgctatacaagattcgacgagctcacagtcagtattgacttgggaacgactaactcctgtgtctcgtgagctatttctttgcggctgctgtcttgcggtcgcttaacatcagcatcttcgagaacggcggtgccaaggtccttgagaacgcggagggtgcccgaaccaccccatcggttgttgcctttaccaagggtgcgcatcgcgatttacgaagtggcaacacgctgacgtacagatggcgagcgattagtcggtcaaccggctcgacgacaggccgttgtcaacggtgaacacaccatctttgcctccaagcggtaagccggcaaatccgcctagaatgagccgcagaactgatcccctagactgatcggacgaaagttcaccgacgccgaggtccagaaggacatcaagaacgtcccctacaagattgtcgcccactctaacggtgacgcttgggtcgaggctcgaggacagaagtactcgccatcgcaaattggt

SEQ ID NO: 17 - DNA, 533 bp, Cryptococcus flavescens
ggaacgatggactcgatccggggtgggcccctcggctcgctcttccgacctgacaacttgtgagcttgggttcgaatagcgtccagagggtaccagaggctgacggtagcgtgtttggacagagcggagcgggaaacaactgggcgaagggtcactacaccgagggtgcgtacgcaaagccattatgctccacctgggctgtttcctgctcgcccactgaccccaggtgccgagctggtcgactcggtcctcgacgtcgtccgacgagaggcggagaacacggactgcctccagggtttccagatcacccactcgctcggtggaggaaccggagccggtatgggaacattgttgatctccaagatcagggaggagtacccggaccgaatgatgtgcaccatctcggtcgtaccctcgcccaaggtgcgtccaaggctcccgtggtgttgtacgatctaactcgcaggtgtccgacaccgttgtcgagccgtacaacgcgacgctctcggtccaccagcttgtggagaactctg

SEQ ID NO: 18 - DNA, 533 bp, Cryptococcus flavescens
ggaacgatggactcgatccggggtgggcccctcggctcgctcttccgacctgacaacttgtgagcttgggttcgaatagcgtccagagggtaccagaggctgacggtagcgtgtttggacagagcggagcgggaaacaactgggcgaagggtcactacaccgagggtgcgtacgcaaagccattatgctccacctgggctgtttcctgctcgcccactgaccccaggtgccgagctggtcgactcggtcctcgacgtcgtccgacgagaggcggagaacacggactgcctccagggtttccagatcacccactcgctcggtggaggaaccggagccggtatgggaacattgttgatctccaagatcagggaggagtacccggaccgaatgatgtgcaccttctcggtcgtcccctcgcccaaggtgcgtccaaggctcccgtggtgttgtacgatctaactcgcaggtgtccgacaccgttgtcgagccgtacaacgcgacgctctcggtccaccagcttgtggagaactctg

SEQ ID NO: 19 - DNA, 533 bp, Cryptococcus flavescens; identical to SEQ ID NO: 18

SEQ ID NO: 20 - DNA, 533 bp, Cryptococcus flavescens
ggaacgatggactcgatccggggtgggcccctcggctcgctcttccgacctgacaacttgtgagcttgggttcgaatagcgtccagagggtaccagaggctgacggtagcgtgtttggacagagcggagcgggaaacaactgggcgaagggtcactacaccgagggtgcgtacgcaaagccattatgctccacctgggctgtttcctgctcgcccactgaccccaggtgccgagctggtcgactcggtcctcgacgtcgtccgacgagaggcggagaacacggactgcctccagggtttccagatcacccactcgctcggtggaggaaccggagccggtatgggaacattgttgatctccaagatcagggaggagtacccggaccgaatgatgtgcaccttctcggtcgtaccctcgcccaaggtgcgtccaaggctcccgtggtgttgtacgatctaactcgcaggtgtccgacaccgttgtcgagccgtacaacgcgacgctctcggtccaccagcttgtggagaactctg

SEQ ID NO: 21 - DNA, 533 bp, Cryptococcus flavescens; identical to SEQ ID NO: 20

SEQ ID NO: 22 - DNA, 533 bp, Cryptococcus flavescens; identical to SEQ ID NO: 18

SEQ ID NO: 23 - DNA, 533 bp, Cryptococcus flavescens; identical to SEQ ID NO: 18

SEQ ID NO: 24 - DNA, 332 bp, Cryptococcus flavescens
agggagaagatcacgttgtgctggatcgcgtgtctaatgggtgcggtggttgggtttgcgacgatggggctgcagagggcattgtgtccggaggggtcgacgagtatattccggcggctgggggatgatcagagtgagtcgaggctgtggctgtcgttcggttgtggtgtgggcggtgggcggaggcatgggtgaagctagcggaaagcagcagagttgacatgcatggacaagcggaggtggacatggcggtcagcacctggaaagttccttgtcggagagaccgatctcgagcgcgcagcggaagaggcagatgagcgcgcgaagcctga

SEQ ID NO: 25 - DNA, 331 bp, Cryptococcus flavescens
agggagaagatcacgttgtgctggatcgcgtgtctaatgggtgcggtggttgggtttgcgacgatggggctgcagagggcattgtgtccggaggggtcgacgagtatattccggcggctgggggatgatcagagtgagtcgaggctgtggctgtcgttcggttgtggtgtgggcggtgggcggaggcatgggtgaagctagcggaaagcagcagagttgacatgcttggactagcggaggtggacatggcggtcagcacctgaaagttccttgtcggagagaccgatctcgagcgcgcagcggaagaggcagatgagcgcgcgaagcctga

SEQ ID NO: 26 - DNA, 332 bp, Cryptococcus flavescens
agggagaagatcacgttgtgctggatcgcgtgtctgatgggtgcggtggttgggtttgcgacgatggggctgcagagggcactgtgtccggaggggtcgacgagtatattccggcggctgggggatgatcagagtgagtcgaggctgtggctgtcgttcggttgtggtgtgggcggtgggcggaggcatgggtgaagctagcggaaagcagcagagttgacatgcttggactagcggaggtggacatggcggtcagcacctggaaagttccttgtcggagagaccaatctcgagcgcgcagcggaagaggctgatgagcgcgcgaagcctga

SEQ ID NO: 27 - DNA, 332 bp, Cryptococcus flavescens
agggagaagatcacgttgtgctggatcgcgtgtctaatgggtgcggtggttgggtttgcgacgatggggctgcagagggcattgtgtccggaggggtcgacgagtatattccggcggctgggggatgatcagagtgagtcgaggctgtggctgtcgttcggttgtggtgtgggcggtgggcggaggcatgggtgaagctagcggaaagcagcagagttgacatgcttggactagcggaggtggacatggcggtcagcacctggaaagttccttgtcggagagaccgatctcgagcgcgcagcggaagaggcagatgagcgcgcgaagcctga

SEQ ID NO: 28 - DNA, 332 bp, Cryptococcus flavescens
agggagaagatcacgttgtgctggatcgcgtgtctaatgggtgcggtggttgggtttgcgacgatggggctgcagagggcattgtgtccggaggggtcgacgagtatattccggcggctgggggatgatcagagtgagtcgaggctgtggctgtcgttcggttgtggtgtgggcggtgggcggaggcatgggtgaagctagcggaaagcagcagagttgacatgcatggactagcggaggtggacatggcggtcagcacctggaaagttccttgtcggagagaccgatctcgagcgcgcagcggaagaggcagatgagcgcgcgaagcctga

SEQ ID NO: 29 - DNA, 332 bp, Cryptococcus flavescens; identical to SEQ ID NO: 27

SEQ ID NO: 30 - DNA, 332 bp, Cryptococcus flavescens; identical to SEQ ID NO: 26

SEQ ID NO: 31 - DNA, 591 bp, Cryptococcus flavescens
caagaccctccttgacgccatcgacgccattgagcccccgacccgacccaccgacaagccgctccgacttcctctccaggatgtctacaagatcggtggtatcggcacagtccccgtcggccgagtcgagaccggtgtgatcaagggagggtgagtgatcgtcttttgccacccgccacccgctacccgtcaccttcgcgactaccttgacccatcgtgtacgcacttgcagacgctgatatcagaatggttgttaccttcgccccgaccaacgtcaccaccgaggtcaagtccgtcgagatgcaccacgagcaggttagtcccgccacccgccacttttttgccactcaccccttccaccccttccctctctttccttcgctgacatccagatccccgagggtctcccgggagacaacgtcggcttcaacgtcaagaacgtctccatcaaggacatccgacgaggaaacgtctgctccgactcgaagaacgaccccgccaaggaggccgcttccttcaacgcccaggtcattgtcctcaaccacccgggacagatcggtgccggttacaccccggtccttgactgccaca

SEQ ID NO: 32 - DNA, 591 bp, Cryptococcus flavescens; identical to SEQ ID NO: 31

SEQ ID NO: 33 - DNA, 590 bp, Cryptococcus flavescens
caagacctccttgacgccatcgacgccattgagcccccgacccgacccaccgacaagccgctccgacttcctctccaggatgtctacaagatcggtggtatcggcacagtccccgtcggccgagtcgagaccggtgtgatcaagggagggtaagtgatcgtcttttgccacccgccacccgctacccgtcaccttcgcgacaaccttgacccatcgtgtacgcacttgcagacgctgatatcagaatggttgttaccttcgccccgaccaacgtcaccaccgaggtcaagtccgtcgagatgcaccacgagcaggttagtcccgccacccgccacttttttgccactcaccccttccaccccttccctctctttccttcgctgacatccagatccccgagggtctcccgggagacaacgtcggcttcaacgtcaagaacgtctccatcaaggacatccgacgaggaaacgtctgctccgactcgaagaacgaccccgccaaggaggccgcttccttcaacgcccaggtcattgtcctcaaccacccgggacagatcggtgccggttacaccccggtccttgactgccaca

SEQ ID NO: 34 - DNA, 591 bp, Cryptococcus flavescens; identical to SEQ ID NO: 31

SEQ ID NO: 35 - DNA, 591 bp, Cryptococcus flavescens; identical to SEQ ID NO: 31

SEQ ID NO: 36 - DNA, 591 bp, Cryptococcus flavescens; identical to SEQ ID NO: 31

SEQ ID NO: 37 - DNA, 590 bp, Cryptococcus flavescens; identical to SEQ ID NO: 33

SEQ ID NO: 38 - DNA, 697 bp, Cryptococcus flavescens
tacccgatctcttcgatcatccgtcgcccggccaagcgccctccgagctgccaacgctgctgtacgtctcgtttgtctcgcccgatcgctccaactcatggcgctaacaattcgcagcgatgcacccccctcatcagcaagcgattcaacagtggcaaggtctctgggcccgtcatcgggtacgcccgccccccgtctgctatacaagagccgacgagctaacagtcagtattgacttgggaacgaccaactcctgtgtctcgtgagctatctctttgctgctgctgtcgtgcggtcgctaaacatcagcattttcgagaacggcggtgccaaggtccttgagaacgcggagggtgcacgaaccaccccatcggttgttgccttcaccaagggtgcgcatatcgatctacgaagctgcaacacgctgacgtctagatggcgagcgattagtcggtcaaccggctcgacgacaggccgttgtcaacggtgaacacaccatctttgcctccaagcggtaagccggcaaacactcctcaaccggcagcagagctgatccctaagactgatcggacgaaagttcaccgacgccgaggtccagaaggacatcaagaacgtcccgtacaagattgtcgcccactctaacggtgacgcttgggtcgaggctcgaggacagaagtactcgccatcgcaaattggt

SEQ ID NO: 39 - DNA, 697 bp, Cryptococcus flavescens
tacccgatctcttcgatcatccgtcgcccggccaagcgccctccgagctgccaacgctgctgtacgtctcgtttgtctcgcccgatcgctccaactcatggcgctaacaatttgcagcgatgcacccccctcatcagcaagcgattcaacagtggcaaggtctctgggcccgtcatcgggtacgcccgccccccgtctgctatacaagagccgacgagctaacagtcagtattgacttgggaacgaccaactcctgtgtctcgtgagctatctctttgctgctgctgtcgtgcggtcgctaaacatcagcattttcgagaacggcggtgccaaggtccttgagaacgcggagggtgcacgaaccaccccatcggttgttgccttcaccaagggtgcgcataccgatctacgaagctgcaacacgctgacgtacagatggcgagcgattagtcggtcaaccggctcgacgacaggccgttgtcaacggtgaacacaccatctttgcctccaagcggtgagccggcaaacactcctcaaccggcggcagagctgatccctaagactgatcggacgaaagttcaccgacgccgaggtccagaaggacatcaagaacgtcccgtacaagattgtcgcccactctaacggtgacgcttgggtcgaggctcgaggacagaagtactcgccatcgcaaattggt

SEQ ID NO: 40 - DNA, 697 bp, Cryptococcus flavescens; identical to SEQ ID NO: 39

SEQ ID NO: 41 - DNA, 697 bp, Cryptococcus flavescens; identical to SEQ ID NO: 38

SEQ ID NO: 42 - DNA, 697 bp, Cryptococcus flavescens; identical to SEQ ID NO: 38

SEQ ID NO: 43 - DNA, 697 bp, Cryptococcus flavescens; identical to SEQ ID NO: 38

SEQ ID NO: 44 - DNA, 697 bp, Cryptococcus flavescens; identical to SEQ ID NO: 39

SEQ ID NO: 45 - DNA, 22 bp, Artificial Sequence
  Description of Artificial Sequence Synthetic primer
tgcttccacgctgtgatgccac

SEQ ID NO: 46 - DNA, 22 bp, Artificial Sequence
  Description of Artificial Sequence Synthetic primer
tgtacgaaccgtcggcctggat

SEQ ID NO: 47 - DNA, 22 bp, Artificial Sequence
  Description of Artificial Sequence Synthetic primer
tgcgtgcgcggttccatcatac

SEQ ID NO: 48 - DNA, 22 bp, Artificial Sequence
  Description of Artificial Sequence Synthetic primer
aaaccctggaggcagtccgtgt

SEQ ID NO: 49 - DNA, 22 bp, Artificial Sequence
  Description of Artificial Sequence Synthetic primer
tcaacacgcttccgccctcaac

SEQ ID NO: 50 - DNA, 22 bp, Artificial Sequence
  Description of Artificial Sequence Synthetic primer
gcctgaagcccggatttgcgat

SEQ ID NO: 51 - DNA, 22 bp, Artificial Sequence
  Description of Artificial Sequence Synthetic primer
gcgtggcgggagaaattgacca

SEQ ID NO: 52 - DNA, 22 bp, Artificial Sequence
  Description of Artificial Sequence Synthetic primer
gttgtgactggcaagctccgca

SEQ ID NO: 53 - DNA, 22 bp, Artificial Sequence
  Description of Artificial Sequence Synthetic primer
tgacgccgccattgtcaagctc

SEQ ID NO: 54 - DNA, 22 bp, Artificial Sequence
  Description of Artificial Sequence Synthetic primer
ggcaacggtctgtcgcatgtct

SEQ ID NO: 55 - DNA, 22 bp, Artificial Sequence
  Description of Artificial Sequence Synthetic primer
tcgtgtacgtccttgcagacgc

SEQ ID NO: 56 - DNA, 22 bp, Artificial Sequence
  Description of Artificial Sequence Synthetic primer
aaggcgggactaacctgctcgt

SEQ ID NO: 57 - DNA, 22 bp, Artificial Sequence
  Description of Artificial Sequence Synthetic primer
gacttcaacgacgcccagcgat

SEQ ID NO: 58 - DNA, 22 bp, Artificial Sequence
  Description of Artificial Sequence Synthetic primer
acgatgatctgcgactcggcct

SEQ ID NO: 59 - DNA, 22 bp, Artificial Sequence
  Description of Artificial Sequence Synthetic primer
agagtccatccgcgcagtgcta

SEQ ID NO: 60 - DNA, 22 bp, Artificial Sequence
  Description of Artificial Sequence Synthetic primer
tcttgccactcacaggccggaa

SEQ ID NO: 61 - DNA, 22 bp, Artificial Sequence
  Description of Artificial Sequence Synthetic primer
agggcgagcgactgattggtga

SEQ ID NO: 62 - DNA, 22 bp, Artificial Sequence
  Description of Artificial Sequence Synthetic primer
tcgaagacggtgttggtcgggt

SEQ ID NO: 63 - DNA, 22 bp, Artificial Sequence
  Description of Artificial Sequence Synthetic primer
accaaggacgctggtgcgattg

SEQ ID NO: 64 - DNA, 22 bp, Artificial Sequence
  Description of Artificial Sequence Synthetic primer
cgtttcgctcctcgctcgtctt

SEQ ID NO: 65 - DNA, 22 bp, Artificial Sequence
  Description of Artificial Sequence Synthetic primer
cggcttaccgcttggaggcaaa

SEQ ID NO: 66 - DNA, 22 bp, Artificial Sequence
  Description of Artificial Sequence Synthetic primer
acgaagtggcaacacgctgacg

SEQ ID NO: 67 - DNA, 22 bp, Artificial Sequence
  Description of Artificial Sequence Synthetic primer
cgtcagcgtgttgccacttcgt

SEQ ID NO: 68 - DNA, 22 bp, Artificial Sequence
  Description of Artificial Sequence Synthetic primer
gctgctgtcttgcggtcgctta

SEQ ID NO: 69 - DNA, 22 bp, Artificial Sequence
  Description of Artificial Sequence Synthetic primer
tctttgaacgcaccttgcgcct

SEQ ID NO: 70 - DNA, 22 bp, Artificial Sequence
  Description of Artificial Sequence Synthetic primer
tgctggcagacacccaagtcca

SEQ ID NO: 71 - DNA, 22 bp, Artificial Sequence
  Description of Artificial Sequence Synthetic primer
aggcttggacttgggtgtctgc

SEQ ID NO: 72 - DNA, 21 bp, Artificial Sequence
  Description of Artificial Sequence Synthetic primer
aaggtgcgcgatggcaggttt

SEQ ID NO: 73 - DNA, 20 bp, Artificial Sequence
  Description of Artificial Sequence Synthetic primer
tcctccgcttattgatatgc

SEQ ID NO: 74 - DNA, 22 bp, Artificial Sequence
  Description of Artificial Sequence Synthetic primer
ggaagtaaaagtcgtaacaagg

SEQ ID NO: 75 - DNA, 25 bp, Artificial Sequence
  Description of Artificial Sequence Synthetic primer
gggcggtcttgattgacttggaacc

SEQ ID NO: 76 - DNA, 25 bp, Artificial Sequence
  Description of Artificial Sequence Synthetic primer
tcgttgtcgatacagaaggtcgcgt

SEQ ID NO: 77 - DNA, 25 bp, Artificial Sequence
  Description of Artificial Sequence Synthetic primer
acaggcttggagggagaagatcacg

SEQ ID NO: 78 - DNA, 25 bp, Artificial Sequence
  Description of Artificial Sequence Synthetic primer
cgaacgaggtagaatcaggcttcgc

SEQ ID NO: 79 - DNA, 25 bp, Artificial Sequence
  Description of Artificial Sequence Synthetic primer
cgtggtacaagggatggaccaagga

SEQ ID NO: 80 - DNA, 25 bp, Artificial Sequence
  Description of Artificial Sequence Synthetic primer
caatgtgggcagtgtggcagtcaag

SEQ ID NO: 81 - DNA, 25 bp, Artificial Sequence
  Description of Artificial Sequence Synthetic primer
atgtacgccgctacccgatctcttc

SEQ ID NO: 82 - DNA, 25 bp, Artificial Sequence
  Description of Artificial Sequence Synthetic primer
cgacaaaggcaccaatttgcgatgg

SEQ ID NO: 83 - DNA, 25 bp, Artificial Sequence
  Description of Artificial Sequence Synthetic primer
aagaagggagtgaggaagcagctcg

SEQ ID NO: 84 - DNA, 25 bp, Artificial Sequence
  Description of Artificial Sequence Synthetic primer
acttgttcgcctcttggtagacgct

SEQ ID NO: 85 - DNA, 807 bp, Cryptococcus flavescens
gaagcagctcgaggaggaatacggggtcagcttgtcggcgagaaaggaaagcatcaacaggatcatcgagcgggttttggctggtgagtggctttggcaaggatggaggtgatagcgctgatgagcagagtgatgacgatgatgtgaatgacgacgacgacgacgacgatgaagacggacactgatacgattatggacaatctgcttccattgacagacgacactgcgacgtgttacggacggagcttttagcatgacgcttacgcatgaccactgagctatgcatgatggatatgtttcgcggctgctctggcccactgacggacgggcactagccagactggggtcctttcggacatggtcataacgatgaccggagtcgccgcggtctgcctgacgacgggcgggaggggttgctcgacgacatgccgtgtacgccgagtgcttcgagtctagctgtgatgcacgggggcacgcgggagtctgaccgttgtgatgcatgcacaaagctctgtgcgcggtgtacggtgtgcttacatgctttttaccctctctgccgcaccttcatatcgtctgcgacggcccacctactgtggccagatctttggcattggatccctactgatcaatcaacactctccaccgacacaaccgcatcgctgatagcgtaatggtgcacacagacgatcttcccaacacccctgcgaaagtgttgactagctcccgcgcacgcaacgtcagccgagcgacgccacgagtcgcgcccaagatagagtatcgagaccccgacacgccatcgccgtcgcc

SEQ ID NO: 86 - DNA, 807 bp, Cryptococcus flavescens; identical to SEQ ID NO: 85

SEQ ID NO: 87 - DNA, 807 bp, Cryptococcus flavescens; identical to SEQ ID NO: 85

SEQ ID NO: 88 - DNA, 807 bp, Cryptococcus flavescens; identical to SEQ ID NO: 85

SEQ ID NO: 89 - DNA, 810 bp, Cryptococcus flavescens
gaagcagctcgaggaggaatacggggtcagcttgtcggcgagaaaggaaagcatcaacaggatcatcgagcgggttttggctggtgagtggctttggcaaggatggaggtgatagcgctgatgagcagagtgatgacgatgatgtgaatgacgacgacgacgacgacgacgatgaagacggacactgatacgattatggacaatctgcttccattgacagacgacactgcgacgtgttacggacggagcttttagcatgacgcttacgcatgaccactgagctatgcatgatggatatgtttcgcggctgctctggcccactgacggacgggcactagccagactggggtcctttcggacatggtcataacgatgaccggagtcgccgcggtctgcctgacgacgggcgggaggggttgctcgacgacatgccgtgtacgccgagtgcttcgagtctagctgtgatgcacgggggcacgcgggagtctgaccgttgtgatgcatgcacaaagctctgtgcgcggtgtacggtgtgcttacatgctctttaccctctctgccgcaccttcatatcgtctgcgacggcccacctactgtggccagatctttggcattggatccctactgatcaatcaacactctccaccgacacaaacgcatcgctgatagcgtaatggtgcacacagacgatcttcccaacacccctgcgaaagtgttgactagctcccgcgcacgcaatgtcagccgagcgacgccacgagtcgcgcccaagatagagtatcgagaccccgacacgccatcgccgtcgcc

SEQ ID NO: 90 - DNA, 761 bp, Cryptococcus flavescens
gaagcagctcgaggaggagtacggggtcagcttgtcggcgaggaaggagagcatcaacagaatcatagagcgggttttggctggtgagtggcttctgtgccgatgacccgtgccgacgctgatggacagagtgatgatgatgatgatgtgaatgacgacgacgaaaacggacattgatacgattacggacaatctgcctcattgacagacgacactgcgacgtgttacggatggagcttttagcatagcgctcacgcttgaccactgagctatgcatgaaagatgtgtttcgcggctggggtcctttcggccatgttcacaacgatgaccggggtcgacgcgggtctgcttgacgacgggcgggagagattgctgaacgacatgcccgtacgccgagtgcttacatcctggctttgatgcacgggggacgcaggagtttggcagtcgcgacgcatgctcaaagctctgtgcgcgtcgcgcggggtgtctacatgctctttaacctctctaccgcaccctcttctcatcttcgatcgcccacctactaaagccagatttttggcatcgagtccccactgtccaatcgagaccttccaccgacactaccgcatcgctgacagcgttatggtgcacacagacgattctcccagcacccctccgaaagcgtcgactgcctcccgcgcacgcaacgtcagccgagcaacgccacgagtcacgcccaaattggagtatcgagaccccgatacgccatcgccgtcacc

SEQ ID NO: 91 - DNA, 50 bp, Artificial Sequence
  Description of Artificial Sequence Synthetic consensus oligonucleotide
cgtcagcgtgttgcmrcttcgtaratcgskatgcgcacccttggtraagg

SEQ ID NO: 92 - DNA, 50 bp, Cryptococcus flavescens
cgtcagcgtgttgccacttcgtaaatcgcgatgcgcacccttggtaaagg

SEQ ID NO: 93 - DNA, 50 bp, Cryptococcus flavescens
cgtcagcgtgttgcagcttcgtagatcgatatgcgcacccttggtgaagg

SEQ ID NO: 94 - DNA, 50 bp, Cryptococcus flavescens
cgtcagcgtgttgcagcttcgtagatcggtatgcgcacccttggtgaagg

SEQ ID NO: 95 - DNA, 50 bp, Artificial Sequence
  Description of Artificial Sequence Synthetic consensus oligonucleotide
caacaaccgatggggtggttcgkgcaccctccgcgttctcaaggaccttg

SEQ ID NO: 96 - DNA, 50 bp, Cryptococcus flavescens
caacaaccgatggggtggttcgggcaccctccgcgttctcaaggaccttg

SEQ ID NO: 97 - DNA, 50 bp, Cryptococcus flavescens
caacaaccgatggggtggttcgtgcaccctccgcgttctcaaggaccttg

SEQ ID NO: 98 - DNA, 50 bp, Artificial Sequence
  Description of Artificial Sequence Synthetic consensus oligonucleotide
gcaccgccgttctcgaaratgctgatgttwagcgaccgcamgacagcagc

SEQ ID NO: 99 - DNA, 50 bp, Cryptococcus flavescens
gcaccgccgttctcgaagatgctgatgttaagcgaccgcaagacagcagc

SEQ ID NO: 100 - DNA, 50 bp, Cryptococcus flavescens
gcaccgccgttctcgaaaatgctgatgtttagcgaccgcacgacagcagc